(19) United States
(12) Patent Application Publication
(10) Pub. No.: US 2025/0154245 A1
(43) Pub. Date: May 15, 2025

(54) MONOCLONAL ANTI-IL-1RACP ANTIBODIES

(71) Applicant: SANOFI BIOTECHNOLOGY, Gentilly (FR)

(72) Inventors: Stephan Fischer, Weilheim (DE); Michael Brandt, München (DE)

(21) Appl. No.: 19/025,463

(22) Filed: Jan. 16, 2025

Related U.S. Application Data
(62) Division of application No. 17/522,600, filed on Nov. 9, 2021, now Pat. No. 12,240,898.
Application No. 17/522,600 is a continuation of application No. 16/898,074, filed on Jun. 10, 2020, now Pat. No. 11,198,728.
Application No. 16/898,074 is a continuation of application No. 15/739,410, filed on Dec. 22, 2017, now Pat. No. 10,906,971.

(30) Foreign Application Priority Data
Jun. 26, 2015 (EP) 15174184.0
Dec. 17, 2015 (EP) 15200772.0

(51) Int. Cl. C07K 16/24; A61K 39/00; C07K 16/28; C12N 5/12

(52) U.S. Cl. CPC C07K 16/245; C07K 16/2866; C12N 5/12; A61K 2039/505; C07K 2317/20; C07K 2317/33; C07K 2317/565; C07K 2317/732; C07K 2317/76

(57) ABSTRACT

Monoclonal antibody that specifically binds IL-1RAcP, or an antigen binding fragment thereof, comprising:
a) a heavy chain variable region (VH) comprising CDR1H, CDR2H and/or CDR3H, wherein the CDR1H region comprises an amino acid sequence selected from the group of SEQ ID NO: 155-231,
wherein the CDR2H region comprises an amino acid sequence selected from the group of SEQ ID NO: 232-308,
and wherein the CDR3H region comprises an amino acid sequence selected from the group of SEQ ID NO: 309-385; and
b) a light chain variable region (VL) comprising CDR1L, CDR2L and/or CDR3L, wherein the CDR1L region comprises an amino acid sequence selected from the group of SEQ ID NO: 386-462,
wherein the CDRL2 region comprises an amino acid sequence selected from the group of SEQ ID NO: 463-539,
and wherein the CDR3L region comprises an amino acid sequence selected from the group of SEQ ID NO: 540-616

The monoclonal antibody is characterized in that it inhibits IL-1RAcP induced NFkB activity, useful in treatment of IL-1RAcP related diseases.

### RELATED APPLICATIONS

This application is a divisional of U.S. patent application Ser. No. 17/522,600, filed Nov. 9, 2021, which is a continuation of U.S. patent application Ser. No. 16/898,074, filed Jun. 10, 2020, now U.S. Pat. No. 11,198,728, which is a continuation of U.S. patent application Ser. No. 15/739,410, filed Dec. 22, 2017, now U.S. Pat. No. 10,906,971, which is a 35 U.S.C. § 371 filing of International Patent Application No. PCT/EP2016/064588, filed Jun. 23, 2016, which claims priority to European Patent Application Nos. 15200772.0, filed Dec. 17, 2015, and 15174184.0, filed Jun. 26, 2015, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Jan. 13, 2025, is named 760592_SA9-292USCON2DIV_ST26.xml and is 552,620 bytes in size.

### FIELD OF THE INVENTION

The present invention relates to monoclonal anti-IL-1RAcP antibodies, methods for the production and uses thereof.

### BACKGROUND

Human IL-1RAcP (Q9NPH3 (IL1AP_HUMAN, UniProtKB/Swiss-Prot) is an accessory protein that is required to transmit signals through receptors of the IL-1 family. The interleukin-1 receptor complex is a heterodimer of IL-1R1 and IL-1RAcP. Upon binding of IL-1, IL-1R1 associates with IL-1RAcP forming a functional signaling receptor complex, which stimulates NFkB activity.

IL-33, its receptor ST2, and IL-1RAcP form also a complex (IL-33/ST2/IL-1RAcP) with a similar activity in regard to NFkB activation as the IL-1β/IL-1R1/IL-1RAcP complex. IL-36 (IL-36α (IL-1F6), IL-36α (IL-1F8), and IL-36γ (IL-1F9)), their receptor IL-36R, and IL-1RAcP form also a complex (IL-36/Il-36R/IL-1RAcP) with a similar activity in regard to NFkB activation as the IL-1β/IL-1R1/IL-1RAcP complex.

WO199623067 relates to an IL-1RAcP antibody, which binds specifically to murine IL-1 receptor accessory protein. Examples 15 and 16 describe the attempt to generate anti-human IL-1RAcP antibodies, which neutralize IL-1 biological activity. However, no such antibody is provided by WO199623067 and example 16, describing an IL-1 induced IL-6 assay is only hypothetical. Do-Young Yoon D-Y and Charles A. Dinarello CA describe in J. Immunol. 1998; 160:3170-3179 polyclonal antibodies to domains II and III of the murine IL-1RAcP which inhibit IL-1beta activity but not binding. However, at higher concentrations of IL-1beta (1000 pg/ml), this polyclonal antiserum did not block the proliferation of D10S cells. (D10S is a subclone of the murine D10.G4.1 helper T-cell which proliferates to subfemtomolar (attomolar) concentrations of IL-1 beta or alpha in the absence of mitogens, cf. Orencole S F and Dinarello C A; Cytokine 1 (1989) 14-22). Jaras M. et al., PNAS 107 (2010) 16280-16285 describe the use of rabbit polyclonal anti-IL1RAcP antibody KMT-1for killing CML stems cell. This antibody induces ADCC in an IL1RAcP-independent manner caused by its rabbit Fc part. Jaras et al. expect that “potential future therapeutic IL1RAP-targeting antibodies are expected to show low toxicity on normal hematopoietic cells”. Polyclonal rabbit antibodies against murine IL-1RAcP were also mentioned in Do-Young Yoon and Charles A. Dinarello, Journal of Biochemistry and Molecular Biology, Vol. 40, No. 4, July 2007, pp. 562-570.

A rabbit polyclonal antibody binding to mouse, rat, and human IL1RAcP (ab8110) is commercially available from Abcam, Cambridge, Massachusetts, USA. Abcam's ab8109 binds only to human IL1RAcP. BALAGURUNATHAN Y. et al., Mol. Cancer Ther. 7 (2008) 3071-3080 mentions the use of Abcam's polyclonal rabbit anti-IL1 RAP antibody for identifying pancreatic tumor cells.

WO2002064630 relates also to IL-1RAcP and its use, but no antibodies against IL-1RAcP are described. WO2004022718 and WO2009120903 mention theoretically that antibodies against CSF1R, IL13RA1, IL1RAP, IFNAR1, IL5R, INSR, IL1RL1, LTK, and TACSTD1 could be generated according to the state of the art. However, here also no antibody against IL-1RAcP is described. WO2011021014 and WO 2012098407 (US20140017167) relate to the polyclonal rabbit anti-human IL-1RAcP antiserum KMT-1 (see Jaras et al. 2010) and its use. WO2014100772 relates to an anti-IL-1RAcP antibody binding to IL-1RAcP. However, no activity in regard to inhibition of any functional signaling receptor complex (like IL-1β/IL-1R1/IL-1RAcP) which stimulates NFkB activity is described. U.S. Pat. No. 6,280,955 relates to IL-1RAcP and its use, but again no antibodies against IL-1RAcP are described. U.S. Pat. No. 7,390,880 mentions a N-terminal fragment of IL1RAcP, but describe also no antibodies against IL-1RAcP.

WO2004100987 relates to the use of an interleukin-I (IL-1) antagonist in the preparation of a medicament for the treatment of neointimal hyperplasia and to the use of an IL-1 antagonist for the treatment of neointimal hyperplasia. As such an antagonist an anti-IL-1RAcP antibody is suggested but not further described. US2003026806 relates to antibodies binding to IL-1. WO2002064630 relates also to an IL-1 antagonist ant to IL-1RAcP protein. Though to the use of IL-1RAcP for screening for IL-1RAcP antagonists are mentioned, no such method or antagonist is disclosed.

WO2003014309 relates to the use of IL-1RAcP protein to treat chronic myelogenous leukemia. WO2013023015 relates to a method for determining the prognosis of AML and to a method for treating AML by administering an agent inhibiting expression or activity of IL-1RAcP in early stem cells. As such an agent shRNA of IL-1RAcP is mentioned.

Human NF-kB is an important regulator of expression of several genes involved in inflammation, immune response and apoptosis and therefore dysfunction of NFkB is involved in the in the pathology of various diseases, including autoimmune diseases, neurodegenerative diseases, inflammation, and cancers. For example, NF-kB pathway is an important target in the treatment of OA and inhibition of human IL1beta stimulated human NFkB activity may be for example important in the treatment of osteoarthritis. Therefore, a monoclonal antibody which regulates the human NFkB pathway via inhibiting the signaling activity of the human IL-1R1/IL-1RAcP complex would be a valuable therapeutic agent in treating various diseases of human beings.

However, attempts since about more than 15 years to generate functional monoclonal antibodies against human IL1RAcP failed and such need exists therefore still today.

### SUMMARY OF THE INVENTION

The invention provides a monoclonal antibody against human IL-1RAcP. Preferably the antibody according to the invention binds in addition to murine IL-1RAcP.

The invention provides a monoclonal antibody against human IL-1RAcP characterized in inhibiting IL-1RAcP induced NFkB activity.

The invention provides a monoclonal antibody specifically binding to human IL-1RAcP. Preferably the antibody according to the invention binds in addition to murine IL-1RAcP.

The invention provides a monoclonal antibody specifically binding to human IL-1RAcP characterized in inhibiting IL-1RAcP induced NFkB activity. Preferably, the antibody according to the invention inhibits in addition murine IL-1RAcP induced murine NFkB activity.

The invention provides a monoclonal antibody against human IL-1RAcP characterized in inhibiting NFkB activity stimulated by IL-1alpha, IL-1beta, IL-33 and/or IL-36. The invention provides a monoclonal antibody against human IL-1RAcP characterized in inhibiting IL1alpha stimulated NFkB activity. The invention provides a monoclonal antibody against human IL-1RAcP characterized in inhibiting IL1beta stimulated NFkB activity.

The invention provides a monoclonal antibody against human IL-1RAcP characterized in inhibiting IL33 stimulated NFkB activity. The invention provides a monoclonal antibody against human IL-1RAcP characterized in inhibiting IL36 stimulated NFkB activity.

The invention provides a monoclonal antibody against human IL-1RAcP characterized in inhibiting NFkB activity stimulated by a complex selected from the group consisting of IL-1β/IL-1R1/IL-1RAcP, IL-1α/IL-1R1/IL-1RAcP IL-33/ST2/IL-1RAcP, and IL-36/Il-36R/IL-1RAcP.

Preferably, the antibody according to the invention is characterized in binding to murine IL-1RAcP and inhibiting murine IL-1RAcP induced murine NFkB activity.

Preferably, the antibody according to the invention is characterized in inhibiting in a concentration of 5 μg/ml (rabbit IgG isotype has a molecular weight of 150 KD) NFkB activity in 293T/17 cell lysates (293T/17 [HEK 293T/17] (ATCC® CRL-11268™)) stimulated with 0.5 μg/ml human IL-1alpha, IL-1beta, IL-33 and/or IL-36 (molecular weight see UniProtKB/Swiss-Prot), for 70% or more, preferably for 80% or more, preferably for 90% and more, and more preferably for 95% or more, related to the same assay without said antibody according to the invention.

Preferably the antibody according to the invention is characterized in inhibiting in a concentration of 5 μg/ml NFkB activity in respective mouse cell line lysates stimulated with 0.5 μg/ml murine IL-1alpha, IL-1beta, IL-33 and/or IL-36 (molecular weight see UniProtKB/Swiss-Prot), for 70% or more, preferably for 80% or more, preferably for 90% and more, and more preferably for 95% or more, related to the same assay without said antibody according to the invention.

Preferably the antibody according to the invention is characterized in exhibiting an ADCC reduced to at least 20% or lower, preferably to at least 10% or lower, of the ADCC induced by the antibody according to the invention comprising a wild-type human IgG Fc region.

Preferably the antibody according to the invention is characterized in exhibiting a reduced affinity to the human FcγRIIIA and/or FcγRIIA and/or FcγRI compared to an antibody according to the invention comprising the wildtype IgG Fc region, and wherein the ADCC induced by said antibody according to the invention is reduced to at least 20% of the ADCC induced by the antibody according to the invention comprising a wild-type human IgG Fc region.

Preferably the antibody according to the invention has a decreased effector function, like decreased ADCC and/or C1q binding. In particular the invention provides an antibody according to the invention comprising an Fc variant of a wild-type human IgG Fc region, said Fc variant comprising an amino acid substitution at position Pro329 and at least one further amino acid substitution, wherein the residues are numbered according to the EU index of Kabat, and wherein said antibody according to the invention exhibits a reduced affinity to the human FcγRIIIA and/or FcγRIIA and/or FcγRI compared to an antibody according to the invention comprising the wildtype IgG Fc region, and wherein the ADCC induced by said antibody according to the invention is reduced to at least 20% of the ADCC induced by the antibody according to the invention comprising a wild-type human IgG Fc region.

In a specific embodiment Pro329 of a wild-type human Fc region in the polypeptide described above is substituted with glycine or arginine or an amino acid residue large enough to destroy the proline sandwich within the Fcγ receptor interface, that is formed between the proline329 of the Fc and tryptophane residues Trp 87 and Tip 110 of FcγRIII (Sondermann et al.: Nature 406, 267-273 (20 Jul. 2000)). In a further aspect of the invention the at least one further amino acid substitution in the Fc variant is selected from the group consisting of S228P, E233P, L234A, L235A, L235E, N297A, N297D, or P331S and still in another embodiment said at least one further amino acid substitution is L234A and L235A of the human IgG1 Fc region or S228P and L235E of the human IgG4 Fc region.

In another aspect of the invention the antibody according to the invention provided exhibits a reduced affinity to at least one further receptor of the group comprising the human receptors FcγI, FcγIIA and C1q compared to the antibody according to the invention comprising a wild-type human IgG Fc region. In still another aspect of the invention the antibody according to the invention comprises a human IgG1 or IgG4 Fc region.

A further aspect of the invention is a use of an antibody according to the invention comprising an Fc variant of a wild-type human IgG Fc region, said antibody according to the invention having Pro329 of the human IgG Fc region substituted with glycine, wherein the residues are numbered according to the EU index of Kabat, wherein said antibody according to the invention exhibits a reduced affinity to the human FcγRIIIA and FcγRIIA for down-modulation of ADCC to at least 20% of the ADCC induced by the antibody according to the invention comprising the wildtype human IgG Fc region, and/or for down-modulation of ADCC.

Another aspect of the invention is use of an antibody according to the invention comprising an Fc variant of a wild-type human IgG Fc region, said antibody according to the invention having Pro329 of the human IgG Fc region substituted with glycine and wherein the Fc variant comprises at least two further amino acid substitutions at L234A and L235A of the human IgGI Fc region or S228P and L235E of the human IgG4 Fc region, wherein the residues are numbered according to the EU index of Kabat, wherein said antibody according to the invention exhibits a reduced affinity to the human FcγRIIIA and FcγRIIA, for down-modulation of ADCC to at least 20% of the ADCC induced by the antibody according to the invention comprising the wildtype human IgG Fc region, and/or for down-modulation of ADCC.

In another aspect of the invention a method of treating an individual having a disease is provided, wherein said individual is treated with an antibody according to the invention, said antibody according to the invention having Pro329 of the human IgG Fc region substituted with glycine, wherein the residues are numbered according to the EU index of Kabat, wherein said antibody according to the invention is characterized by a strongly reduced binding FcγRIIIA and/or FcγRIIA compared to an antibody according to the invention comprising a wildtype human IgG Fc region, comprising administering to the individual an effective amount of said antibody according to the invention.

In still another aspect of the invention the antibody according to the invention used in said method comprises at least two further amino acid substitutions at L234A and L235A of the human IgGI Fc region or S228P and L235E of the human IgG4 Fc region.

The invention provides preferably an antibody against human IL-1RAcP, characterized in that the heavy chain variable (VH) region is at least 90% identical to a VH region selected from the group consisting of VH regions of SEQ ID NO: 1 to 77.

The invention provides preferably an antibody against human IL-1RAcP, characterized in that the light chain variable (VL) region is at least 90% identical to a VL region selected from the group consisting of VL regions of SEQ ID NO: 78 to 154.

The invention provides preferably an antibody according to the invention, characterized in that its VH region is at least 90% identical to a VH region of SEQ ID NO: 1+n and its VL region is at least 90% identical to a VL region of SEQ ID NO: 78+n, wherein n is a number selected from the group consisting of 0 to 76.

The invention provides preferably an antibody according to the invention, characterized in that its VH region is selected from the group consisting of VH regions of SEQ ID NO: 1+n and its VL region is selected from the group consisting of VL regions of SEQ ID NO: 78+n, wherein n is a number selected from the group consisting of 0 to 76.

The invention provides preferably an antibody according to the invention, characterized in that the antibody comprises a VH region selected from the group of VH regions comprising a CDR1H region of SEQ ID NO: 155+n, a CDR2H region of SEQ ID NO: 232+n and a CDR3H region of SEQ ID NO: 309+n, wherein n is a number selected from the group consisting of 0 to 76.

The invention provides preferably an antibody according to the invention, characterized in that the antibody comprises a VL region selected from the group of VL regions comprising a CDR1L region of SEQ ID NO: 386+n, a CDR2L region of SEQ ID NO: 463+n and a CDR3L region of SEQ ID NO: 540+n, wherein n is a number selected from the group consisting of 0 to 76.

The invention provides preferably an antibody according to the invention, characterized in that the antibody comprises a VH region selected from the group of VH regions comprising a CDR1H region of SEQ ID NO: 155+n, a CDR2H region of SEQ ID NO:232+n and a CDR3H region of SEQ ID NO:309+n and in that the antibody comprises a VL region selected from the group of VL regions comprising a CDR1L region of SEQ ID NO:386+n, a CDR2L region of SEQ ID NO: 463+n and a CDR3L region of SEQ ID NO:540+n, wherein n is a number selected from the group consisting of 0 to 76.

The invention provides preferably an antibody according to the invention, characterized in comprising a VH region and a VL region comprising the respective CDR1, CDR2 and CDR3 regions of an antibody selected from the group consisting of antibodies P013.S.01.B.B03, P013.S.01.B.A05, P013.S.01.B.C04, P013.S.01.B.H01, P013.S.01.B.D03, P013.S.01.B.E02, P013.S.02.B.A04, P013.S.02.B.A05, P013.S.02.B.A02, P013.S.02.B.D03, P013.S.02.B.H01, P013.S.02.B.F01, P013.S.02.B.B04, P013.S.02.B.C02, P013.S.02.B.B05, P013.S.02.B.A03, P013.S.02.B.H03, and P013.S.02.B.G05.

The invention provides preferably an antibody according to the invention, characterized in comprising a VH region and a VL region comprising the respective CDR1, CDR2 and CDR3 regions of an antibody selected from the group consisting of antibodies P013.S.01.B.B03, P013.S.01.B.A05, P013.S.01.B.C04, P013.S.01.B.H01, P013.S.01.B.D03, P013.S.01.B.E02, P013.S.02.B.A04, P013.S.02.B.A05, P013.S.02.B.A02, P013.S.02.B.D03, P013.S.02.B.H01, P013.S.02.B.F01, P013.S.02.B.B04, P013.S.02.B.C02, P013.S.02.B.B05, P013.S.02.B.A03, P013.S.02.B.H03, and P013.S.02.B.G05.

The invention preferably provides an antibody specifically binding to human IL-1RAcP characterized in inhibiting IL-1RAcP induced NFkB activity, binding to the same epitope as an antibody selected from the group of antibodies P013.S.01.B.B03, P013.S.01.B.A05, P013.S.01.B.C04, P013.S.01.B.H01, P013.S.01.B.D03, P013.S.01.B.E02, P013.S.02.B.A04, P013.S.02.B.A05, P013.S.02.B.A02, P013.S.02.B.D03, P013.S.02.B.H01, P013.S.02.B.F01, P013.S.02.B.B04, P013.S.02.B.C02, P013.S.02.B.B05, P013.S.02.B.A03, P013.S.02.B.H03, and P013.S.02.B.G05,

The invention provides preferably an antibody according to the invention, characterized in being a monoclonal rabbit, rabbit/human chimeric or humanized rabbit antibody.

The invention provides a method for the production of a monoclonal rabbit antibody against human IL-1RAcP characterized in inhibiting IL1beta stimulated NFkB activity according to the invention, characterized in
i) that after immunizing said rabbit with IL-1RAcP, a number of antibody producing single cells derived from said rabbit are isolated,
ii) binding to IL-1RAcP is measured separately for the supernatants of said single cells,
iii) a single cell is selected if its supernatant shows binding to human IL-1RAcP and murine, and inhibits NFkB activity stimulated by IL-1alpha, IL-1beta, IL-33 and/or IL-36,
iv) an antibody with the properties of iii) is isolated from said selected cell.

Preferably the rabbit antibody producing single cell is a single B rabbit hybridoma cell.

The invention provides a method for the production of a monoclonal rabbit antibody binding to human IL-1RAcP, and inhibits NFkB activity stimulated by IL-1alpha, IL-1beta, IL-33 and/or IL-36

The invention provides a method for the production of a monoclonal rabbit antibody according to the invention, characterized in that after immunizing said rabbit with said antigen, a single antibody producing cell, preferably from a B cell is isolated from said animal or a rabbit hybridoma cell derived from said rabbit, is isolated, for which binding to human IL-1RAcP, and inhibition of NFkB activity stimulated by IL-1alpha, IL-1beta, IL-33 and/or IL-36, is found according to the invention.

The invention preferably provides the use of an antibody according to the invention for the manufacture of a pharmaceutical composition.

The invention provides a supernatant of a rabbit antibody producing single cell, preferably a single B cell or a rabbit hybridoma cell, characterized in binding to human IL-1RAcP, and inhibition NFkB activity stimulated by IL-1alpha, IL-1beta, IL-33 and/or IL-36, according to the invention.

The invention preferably provides a supernatant of a rabbit antibody producing single cell, preferably a single B cell or a rabbit hybridoma cell according to the invention, characterized in binding to human IL-1RAcP, and inhibition NFkB activity stimulated by IL-1alpha, IL-1beta, IL-33 and/or IL-36 according to the invention, binding to human IL-1RAcP, and inhibition NFkB activity stimulated by IL-1alpha, IL-1beta, IL-33 and/or IL-36 is measured for the supernatant of said cell and said antibody is isolated from said cell if it shows the properties according to the invention.

The invention provides a method for the production of a monoclonal rabbit antibody according to the invention, characterized in
i) that after immunizing said rabbit with said target antigen, a number of antibody producing single cells derived from said rabbit are isolated,
ii) binding to IL-1RAcP is measured separately for the supernatants of said single cells,
iii) a single cell is selected if its supernatant shows binding to human IL-1RAcP and murine, and inhibits NFkB activity stimulated by IL-1alpha, IL-1beta, IL-33 and/or IL-36 according to the invention,
iv) and an antibody is isolated from said selected cell if the antibody shows the properties according to iii).

Preferably the antigen used for immunization (IL-1RAcP) is a fusion polypeptide consisting of said antigen and a human Fc polypeptide. Preferably in step i) CFA is used as adjuvant. Preferably in step i) CFA and IFA are used together as adjuvants.

Preferably in step ii) B cells are isolated from the blood of the rabbit. B cells are isolated preferably as PBMCs and depleted from macrophages. The antigens used for isolating B cells in step iv) is the target proteins IL-1RAcP or a functional fragment thereof, preferably the extracellular domain or parts thereof, cells presenting the antigens on their surface or the like.

Preferably in step iii) single B cells, secreting immunoglobulin, preferably IgG, are separated, preferably by FACS. Preferably the single B cell is then treated with a feeder cell before performing step vi).

Preferably in step iii) single B cells are separated, characterized in secreting an antibody specifically binding to human IL-1RAcP and inhibiting IL-1RAcP induced NFkB activity. Preferably in step iii) single B cells are separated, characterized in secreting an antibody specifically binding to human and murine IL-1RAcP and inhibiting in addition murine IL-1RAcP induced murine NFkB activity.

Preferably in step iii) single B cells are separated, characterized in secreting an antibody against human IL-1RAcP characterized in inhibiting NFkB activity stimulated by IL-1alpha, IL-1beta, IL-33 and/or IL-36. Preferably in step iii) single B cells are separated, characterized in secreting an antibody against human IL-1RAcP inhibiting IL1alpha stimulated NFkB activity. Preferably in step iii) single B cells are separated, characterized in secreting an antibody against human IL-1RAcP and inhibiting IL1beta stimulated NFkB activity.

Preferably in step iii) single B cells are separated, characterized in secreting an antibody against human IL-1RAcP and inhibiting IL33 stimulated NFkB activity. Preferably in step iii) single B cells are separated, characterized in secreting an antibody against human IL-1RAcP and inhibiting IL36 stimulated NFkB activity.

Preferably in step iii) single B cells are separated, characterized in secreting an antibody against human IL-1RAcP and inhibiting NFkB activity stimulated by a complex selected from the group consisting of IL-1β/IL-1R1/IL-1RAcP, IL-1α/IL-1R1/IL-1RAcP IL-33/ST2/IL-1RAcP, and IL-36/Il-36R/IL-1RAcP.

Preferably in step iii) single B cells are separated, characterized in secreting an antibody binding to murine IL-1RAcP and inhibiting murine IL-1RAcP induced murine NFkB activity.

Preferably in step iii) single B cells are separated, characterized in secreting an antibody inhibiting in a concentration of 5 μg/ml (rabbit IgG isotype has a molecular weight of 150 KD) NFkB activity in 293T/17 cell lysates (293T/17 [HEK 293T/17] (ATCC® CRL-11268™)) stimulated with 0.5 μg/ml human IL-1alpha, IL-1beta, IL-33 and/or IL-36 (molecular weight see UniProtKB/Swiss-Prot), for 70% or more, preferably for 80% or more, preferably for 90% and more, and more preferably for 95% or more, related to the same assay without said antibody according to the invention.

Preferably in step iii) single B cells are separated, characterized in secreting an antibody inhibiting in a concentration of 5 μg/ml NFkB activity in respective mouse cell line lysates stimulated with 0.5 μg/ml murine IL-1alpha, IL-1beta, IL-33 and/or IL-36 (molecular weight see UniProtKB/Swiss-Prot), for 70% or more, preferably for 80% or more, preferably for 90% and more, and more preferably for 95% or more, related to the same assay without said antibody according to the invention.

Preferably in step iii) single B cells are separated, characterized in secreting an antibody stimulated with mol/l IL-1alpha, IL-1beta, IL-33 and/or IL-36, like antibody XX, or more in 293T/17 cells transfected with luciferase under control of NF-kB reporter gene).

Preferably the method according to the invention is characterized in selecting in step iii) a single B cell which comprises mRNA encoding a VH region of an antibody which binds specifically to human IL-1RAcP.

Preferably the antibody is a rabbit monoclonal antibody.

Preferably the antibody produced by the single B cell is tested, preferably by ELISA, whether it binds specifically to the respective antigens.

Preferably the antibody is tested whether it binds specifically to IL-1RAcP and selected if it binds. Preferably the antibody is produced recombinantly based on its nucleic acid and/or polypeptide sequence.

Preferably in step iii) a single B cell is selected which comprises mRNA encoding a VH region of a IL-1RAcP specific antibody as specified in FIG. 2, which is at least 90% identical to a VH region of SEQ ID NO: 1+n and mRNA encoding a VL region of an antibody specifically binding to IL-1RAcP, which is at least 90% identical to a VL region of SEQ ID NO:78+n, wherein n is a number selected from the group of 0 to 76.

“n is a number selected from the group of 0 to 76” according to the invention means a number selected from the group of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 51, 52, 53, 54, 55, 56, 57, 58, 59, 60, 61, 62, 63, 64, 65, 66, 67, 68, 69, 70, 71, 72, 73, 74, 75, and 76. The number “n” according to the invention is meant to be identical for the same antibody, its heavy and light chains, its variable regions and CDR regions.

The invention comprises a monoclonal antibody, characterized in specifically binding to comprising amino acid sequences as described herein.

The heavy chain variable (VH) region of a IL-1RAcP specific antibody is preferably characterized in that said VH region is at least 90% identical to a VH region selected from the group consisting of VH regions of SEQ ID NO: 1 to 77. The light chain variable (VL) region of a HER specific antibody is preferably characterized in that said VL region is at least 90% identical to a VL region selected from the group consisting of VL regions of SEQ ID NO: 78 to 154. The antibody according to the invention is preferably characterized in that its VH region is at least 90% identical to a VH region of SEQ ID NO: 1+n and its VL region is at least 90% identical to a VL region of SEQ ID NO: 78+n, wherein n is a number selected from the group consisting of 0 to 76. The antibody according to the invention is preferably characterized in that its VH region is selected from the group consisting of VH regions of SEQ ID NO: 1+n and its VL region is selected from the group consisting of VL regions of SEQ ID NO: 78+n, wherein n is a number selected from the group consisting of 0 to 76. The antibody according to the invention is preferably characterized in comprising a VH region and a VL region comprising the respective CDR1, CDR2 and CDR3 regions of an antibody selected from the group consisting of antibodies listed in FIG. 2. The antibody according to the invention is preferably characterized in that the antibody comprises a VH region selected from the group of VH regions comprising a CDR1H region of SEQ ID NO: 155+n, a CDR2H region of SEQ ID NO: 232+n and aCDR3H region of SEQ ID NO: 309+n, wherein n is a number selected from the group consisting of 0 to 76.

The antibody according to the invention is preferably characterized in that the antibody comprises a VL region selected from the group of VL regions comprising a CDR1L region of SEQ ID NO: 386+n, a CDR2L region of SEQ ID NO: 463+n and aCDR3L region of SEQ ID NO: 540+n, wherein n is a number selected from the group consisting of 0 to 76.

The antibody according to the invention is preferably characterized in that the antibody comprises a VH region selected from the group of VH regions comprising a CDR1H region of SEQ ID NO: 155+n, CDR2H region of SEQ ID NO: 232+n and aCDR3H region of SEQ ID NO:309+n, and in that the antibody comprises a VL region selected from the group of VL regions comprising a CDR1L region of SEQ ID NO: 386+n, a CDR2L region of SEQ ID NO: 463+n and a CDR3L region of SEQ ID NO: 540+n, wherein n is a number selected from the group consisting of 0 to 76.

The invention provides also compositions, B cells, methods of use, and methods of production of the antibodies according to the invention.

The antibody according to the invention is preferably characterized in being a humanized or chimeric version of said antibody. Preferably, the antibody according to the invention is an antibody comprising antigen binding sequences from a rabbit donor grafted to a heterologous non-human, human, or humanized sequence (e.g., framework and/or constant domain sequences). Preferably, an antibody of the invention has rabbit V regions or rabbit CDR regions and a human C region and/or framework. Preferably, the rabbit VL region or a human framework region comprising rabbit light chain CDRs is fused to a human kappa light chain constant region. Preferably, the rabbit VH region or a human framework region comprising rabbit heavy chain CDRs is fused to a human constant region, preferably IgG1. Preferably the invention relates to a chimeric or humanized rabbit antibody, characterized in comprising serine instead of the cysteine which is located at a position between amino acid 75 to 85 in the variable light chain VL.

The invention also provides a pharmaceutical composition characterized by comprising an antibody according to the invention. The invention also provides the use of an antibody according to the invention for the manufacture of a pharmaceutical composition. The invention also provides an antibody according to the invention for the treatment of a patient in the need of such treatment, preferably in the treatment of cancer. The invention also provides an antibody according to the invention for the treatment of breast, colon, lung, or pancreatic cancer. The invention also provides the use of an antibody according to the invention for manufacture of a medicament for the treatment of a patient in the need of such treatment, preferably in the treatment of cancer. The invention also provides the use of an antibody according to the invention for manufacture of a medicament for the treatment of breast, colon, lung, or pancreatic cancer. The invention also provides an antibody according to the invention for use in the treatment of a patient in the need of such treatment, preferably in the treatment of cancer, preferably in the treatment of breast, colon, lung, or pancreatic cancer.

The invention also provides a nucleic acid encoding an antibody according to the invention. The invention also provides an expression vector characterized in comprising a nucleic acid according to the invention for the expression of an antibody according to the invention in a prokaryotic or eukaryotic host cell. The invention also provides a prokaryotic or eukaryotic host cell comprising a nucleic acid according to the invention. The invention also provides a method of producing an antibody according to the invention characterized by expressing a nucleic acid according to the invention in a prokaryotic or eukaryotic host cell and recovering said antibody from said cell or the cell culture supernatant.

Preferably the antibodies of the present invention are antagonistic antibodies.

Sequences of said antibodies, antibodies comprising said VH and/or VL regions or said CDR regions are shown in FIG. 2.

### DETAILED DESCRIPTION OF THE INVENTION

The term “rabbit” according to the invention means an animal of the members of the taxonomic order Lagomorpha, which includes the families (hares and rabbits) and Ochotonidae (pikas), preferably of genus Oryctolagus.

The term “antibody” encompasses the various forms of antibody structures including, but not being limited to, whole antibodies and antibody fragments as long as it shows the properties according to the invention. The antibody according to the invention is in its primary form produced by a B-cell of a rabbit and binds to IL-1RAcP. Therefore, the antibody according to the invention binds specifically to IL-1RAcP based on its antigen-binding portion, preferably its VH region comprising three VH CDRs and/or its VL region comprising three VL CDRs.

The term “rabbit monoclonal antibody” according to the invention means a monoclonal antibody produced by immunizing a rabbit and isolated from an antigen producing cell of said rabbit as well as such an antibody which is further modified, preferably a humanized antibody, a chimeric antibody, a fragment thereof, or a further genetically engineered and recombinant produced antibody as long as the characteristic properties according to the invention are retained. Preferably the antibody is from a B cell or a rabbit hybridoma cell of said rabbit.

The term “antibody producing cell” according to the invention means a rabbit B cell which produce antibodies, preferably a B cell or rabbit hybridoma cell.

“Native antibodies” are usually heterotetrameric glycoproteins composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “VL (or VH) region” has the same meaning as VL (or VH) domain. The antibody according to the invention is in its primary form a mature antibody, which may be different from a simple germline antibody. Without being bound by theory, it is believed that binding of the antigen to a germline antibody might lead to significant structural rearrangements, whereas the unbound state of a matured antibody might be closer to its bond state. Therefore, the mature form of the antibody has probably a more rigid structure than the germline form. The germline antibody might be therefore more conformational flexible, resulting in a slower binding rate (see e.g. Wedemayer G J et al., Science. 1997 Jun. 13; 276(5319):1665-9; Structural insights into the evolution of an antibody combining site). The presumably lower flexible structure of the mature antibody may improve the physicochemical properties of the antibody according to the invention, as being e.g. solubility or low aggregation, leading to improved therapeutic properties. The antibody according to the invention as identified from a rabbit B cell is an antibody having variable regions of natural origin.

“Natural origin” means according to the invention, that such an antibody has variable regions which are identical in their amino acid sequences to the sequences of variable regions naturally occurring in rabbits. The antibody according to the invention can be further modified and is preferably a rabbit antibody, a humanized antibody, a chimeric antibody, a fragment thereof, or a further genetically engineered and recombinant produced antibody as long as the characteristic properties according to the invention are retained. The antibody can be bound to a further agent, e.g. as being an immunoconjugate. Preferably the antibody according to the invention is a rabbit antibody.

Preferably the antibody in its primary form binds specifically to human IL-1RAcP and murine IL-1RAcP.

The term “supernatant of a single cell” according to the invention means the supernatant of the culture of a rabbit antibody producing single cell, preferably a B cell or a rabbit hybridoma cell. Such supernatant comprises a monoclonal antibody according to the invention. The Fc part/constant part is therefore in a naturally occurring glycosylation condition.

The terms “Fc receptor” or “FcR” according to the invention refers to a human receptor that binds to the Fc region of an antibody. FcRs bind IgG antibodies and include receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daeron, Annu. Rev. Immunol. 15:203-234 (1997)). FcRIIIA (CD16a) mediaties ADCC. FcRs are reviewed in Ravetch and Kinet, Annu. Rev. Immunol 9:457-92 (1991); Capel et al, Immunomethods 4:25-34 (1994); and de Haas et al, J. Lab. CHn. Med. 126:330-41 (1995). These and all other FcRs are encompassed by the term “FcR” herein. The term also includes the neonatal receptor, FcRn, which is responsible for the transfer of maternal IgGs to the fetus (Guyer et al, J. Immunol. 117:587 (1976) and Kim et al, J. Immunol. 24:249 (1994)) and mediates slower catabolism, thus longer half-life.

The “constant domains (constant parts)” are not involved directly in binding of an antibody to an antigen, but exhibit e.g. also effector functions. The heavy chain constant region that corresponds to human IgG1 is called γ1 chain. The heavy chain constant region that correspond to human IgG3 is called γ3 chain. Human constant γ heavy chains are described in detail by Kabat, E. A. et al., Sequences of Proteins of Immunological Interest, 5th ed., Public Health Service, National Institutes of Health, Bethesda, MD. (1991), and by Brueggemann, M., et al., J. Exp. Med. 166 (1987) 1351-1361; Love, T. W., et al., Methods Enzymol. 178 (1989) 515-527. Constant domains of IgG1 or IgG3 type are glycosylated at Asn297. “Asn 297” according to the invention means amino acid asparagine located at about position 297 in the Fc region; based on minor sequence variations of antibodies, Asn297 can also be located some amino acids (usually not more than +3 amino acids) upstream or downstream.

Glycosylation of human IgG1 or IgG3 occurs at Asn297 as core fucosylated bianntennary complex oligosaccharide glycosylation terminated with up to 2 Gal (galactose) residues. These structures are designated as G0, G1 (α1,6 or α1,3) or G2 glycan residues, depending from the amount of terminal Gal residues (Raju, T. S., BioProcess International 1 (2003) 44-53). CHO type glycosylation of antibody Fc parts is e.g. described by Routier, F. H., Glycoconjugate J. 14 (1997) 201-207. Cell-mediated effector functions like ADCC of antibodies according to the invention can be further enhanced by engineering the oligosaccharides attached at the Fc region of the antibody (defucosylation) as described in Umana, P., et al, Nature Biotechnol. 17 (1999) 176-180, Naoko Yamane-Ohnuki and Mitsuo Satoh, MAbs. 2009; 1(3): 230-236 and U.S. Pat. No. 6,602,684, WO 2005/044859, WO 2004/065540, WO2007/031875. Such methods are e.g. use of the host cells with reduced intrinsic α-1,6 fucosylation ability, e.g., Lec13, a variant of CHO cells partially deficient in GMD function, or YB2/0, a rat-rat hybridoma cell line with intrinsically reduced FUT8 activity; introduction of small interfering RNA (siRNA) against the α-1,6 fucosylation relevant genes; co-introduction of β-1,4-N-acetylglucosaminyltransferase (GnTIII) and Golgi α-mannosidase II (ManII);58,83,84 and disruption of the genomic locus responsible for α-1,6 fucosylation.

The term “antibody effector function(s),” or “effector function” as used herein refers to a function contributed by an Fc effector domain(s) of an IgG (e.g., the Fc region of an immunoglobulin). Such function can be effected by, for example, binding of an Fc effector domain(s) to an Fc receptor on an immune cell with phagocytic or lytic activity or by binding of an Fc effector domain(s) to components of the complement system. Typical effector functions are ADCC, ADCP and CDC. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

“Antibody-dependent cell-mediated cytotoxicity” and “ADCC” refer to a cell-mediated reaction in which nonspecific cytotoxic cells that express FcRs (e.g. Natural Killer (NK) cells, neutrophils, and macrophages) recognize bound antibody on a target cell and subsequently cause lysis of the target cell. The primary cells for mediating ADCC, NK cells, express FcyRIII only, whereas monocytes express FcyRI, FcyRII and FCYRIII. FCR expression on hematopoietic cells is summarized in Table 3 on page 464 of Ravetch, and Kinet, Annu. Rev. Immunol 9 (1991) 457-492. The term “Antibody-dependent cellular phagocytosis” and “ADCP” refer to a process by which antibody-coated cells are internalized, either in whole or in part, by phagocytic immune cells (e.g., macrophages, neutrophils and dendritic cells) that bind to an immunoglobulin Fc region.

C1q″ is a polypeptide that includes a binding site for the Fc region of an immunoglobulin. C1q together with two serine proteases, C1r and C1s, forms the complex C1, the first component of the complement dependent cytotoxicity (CDC) pathway. Human C1q can be purchased commercially from, e.g. Quidel, San Diego, Calif.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgGi, IgG₂, IgG₃, IgG₄, IgAi, and IgA₂. The heavy chain constant domains that correspond to the different classes of immunoglobulins are called a, δ, ε, γ, and μ, respectively.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis (ADCP); down regulation of cell surface receptors (e.g. B cell receptor); and B cell activation. A “reduced effector function” as used herein refers to a reduction of a specific effector function, like for example ADCC or CDC, in comparison to a control (for example a polypeptide with a wildtype Fc region), by at least 20% and a “strongly reduced effector function” as used herein refers to a reduction of a specific effector function, like for example ADCC or CDC, in comparison to a control, by at least 50%.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat, et al., Sequences of Proteins of Immunological Interest, 5th Ed. Public Health Service, National Institutes of Health, Bethesda, MD (1991). A “variant Fc region” comprises an amino acid sequence which differs from that of a “native” or “wildtype” sequence Fc region by virtue of at least one “amino acid modification” as herein defined. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

The term “Fc-variant” as used herein refers to a polypeptide comprising a modification in an Fc domain. The Fc variants of the present invention are defined according to the amino acid modifications that compose them. Thus, for example, P329G is an Fc variant with the substitution of proline with glycine at position 329 relative to the parent Fc polypeptide, wherein the numbering is according to the EU index. The identity of the wildtype amino acid may be unspecified, in which case the aforementioned variant is referred to as P329G. For all positions discussed in the present invention, numbering is according to the EU index. The EU index or EU index as in Kabat or EU numbering scheme refers to the numbering of the EU antibody (Edelman, et al., Proc Natl Acad Sci USA 63 (1969) 78-85, hereby entirely incorporated by reference.) The modification can be an addition, deletion, or substitution. Substitutions can include naturally occurring amino acids and non-naturally occurring amino acids. Variants may comprise non-natural amino acids. Examples include U.S. Pat. No. 6,586,207; WO 98/48032; WO 03/073238; US 2004/0214988 AI; WO 05/35727A2; WO 05/74524 A2; Chin, J. W., et al., Journal of the American Chemical Society 124 (2002) 9026-9027; Chin, J. W., and Schultz, P. G., ChemBioChem 11 (2002) 1135-1137; Chin, J. W., et al., PICAS United States of America 99 (2002) 11020-11024; and, Wang, L., and Schultz, P. G., Chem. (2002) 1-10, all entirely incorporated by reference.

The term “Fc region-containing polypeptide” refers to a polypeptide, such as an antibody or immunoadhesin (see definitions below), which comprises an Fc region.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcyRI, FcyRII, and FcyRIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain, (see review in Daeron, M., Annu. Rev. Immunol. 15 (1997) 203-234). FcRs are reviewed in Ravetch, and Kinet, Annu. Rev. Immunol 9 (1991) 457-492; Capel, et al., Immunomethods 4 (1994) 25-34; and de Haas, et al., J. Lab. Clin. Med. 126 (1995) 330-41. Other FcRs, including those to be identified in the future, are encompassed by the term “FcR” herein. The term also includes the neonatal receptor, FcRn, which is responsible for the transfer of maternal IgGs to the fetus (Guyer, et al., J. Immunol. 117 (1976) 587 and Kim, et al., J. Immunol. 24 (1994) 249).

By “IgG Fc ligand” as used herein is meant a molecule, preferably a polypeptide, from any organism that binds to the Fc region of an IgG antibody to form an Fc/Fc ligand complex. Fc ligands include but are not limited to FcyRs, FcyRs, FcyRs, FcRn, Clq, C3, mannan binding lectin, mannose receptor, staphylococcal protein A, streptococcal protein G, and viral FcyR. Fc ligands also include Fc receptor homologs (FcRH), which are a family of Fc receptors that are homologous to the FcyRs (Davis, et al., Immunological Reviews 190 (2002) 123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcyR” or “FcgammaR” as used herein is meant any member of the family of proteins that bind the IgG antibody Fc region and is encoded by an FcyR gene. In humans this family includes but is not limited to FcyRI (CD64), including isoforms FcyRIA, FcyRIB, and FcyRIC; FcyRII (CD32), including isoforms FcyRIIA (including allotypes H131 and R131), FcyRIIB (including FcyRIIB-I and FcyRIIB-2), and FcyRIIc; and FcyRIII (CD 16), including isoforms FcyRIIIA (including allotypes VI 58 and F158) and FcyRIIIb (including allotypes FcyRIIB-NAI and FcyRIIB-NA2) (Jefferis, et al., Immunol Lett 82 (2002) 57-65, entirely incorporated by reference), as well as any undiscovered human FcyRs or FcyR isoforms or allotypes. An FcyR may be from any organism, including but not limited to humans, mice, rats, rabbits, and monkeys. Mouse FcyRs include but are not limited to FcyRI (CD64), FcyRII (CD32), FcyRIII (CD 16), and FCYRIII-2 (CD 16-2), as well as any undiscovered mouse FcyRs or FcyR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin.

The term “IL-1RAcP specific antibody”, as used herein refers to an antibody specifically to human IL-1RAcP. “IL-1RAcP specific antibody” in conjunction with the VH, VL and CDR sequences specified in example 1 denotes an antibody with the specificity shown in FIG. 2. Therefore and for example a “IL-1RAcP specific antibody, characterized in that its VH region is selected from the group consisting of VH regions of SEQ ID NO:1+n and its VL region is selected from the group consisting of VL regions of SEQ ID NO:37+n, wherein n is a number from 0 to 3” means “an antibody selected from the group consisting of the IL-1RAcP specific antibodies, characterized by a VH region of SEQ ID NO:1 and a VL region of SEQ ID NO:37, by a VH region of SEQ ID NO:2 and a VL region of SEQ ID NO:38, by a VH region of SEQ ID NO: 4 and a VL region of SEQ ID NO:40, and of the IL-1RACP specific antibody, characterized by a VH region of SEQ ID NO:3 and a VL region of SEQ ID NO:39.

An “immunoconjugate” means an antibody conjugated to one or more cytotoxic agents, such as a chemotherapeutic agent, a drug, a growth inhibitory agent, a toxin, another antibody or a radioactive isotope.

“Antibody fragments” comprise a portion of a full length antibody, preferably the variable regions thereof, or at least the antigen binding site thereof. Examples of antibody fragments include diabodies, Fab fragments, and single-chain antibody molecules. scFv antibodies are, e.g., described in Huston, J. S., Methods in Enzymol. 203 (1991) 46-88.

The terms “monoclonal antibody” or “monoclonal antibody composition” as used herein refer to a preparation of antibody molecules of a single amino acid composition. The term “chimeric antibody” refers to a monoclonal antibody comprising a variable region, i.e., binding region, from rabbit and at least a portion of a constant region derived from a different source or species, usually prepared by recombinant DNA techniques. According to the invention chimeric antibodies comprising a rabbit variable region and a human constant region and humanized rabbit antibodies are especially preferred. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art (see, e.g., Morrison, S. L., et al, Proc. Natl. Acad. Sci. USA 81 (1984) 6851-6855; U.S. Pat. Nos. 5,202,238 and 5,204,244).

The term “humanized antibody” or “humanized version of an antibody” refers to antibodies in which a human variable region has been modified to comprise the CDRs of an antibody according to the invention. In a preferred embodiment, the CDRs of the VH and VL are grafted into the framework region of human antibody to prepare the “humanized antibody.” See e.g. Riechmann, L., et al, Nature 332 (1988) 323-327; and Neuberger, M. S., et al, Nature 314 (1985) 268-270. The heavy and light chain variable framework regions can be derived from the same or different human antibody sequences. The human antibody sequences can be the sequences of naturally occurring human antibodies. Human heavy and light chain variable framework regions are listed e.g. in Lefranc, M.-P., Current Protocols in Immunology (2000)—Appendix IP A.1P.1-A.1P.37 and are accessible via IMGT, the international ImMunoGeneTics information system® (http://imgt.cines.fr) or via http://vbase.mrc-cpe.cam.ac.uk.

Preferably the invention relates to a chimeric or humanized rabbit antibody, characterized in comprising serine instead of the cysteine which is located at a position between amino acid 75 to 85 in the variable light chain VL.

The term “recombinant antibody”, as used herein, is intended to include all antibodies according to the invention that are prepared by recombinant means, such as antibodies from a host cell such as a NSO or CHO cell using a recombinant expression vector transfected into a host cell. Such recombinant human antibodies have variable and constant regions in a rearranged form.

The terms “specifically binding, against target, or anti-target antibody”, as used herein, refer to binding of the antibody to the respective antigen (target), measured by ELISA, wherein said ELISA preferably comprises coating the respective antigen to a solid support, adding said antibody under conditions to allow the formation of an immune complex with the respective antigen or protein, detecting said immune complex by measuring the Optical Density values (OD) using a secondary antibody binding to an antibody according to the invention and using a peroxidase-mediated color development. The term “antigen” according to the invention refers to the antigen used for immunization or a protein comprising said antigen as part of its protein sequence. For example, for immunization a fragment of the extracellular domain of a protein (e.g. the first 20 amino acids) can be used and for detection/assay and the like the extracellular domain of the protein or the full length protein can be used.

The term “specifically binding” or “specifically recognized” herein means that an antibody exhibits appreciable affinity for an antigen and, preferably, does not exhibit significant crossreactivity. “Appreciable” binding affinity includes binding with an affinity of at least 10exp7M^{31 1}, specifically at least 10exp8M⁻¹, more specifically at least 10exp9M⁻¹, or even yet more specifically at least 10exp10M⁻¹. An antibody that “does not exhibit significant crossreactivity” is one that will not appreciably bind to an undesirable other protein. An antibody specific for an epitope according to the invention will, for example, not significantly crossreact with other epitopes on IL-1RAcP. Specific binding can be determined according to any art-recognized means for determining such binding. In some embodiments, specific binding is determined by competitive binding assays (e.g. ELISA). The term “inhibiting IL-1RAcP induced NFkB activity” as used herein refers to inhibition of NFkB activity in a luciferase reporter experiment. 293T/17 [HEK 293T/17] (ATCC® CRL-11268™) cells, which express a NF-kB-RE firefly luciferase reporter, are seeded into Poly-D-Lysin-Cell culture plates. After stimulation of of IL-1RAcP the cell lysate is tested for activated NF-kB using the Steady-Glo® Luciferase Assay Kit (Promega Corp. Madison USA). Supernatants with functional antibodies bind to IL-1RAcP and inhibit the NF-kB activation, which is shown in low signal. The Steady-Glo® Luciferase Assay Kit is described in https://www.promega.de/resources/protocols/technical-manuals/0/steady-glo-luciferase-assay-system-protocol and Alam, J. and Cook, J. L. (1990) Anal. Biochem. 188, 245-54; Wood, K. V. (1991) In: Bioluminescence and Chemiluminescence: Current Status, Stanley, P., and Kricka, L., eds., John Wiley and Sons, Chichester, NY, 543; Ow, D. W. et al. (1986). Science 234, 856-9; De Wet, J. R. et al. (1987) Mol. Cell. Biol. 7, 725-37; Wood, K. V. (1990) PromegaNotes 28, 1-3; Wood, K. V. (1991) In: Bioluminescence and Chemiluminescence: Current Status, Stanley, P. and Kricka, L., eds., John Wiley and Sons, Chichester, NY, 11; and U.S. Pat. Nos. 5,283,179, 5,641,641, 5,650,289.

The antibody according to the invention comprises a VH region and a VL region or parts thereof, which are both together sufficient for the specific binding to the respective antigen.

All protein terms as used herein refers to the human proteins. If a protein from another species is meant, this is explicitly mentioned.

The term “IL-1RAcP””, as used herein, refers to human IL-1RAcP (UniProtKB Q9NPH3), which is a Coreceptor for IL1RL2 in the IL-36 signaling system (By similarity). Coreceptor with IL1R1 in the IL-1 signaling system. Associates with IL1R1 bound to IL1B to form the high affinity interleukin-1 receptor complex which mediates interleukin-1-dependent activation of NF-kappa-B and other pathways (UniProtKB). The term “murine IL-1RAcP””, as used herein, refer to murine IL-1RAcP (UniProtKB Q61730).

The term “IL-1alpha””, as used herein, refers to human IL-1 (UniProtKB P01583). The term “IL-1beta””, as used herein, refer to human IL-1beta (UniProtKB P01584). IL-1 stimulates thymocyte proliferation by inducing IL-2 release, B-cell maturation and proliferation, and fibroblast growth factor activity. IL-1 proteins are involved in the inflammatory response, being identified as endogenous pyrogens (UniProtKB).

The term “IL-33””, as used herein, refers to human IL-33 (UniProtKB 095760), acytokine that binds to and signals through the IL1RL1/ST2 receptor which in turn activates NF-kappa-B and MAPK signaling pathways in target cells (UniProtKB).

The term “IL-36””, as used herein, refers to human IL-36alpha (UniProtKB Q9UHA7, IL-36beta (UniProtKB Q9NZH7) and or IL-36gamma (UniProtKB Q9NZH8). IL-36 are cytokines that bind to and signal through the IL1RL2/IL-36R receptor which in turn activates NF-kappa-B and MAPK signaling pathways in target cells linked to a pro-inflammatory response. Part of the IL-36 signaling system that is thought to be present in epithelial barriers and to take part in local inflammatory response; similar to the IL-1 system with which it shares the coreceptor IL1RAP. IL-36 seems to be involved in skin inflammatory response by acting on keratinocytes, dendritic cells and indirectly on T cells to drive tissue infiltration, cell maturation and cell proliferation (UniProtKB).

The term “NFkB” as used herein, refer to human nuclear factor NF-kappa-B, which consists of p105 subunit (P19838) and p100 subunit (Q00653). “Inhibition of NFkB” is measured according to the invention as inhibition of NFkB dependent luciferase gene expression in human cells. Such methods are e.g. described in Windheim M. et al., Mol. Cell. Biol. 28 (2008) 1783-1791; Huang J. et al. PNAS USA 94 (1997) 12829-12832; Xiaoxia L. et al., Mol. Cell, Biol. 19 (1999) 4643-4652. The method used according to the invention as inhibition of IL1beta induced NFkB expression in 293T/17 cells is described in the example section of this patent application. If murine NFkB is meant herein it is explicitly mentioned.

The “variable region (or domain) of an antibody according to the invention” (variable region of a light chain (VL), variable region of a heavy chain (VH)) as used herein denotes each of the pair of light and heavy chain regions which are involved directly in binding the antibody to the antigen. The variable light and heavy chain regions have the same general structure and each region comprises four framework (FR) regions whose sequences are widely conserved, connected by three complementary determining regions, CDRs. The term “antigen-binding portion of an antibody” when used herein refer to the amino acid residues of an antibody which are responsible for antigen-binding. The antigen-binding portion of an antibody comprises preferably amino acid residues from the “complementary determining regions” or “CDRs”. The CDR sequences are defined according to Kabat et al, Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable region. For example, a heavy chain variable region may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. The variable domain of the heavy chain of an antibody according to the invention is composed of a single immunoglobulin domain and is about 110 to 120 amino acids long. The variable domain of the light chain of an antibody according to the invention is composed of a single immunoglobulin domain and is about 110 to 120 amino acids long.

In one embodiment the antibody according to the invention comprises a Fc part or constant heavy and light parts derived from human origin and preferably comprising all parts of the human constant regions. As used herein the term “Fc part derived from human origin” denotes a Fc part which is either a Fc part of a human antibody of the subclass IgG1, IgG2, IgG3 or IgG4, e.g. a Fc part from human IgG1 subclass, a mutated Fc part from human IgG1 subclass (preferably with a mutation on L234A+L235A), a Fc part from human IgG4 subclass or a mutated Fc part from human IgG4 subclass (preferably with a mutation on S228P). In one embodiment the antibody according to the invention is of human IgG1 subclass. Human constant chains are well known in the state of the art and e.g. described by Kabat, E. A., (see e.g. Johnson, G. and Wu, T. T., Nucleic Acids Res. 28 (2000) 214-218).

In one embodiment the antibody according to the invention comprises a heavy chain variable region (VH) sequence having at least 90%, 91%, 92%>, 93%>, 94%, 95%, 96%, 97%, 98%, 99%, or 100% sequence identity to an amino acid sequence selected from the group of VH sequences according to the invention. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, whereby the antibody retains the ability to bind specifically according to the invention to the respective antigen. In certain embodiments, a total of 1 to 10 amino acids have been substituted, inserted and/or deleted in each of said VH sequences. In certain embodiments, substitutions, insertions, or deletions occur in regions outside the CDRs (i.e., in the FRs).

In one embodiment the antibody according to the invention comprises a light chain variable region (VL) having at least 90%, 91%, 92%, 93%, 94%, 95%, 96%, 97%, 98%, 99%, or 100% sequence identity to the amino acid sequence of the VL sequences according to the invention, wherein n is a number from 0 to 5. In certain embodiments, a VL sequence having at least 90%, 91%, 92%, 93%, 94%, 95%, 96%, 97%, 98%, or 99% o identity contains substitutions (e.g., conservative substitutions), insertions, or deletions relative to the reference sequence, whereby the antibody retains the ability to bind specifically to the respective antigen. In certain embodiments, a total of 1 to 10 amino acids have been substituted, inserted and/or deleted in said VL sequences. In certain embodiments, the substitutions, insertions, or deletions occur in regions outside the CDRs (i.e., in the FRs). The invention also comprises affinity matured antibodies which can be produced according to methods known in the art. Marks et al. Bio/Technology 10:779-783 (1992) describes affinity maturation by VH and VL domain shuffling. Random mutagenesis of CDR and/or framework residues is described by: Barbas et al., Proc Nat. Acad. Sci, USA 91:3809-3813 (1994); Schier et al., Gene 169:147-155 (1995); Yelton et al., J. Immunol. 1 55:1994-2004 (1995); Jackson et al., J. Immunol. 1 54(7):3310-9 (1995); and Hawkins et al., J. Mol. Biol. 226:889-896 (1992) and WO2010108127. “Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software.

The antibodies according to the invention are preferably produced by recombinant means. Such methods are widely known in the state of the art and comprise protein expression in prokaryotic and eukaryotic cells with subsequent isolation of the antibody polypeptide and usually purification to a pharmaceutically acceptable purity. For the protein expression nucleic acids encoding light and heavy chains of an antibody according to the invention or fragments thereof are inserted into expression vectors by standard methods. Expression is performed in appropriate prokaryotic or eukaryotic host cells, such as CHO cells, NSO cells, SP2/0 cells, HEK293 cells, COS cells, yeast, or E. coli cells, and the antibody is recovered from the cells (from the supernatant or after cells lysis). Recombinant production of antibodies is well-known in the state of the art and described, for example, in the review articles of Makrides, S. C., Protein Expr. Purif. 17 (1999) 183-202; Geisse, S., et al, Protein Expr. Purif. 8 (1996) 271-282; Kaufman, R. J., Mol. Biotechnol. 16 (2000) 151-161; Werner, R. G., Drug Res. 48 (1998) 870-880. The antibodies may be present in whole cells, in a cell lysate, or in a partially purified, or pure form. Purification is performed in order to eliminate other cellular components or other contaminants, e.g., other cellular nucleic acids or proteins, by standard techniques, including, column chromatography and others well known in the art (see Ausubel, F., et al, ed. Current Protocols in Molecular Biology, Greene Publishing and Wiley Interscience, New York (1987)). Expression in NSO cells is described by, e.g., Barnes, L. M., et al, Cytotechnology 32 (2000) 109-123; Barnes, L. M., et al, Biotech. Bioeng. 73 (2001) 261-270. Transient expression is described by, e.g., Durocher, Y., et al, Nucl. Acids. Res. 30 (2002) E9. Cloning of variable domains is described by Orlandi, R., et al, Proc. Natl. Acad. Sci. USA 86 (1989) 3833-3837; Carter, P., et al, Proc. Natl. Acad. Sci. USA 89 (1992) 4285-4289; Norderhaug, L., et al, J. Immunol. Methods 204 (1997) 77-87. A preferred transient expression system (HEK 293) is described by Schlaeger, E.-J. and Christensen, K., in Cytotechnology 30 (1999) 71-83, and by Schlaeger, E.-J., in J. Immunol. Methods 194 (1996) 191-199. Monoclonal antibodies are suitably separated from the culture medium by conventional immunoglobulin purification procedures such as, for example, protein A-Sepharose, hydroxylapatite chromatography or affinity chromatography.

DNA and RNA encoding the monoclonal antibodies are sequenced using conventional procedures. RT PCR is preferably used.

Antibodies obtained from said cell lines are preferred embodiments of the invention. Amino acid sequence variants of an antibody are prepared by introducing nucleotide changes into the antibody encoding DNA, or by peptide synthesis. Any cysteine residue not involved in maintaining the proper conformation of the antibody may also be substituted, generally with serine, to improve the oxidative stability of the molecule and to prevent aberrant crosslinking. Conversely, cysteine bond(s) may be added to the antibody to improve its stability (particularly where the antibody is an antibody fragment such as an Fv fragment).

The heavy and light chain variable regions according to the invention are combined with sequences of promoter, translation initiation, constant region, 3′ untranslated region, polyadenylation, and transcription termination to form expression vector constructs. The heavy and light chain expression constructs can be combined into a single vector, co-transfected, serially transfected, or separately transfected into host cells which are then fused to form a single host cell expressing both chains.

One aspect of the invention is a pharmaceutical composition comprising an antibody according to the invention. Another aspect of the invention is the use of an antibody according to the invention for the manufacture of a pharmaceutical composition. A further aspect of the invention is a method for the manufacture of a pharmaceutical composition comprising an antibody according to the invention. In another aspect, the present invention provides a composition, e.g. a pharmaceutical composition, containing an antibody according to the present invention, formulated together with a pharmaceutical carrier.

Furthermore, the antibodies according to the invention are especially useful for the treatment of diseases where the dysregulation of the target is the underlying reason. One aspect of the invention is a pharmaceutical composition for the treatment of cancer.

Another aspect of the invention is an antibody according to the invention for the treatment of cancer. For this the antibody according to the invention can be investigated in a respective mouse tumor model e.g. according to Krupke D M; Begley D A; Sundberg J P; Bult C J; Eppig J T, The Mouse Tumor Biology database, Nat Rev Cancer 2008 June; 8(6):459-65. Therefore, one aspect of the invention is a pharmaceutical composition for the treatment of cancer.

Another aspect of the invention is an antibody according to the invention for the treatment of cancer.

Another aspect of the invention is the use of an antibody according to the invention for the manufacture of a medicament for the treatment of cancer.

Another aspect of the invention is a method of treatment of a patient suffering from cancer by administering an antibody according to the invention to said patient in the need of such treatment.

As used herein, “pharmaceutical carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion).

A composition of the present invention can be administered by a variety of methods known in the art. As will be appreciated by the skilled artisan, the route and/or mode of administration will vary depending upon the desired results. To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion.

The term “cancer” as used herein may be, for example, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, lymphocytic leukemia, including refractory versions of any of the above cancers, or a combination of one or more of the above cancers. Preferably such cancer is a breast cancer, colon cancer, lung cancer, or pancreatic cancer.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin. Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The method according to the invention comprises in summary the steps of immunization, B cell isolation, enrichment of B cells, isolation of single B cells, preferably co-cultivation with feeder cells, selection of a single B cell which comprises respective mRNA, and production of the antibody according to the invention. Such methods are mentioned for the production of monospecific antibodies e.g. in WO2011147903, WO2007003041, WO2008045140, WO2004106377, EP1255780, and EP1633787.

### Immunization

Immunization can be performed according to the methods known of the state of the art, e.g. by using DNA of the target antigens or fragments thereof, complete protein antigens or fragments thereof, antigen expressing cells. Preferably the IL-1RAcP antigen is a fusion polypeptide consisting of said antigen and a human Fc polypeptide. Preferably immunization in step i) is repeated at least three times and appropriately up to six times during 90 days (if an antibody according to the invention is identified already after e.g. the fourth immunization, further immunizations are not necessary). Preferably complete Freund's adjuvant (CFA) or CFA and incomplete Freund's adjuvant (IFA) is (are) used as adjuvant.

### B Cell Isolation

The B-cells are isolated from the rabbit, preferably from the blood of the rabbit. The B-cells are isolated up to 8 days, preferably 5 to 7 days, after 3rd to 6th immunization. Preferably PBMCs are isolated and depleted from macrophages (see e.g. EP0488470) and used as B cells. Isolation of B cells can be for example also performed by labeling non-B cells with non B cell markers, e.g. anti CD2, CD14, CD16, CD36, CD43, and CD235a antibodies and separating the labeled non B cells from non-labeled B cells.

### Enrichment of B Cells

Antibody producing and antigen specific B cells are preferably isolated (enriched) by treating the B cells with IL-1RAcP antigen used for immunization, or a cell expressing the respective antigen. Preferably the antigen and the cell expressing the antigen are used in immobilized manner, so that the antigen specific B cells can be separated easily. Such methods are e.g. described in Kodituwakko AP et al., Immunol. Cell Biol. (2003) 81, 163-170 and EP0488470.

### Isolation of Single B Cells

Isolation of single rabbit B cells is preferably performed by FACS. Preferably an anti-rabbit IgG, is used for FACS selection. Such selected single B cells are antibody producing B cells.

### Co-Cultivation With Feeder Cells

Preferably the antigen producing B cells are co-cultivated with feeder cells before the selection step (see below) is performed. Such a feeder cell is preferably a thymoma cell line such as the murine EL4 thymoma cell line, which is preferably mutagenized; preferably the thymoma cell line is mutagenized to a bromo-deoxyuridine-resistant mutant (e.g. EL4-B5 cells, Wen L. et al., Eur. J. Immunol. 17 (1987) 887-92,). This increases the amount of antibody in the cell supernant (see e.g. Zubler, R. H., et al., Eur. J. Immunol. 14 (1984) 357-63, Wen L. et al., Eur. J. Immunol. 17 (1987) 887-92, Hoffmann P et al., J Immunol. Methods 1996; 196(1):85-91, Roy A. et al., J Hematother. Stem Cell Res. 2001; 10(6):873-80, Dlu A. et al., Proc. Nati. Acad. Sci. USA Vol. 84, pp. 9140-9144, 1987, and EP0488470) and facilitates analysis and selection of secreted rabbit antibodies.

### Selection of a Single B Cell Which Comprises mRNA

Selection of a single B cell which comprises mRNA encoding polypeptides comprising a heavy and light chain variable region of an antibody according to the invention can be performed, preferably after co-cultivated with feeder cells, by analyzing the cell supernatant for secreted rabbit antibodies specifically binding to the IL-1RAcP antigen used for immunization. Analysis is preferably performed by ELISA. Immunoglobulin sequences can be then recovered from the selected single human B cell e.g. according to de Wildt R M, Hoet R M. Methods Mol. Biol. 2002; 178:121-31 and analyzed e.g. by RT PCR.

The production of an antibody according to the invention, expressed by a single B cell, can be performed by recombinant means.

Techniques and procedures described or referenced herein are for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

A Chinese hamster ovary tissue-derived CHO cell or cell line suitable in accordance with the present invention is any cell which is a cell line established from an ovary tissue of Chinese hamster (Cricetulus griseus). Examples include CHO cells described in documents such as Journal of Experimental Medicine, 108, 945 (1958); Proc. Nat Acad. Sci. USA, 60, 1275 (1968); Genetics, 55, 513 (1968); Chromosoma, 41, 129 (1973); Methods in Cell Science, 18, 115 (1996); Radiation Research, 148, 260 (1997); Proc. Nat Acad. Sci. USA, 77, 4216 (1980); Proc. Nat Acad. Sci., 60, 1275 (1968); Cell, 6, 121 (1975); Molecular Cell Genetics, Appendix I, II (pp. 883-900); and the like. In addition, CHO-K1 (ATCC CCL-61), DUXB1 1 (ATCC CCL-9096) and Pro-5 (ATCC CCL-1781) registered in ATCC (The American Type Culture Collection) as well as CHO-S (Life Technologies, Cat #1 1619) or sub-cell lines obtained by adapting the cell lines using various media can also be employed in the present invention.

In the following specific embodiments of the invention are listed:

The invention relates to a monoclonal antibody specifically binding to human IL-1RAcP.

It is preferably characterized in binding in addition to murine IL-1RAcP.

It is further preferably characterized in inhibiting IL-1RAcP induced NFkB activity.

It is further preferably characterized in inhibiting in addition murine IL-1RAcP induced murine NFkB activity.

It is further preferably characterized in inhibiting IL-1alpha, IL-1beta, IL-33, and/or IL-36 stimulated NFkB activity.

The antibody is characterized in inhibiting IL-1alpha stimulated NFkB activity.

The antibody is further characterized in inhibiting IL-1beta stimulated NFkB activity.

The antibody is also characterized in inhibiting IL-33 stimulated NFkB activity.

The antibody is further characterized in inhibiting IL-36 stimulated NFkB activity.

It is characterized in inhibiting NFkB activity stimulated by a complex selected from the group consisting of IL-1β/IL-1R1/IL-1RAcP, IL-1α/IL-1R1/IL-1RAcP IL-33/ST2/IL-1RAcP, and/or IL-36/Il-36R/IL-1RAcP.

The antibody is characterized in inhibiting in a concentration of 5 μg/ml (rabbit IgG isotype has a molecular weight of 150 KD) NFkB activity in 293T/17 cell lysates (293T/17 [HEK 293T/17] (ATCC® CRL-11268™)) stimulated with 0.5 μg/ml human IL-1alpha, IL-1beta, IL-33 and/or IL-36 (molecular weight see UniProtKB/Swiss-Prot), for 70% or more, preferably for 80% or more, preferably for 90% and more, and more preferably for 95% or more, related to the same assay without said antibody according to the invention.

Preferably the antibody is characterized in inhibiting in a concentration of 5 μg/ml NFkB activity in respective mouse cell line lysates stimulated with 0.5 μg/ml murine IL-1alpha, IL-1beta, IL-33 and/or IL-36 (molecular weight see UniProtKB/Swiss-Prot), for 70% or more, preferably for 80% or more, preferably for 90% and more, and more preferably for 95% or more, related to the same assay without said antibody according to the invention.

It inhibits IL-1alpha, IL-1beta, IL-33, and/or IL-36, respectively, stimulated luciferase activity in 293T/17 cells (293T/17-FR cells transfected with luciferase under control of NF-kB reporter gene). It is also characterized in exhibiting an ADCC reduced to at least 20% of the ADCC induced by the antibody according to the invention comprising a wild-type human IgG Fc region.

Preferably, it is characterized in exhibiting a reduced affinity to the human FcγRIIIA and/or FcγRIIA and/or FcγRI compared to an antibody according to the invention comprising the wildtype IgG Fc region, and wherein the ADCC induced by said antibody according to the invention is reduced to at least 20% of the ADCC induced by the antibody according to the invention comprising a wild-type human IgG Fc region.

The antibody is characterized in comprising at least amino acid substitutions at L234A and L235A of the human IgG1 Fc region or S228P and L235E of the human IgG4 Fc region.

The antibody is further characterized in that the heavy chain variable (VH) region is at least 90% identical to a VH region selected from the group consisting of VH regions of SEQ ID NO:1 to 77.

The antibody is preferably characterized in that the light chain variable (VL) region is at least 90% identical to a VL region selected from the group consisting of VL regions of SEQ ID NO:78 to 154.

The antibody is also preferably characterized in that its VH region is at least 90% identical to a VH region of SEQ ID NO:1+n and its VL region is at least 90% identical to a VL region of SEQ ID NO:78+n, wherein n is a number selected from the group consisting of 0 to 76.

Preferred is an antibody according to the invention characterized in that the antibody comprises a VH region comprising a heavy chain CDRH1 sequence selected from SEQ ID NO: 214, 216, 219, 220, 221, 228, 156, 159, 183, 164, 163, 161, 157, 155, 174, 166, 173, 177, 158, a CDRH2 sequence selected from the group of SEQ ID NO: 291, 293, 296, 297, 298, 305, 233, 236, 260, 241, 240, 238, 234, 232, 251, 243, 250, 254, 235, and a CDRH3 sequence selected from the group of SEQ ID NO: 368, 370, 373, 374, 375, 382, 310, 313, 337, 318, 317, 315, 311, 309, 328, 320, 327, 331, 312, respectively.

Also preferred is an antibody characterized in that the antibody comprises a VL region comprising a light chain CDRL1 sequence selected from the group of SEQ ID NO: 445, 447, 450, 451, 452, 459, 387, 390, 414, 395, 394, 392, 388, 386, 405, 397, 404, 408, 389, a CDRL2 sequence selected from the group of SEQ ID NO: 522, 524, 527, 528, 529, 536, 464, 467, 491, 472, 471, 469, 465, 463, 482, 474, 481, 485, 466, and a CDRL3 sequence selected from the group of SEQ ID NO: 599, 601, 604, 605, 606, 613, 541, 544, 568, 549, 548, 546, 542, 540, 559, 551, 558, 562, 543, respectively.

In a further embodiment the antibody is characterized in that said VH region is selected from the group consisting of VH regions of SEQ ID NO:1 to 77.

It is also preferred that the antibody is characterized in that said VL region is selected from the group consisting of VL regions of SEQ ID NO: 78 to 154.

Further preferred is an antibody characterized in that its VH region is selected from the group consisting of VH regions of SEQ ID NO: 60, 62, 65, 66, 67, 74, 2, 5, 29, 10, 9, 7, 3, 1, 20, 12, 19, 23, 4.

Preferably, the heavy chain variable region (VH) sequence is SEQ ID NO: 60, alternatively SEQ ID NO:62, or SEQ ID NO:65, SEQ ID NO:66, SEQ ID NO:67, SEQ ID NO:74, SEQ ID NO:2, SEQ ID NO:5, SEQ ID NO:29, SEQ ID NO:10, SEQ ID NO:9, SEQ ID NO:7, SEQ ID NO:3, SEQ ID NO:1, SEQ ID NO:20, SEQ ID NO:12, SEQ ID NO:19, SEQ ID NO:23, or alternatively SEQ ID NO:4.

Further preferred is an antibody characterized in that its VL region is selected from the group consisting of VL regions of SEQ ID NO: 137, 139, 142, 143, 144, 151, 79, 82, 106, 87, 86, 84, 80, 78, 97, 89, 96, 100, 81.

Preferably, the light chain variable region (VL) sequence is SEQ ID NO: 137, alternatively SEQ ID NO: 139, or SEQ ID NO: 142, SEQ ID NO: 143, SEQ ID NO: 144, SEQ ID NO: 151, SEQ ID NO: 79, SEQ ID NO: 82, SEQ ID NO: 106, SEQ ID NO: 87, SEQ ID NO: 86, SEQ ID NO: 84, SEQ ID NO: 80, SEQ ID NO: 78, SEQ ID NO: 97, SEQ ID NO: 89, SEQ ID NO: 96, SEQ ID NO: 100, or alternatively SEQ ID NO: 81.

Most preferred is an antibody characterized in that its VH region is selected from the group consisting of VH regions of SEQ ID NO: 60, 62, 65, 66, 67, 74, 2, 5, 29, 10, 9, 7, 3, 1, 20, 12, 19, 23, 4, and its VL region is selected from the group consisting of VL regions of SEQ ID NO: 137, 139, 142, 143, 144, 151, 79, 82, 106, 87, 86, 84, 80, 78, 97, 89, 96, 100, 81.

In one embodiment, the antibody according to the invention comprises SEQ ID NO.: 137 and 60, or SEQ ID NO.: 139 and 62. An antibody according to the invention may also comprise SEQ ID NO.: 142 and 65, or SEQ ID NO.: 143 and 66, or SEQ ID NO.: 144 and 67, SEQ ID NO.: 151 and 74, or SEQ ID NO.: 79 and 2, or SEQ ID NO.: 82 and 5, or SEQ ID NO.: 106 and 29, or SEQ ID NO.: 87 and 10, or SEQ ID NO.: 86 and 9, or SEQ ID NO.: 84 and 7, or SEQ ID NO.: 80 and 3, or SEQ ID NO.: 78 and 1. Alternatively, an antibody according to the invention comprises SEQ ID NO.: 97 and 20, or SEQ ID NO.: 89 and 12, or SEQ ID NO.: 96 and 19, or SEQ ID NO.: 100 and 23, or SEQ ID NO.: 81 and 4.

Particularly preferred is an antibody according to the invention comprising SEQ ID NO.: 79 and 2, or SEQ ID NO.: 81 and 4, or SEQ ID NO.: 139 and 62, or SEQ ID NO.: 80 and 3, or SEQ ID NO.: 78 and 1.

The antibody is preferably characterized in that its VH region is selected from the group consisting of VH regions of SEQ ID NO: 1+n and its VL region is selected from the group consisting of VL regions of SEQ ID NO: 78+n, wherein n is a number selected from the group consisting of 0 to 76.

In a further embodiment the antibody is characterized in that the antibody comprises a VH region selected from the group of VH regions comprising a CDR1H region of SEQ ID NO: 155+n, a CDR2H region of SEQ ID NO: 232+n and aCDR3H region of SEQ ID NO: 309+n, wherein n is a number selected from the group consisting of 0 to 76.

The antibody is preferably characterized in that the antibody comprises a VL region selected from the group of VL regions comprising a CDR1L region of SEQ ID NO: 386+n, a CDR2L region of SEQ ID NO: 463+n and aCDR3L region of SEQ ID NO: 540+n, wherein n is a number selected from the group consisting of 0 to 76.

The antibody is preferably characterized in that the antibody comprises a VH region selected from the group of VH regions comprising a CDR1H region of SEQ ID NO: 155+n, a CDR2H region of SEQ ID NO: 232+n and aCDR3H region of SEQ ID NO: 309+n, and in that the antibody comprises a VL region selected from the group of VL regions comprising a a CDR1L region of SEQ ID NO: 386+n, a CDR2L region of SEQ ID NO: 463+n and aCDR3L region of SEQ ID NO: 540+n, wherein n is a number selected from the group consisting of 0 to 76.

The antibody may be characterized in comprising a VH region and a VL region comprising the respective CDR1, CDR2 and CDR3 regions of an antibody selected from the group consisting of antibodies listed in FIG. 2.

Preferably the antibody is characterized in inhibiting IL-1RAcP induced NFkB activity, binding to the same epitope as an antibody selected from the group of antibodies P013.S.01.B.B03, P013.S.01.B.A05, P013.S.01.B.C04, P013.S.01.B.H01, P013.S.01.B.D03, P013.S.01.B.E02, P013.S.02.B.A04, P013.S.02.B.A05, P013.S.02.B.A02, P013.S.02.B.D03, P013.S.02.B.H01, P013.S.02.B.F01, P013.S.02.B.B04, P013.S.02.B.C02, P013.S.02.B.B05, P013.S.02.B.A03, P013.S.02.B.H03, and P013.S.02.B.G05.

In one embodiment the antibody is characterized in being a rabbit/human chimeric or humanized antibody.

The invention also relates to a method for the production of a monoclonal rabbit antibody against human IL-1RAcP characterized in inhibiting IL1beta stimulated NFkB activity according to the invention, characterized in
i) that after immunizing said rabbit with IL-1RAcP, a number of antibody producing single cells derived from said rabbit are isolated,
ii) binding to IL-1RAcP is measured separately for the supernatants of said single cells,
iii) a single cell is selected if its supernatant shows binding to human IL-1RAcP and murine, and inhibits NFkB activity stimulated by IL-1alpha, IL-1beta, IL-33 and/or IL-36,
iv) an antibody with the properties of iii) is isolated from said selected cell.

Preferably the method is characterized in that the rabbit antibody producing single cell is a single B rabbit hybridoma cell.

The method is also characterized in that after immunizing said rabbit with said antigen, a single antibody producing cell is isolated from said animal or a rabbit hybridoma cell derived from said rabbit is isolated, for which binding to human IL-1RAcP, and inhibition of NFkB activity stimulated by IL-1alpha, IL-1beta, IL-33 and/or IL-36 is found.

The invention relates to the use of the antibody for the manufacture of a pharmaceutical composition.

It relates to a supernatant of a rabbit antibody producing single cell, characterized in binding to human IL-1RAcP, and inhibition of NFkB activity stimulated by IL-1alpha, IL-1beta, IL-33 and/or IL-36.

The invention relates to a method of treating an IL-1 mediated disease in a patient, comprising administering to a patient a pharmaceutically effective amount of the antibody.

The invention relates to a pharmaceutical composition comprising a pharmaceutically acceptable carrier and a therapeutically effective amount of the antibody according to any one of embodiments.

It also relates to a method of treating an IL-1 mediated disease in a patient, comprising administering to a patient the present pharmaceutical composition.

### EXAMPLES

### Example 1: Compounds

| | Host/ | | Com- | |
|---|---|---|---|---|
| MAB ID | species | Substance | pany | Cat. No. |
| P013_01 | | rhIL-1RAcP/Fc Chimera | R&D | 676-CP |
| P013_02 | rabbit | IL1RAcP purified | Abnova | H00003556- |
| | | MaxPab rabbit polyclonal | | D01P |
| | | Ab (D01P) against | | |
| | | TARDBP (NP_031401.1) | | |
| P013_03 | human | rhIL-1RAcP/Fc Chimera | | |
| P013_04 | murine | mIL1RAcP-Fc | | |
| P013_05 | human | recombinant human IL-1ß | R&D | 201-LB-005 |
| P013_06 | goat | Human IL-1 RAcP/ | R&D | AF676 |
| | | IL-1 R3 affinity | | |
| | | purified polyclonal antibody | | |
| P013_07 | human | hIL1RAcP | | C477 |

### Example 2: Immunization of Rabbits

Rabbits were immunized with hu-IL-1RAcP-Fc repeatedly. Blood of these animals was collected and B lymphocytes thereof were isolated. Single B-cells were sorted into wells of microtiter plates and propagated. Supernatants conditioned by these B-cells were analyzed in hu-IL-1RAcP ELISA. 409 monoclonal antibodies (=4.7% of all tested supernatants) were identified to bind to hu-IL-1RAcP. 23 monoclonal antibodies were found to bind also to murine IL-1RAcP and inhibit IL1beta induced human or murine NF-κB activity.

### a) Immunization of Rabbits (Scheme 1)

Recombinant human Fc-chimera proteins fused human IL-1RAcP (IL-1RAcP-Fc) was used as immunogen. Two different immunization schemes, scheme 1 and scheme 2, were explored. For the immunization according to scheme 1, three New Zealand White (NZW) rabbits were immunized by injecting 1ml of immunogen in each of the animals at day 0, 7, 14, 28, 42, and 56. Proteins were diluted in PBS, pooled in equimolar amounts and mixed 1:1 (v/v) with complete Freund's adjuvant (CFA) before use. A final concentration of 400 μg of immunogen was used per animal for the 1st immunization and for the 2nd, 3rd, 4th, 5th and 6th immunization 200 μg of immunogen and per animal was used. Blood samples were collected in tubes, coated with EDTA, five, six and seven days post-immunization after the 3rd, 4th, 5th and 6th immunization. Anti IL-1RAcP antibodies according to the invention were isolated from the blood sample taken after the third immunization. Antibodies according to the invention were isolated from blood samples taken after the 3rd, 4th, 5th and 6th immunization.

### b) Immunization of Rabbits (Scheme 2)

For the immunization according to scheme 2, each of the six NZW rabbits were immunized subcutaneously with 1ml of immunogen at day 0, 7, 14, 28, 42, 56, 70 and 84. For the first injection, proteins were diluted in PBS, pooled in equimolar amounts and mixed 1:1 (v/v) with CFA before use. A final concentration of 200 μg of Immunogen per animal was used for the 1st immunization. For the 2nd, 3rd, 4th, 5th and 6th immunization, proteins were diluted in PBS, pooled in equimolar amounts and mixed 1:1 (v/v) with incomplete Freund's adjuvant (IFA) before use. 100 μg of Immunogen was used per animal. Blood samples were collected in tubes, coated with EDTA, five six and seven days post-immunization after the 3rd, 4th, 5th and 6th Immunization at intervals of 2 weeks.

### Example 3

### Immunogen Coating/Cell Preparation

The fusion-protein used for immunization was coated onto a surface of a cell-culture 6-well plate with a concentration of 8 μg in PBS/10cm2 and incubated. Alternatively, plates were seeded with a cell line BT-474 (DSMZ ACC 64) on their cell surface. One day before use cells were seeded in DMEM+5% FCS at a density leading to about 90% confluence after 24 h.

### Isolation of Peripheral Blood Mononuclear Cells From Rabbits

PBMCs were isolated from whole blood of immunized rabbits. The blood was diluted 1:1 with PBS and layered on Lympholyte® according to the manufacturer's instructions (Cedarlane, CL5120). Peripheral blood mononuclear cells (PBMC) were separated from erythrocytes by density gradient centrifugation (800×g, 20 min, RT). Cells were removed from the interface, washed twice with PBS (800×g, 10 min) and suspended in RPMI 1640 based cell culture medium.

### Monocyte Depletion

PBMCs were incubated in cell culture medium on plastic. Unbound lymphocytes were collected after incubation time.

### Enrichment of Antigen Specific Cells

Antigen specific lymphocytes were enriched on immunogen coated plates or directly on BT-474 cells. Lymphocytes were washed twice with PBS to remove unspecific cells and subsequently incubated with 750 μl Trypsin per 10cm2 culture surface for 7-10 min. Detached cells were collected in cell culture medium for further steps.

### Single-Cell Sorting of Immunoglobulin G-Secreting Lymphocytes

PBMCs/lymphocytes were stained with a FITC (Fluorescein Isothiocyanate Isomer 1) conjugated goat anti-rabbit IgG antibody, Abd Serotec, STAR121F). A flow cytometric analysis and single-cell sorting was performed with a FACS cytometer. Single positive lymphocytes were sorted directly to 200 μl cell culture medium covering 3.0×106 irradiated EL-4 B5 feeder cells. The cell culture medium described above was supplemented with 5% activated T-cell macrophage supernatant from rabbits (MicroCoat). Co-cultivation medium was supplemented with 2×10−06 g/ml SAC (Staphylococcus aureus Cowan) solution. After co-cultivation of B-cells and feeder cells for 7 days supernatants were transferred for antibody detection and cells were harvested in 100 μl RNA isolation buffer (Qiagen, RLT).

### Screening for Immunoglobulin's Via Enzyme-Linked Immunosorbent Assay

Secreted rabbit antibodies were detected by analyzing the supernatant via a biotinylated capturing antibody (anti-rabbit IgG antibody produced in goat) with a final concentration of 1 μg/ml PBS+0.5% BSA+0.05% Tween® 20, coated on streptavidin microtiter plates and a horse radish peroxidase coupled anti-rabbit IgG detection antibody with a final concentration of 1:7500. Washing steps were performed by using PBS+0.1% Tween® 20. 3,3′,5,5′-Tetramethylbenzidine (TMB) was used as substrate and HCl to stop the enzymatic reaction.

### Determination of IL-1RACP Specific Antibodies in B-Cell Supernatants

Microtiter plates were coated IL-1RACP and/or IL12Rβ1 protein (recombinant Fc chimeric conjugates of human IL-1RACP or IL12Rβ1). After a blocking process, specific antibodies from B-cell supernatants bind to the targets and are then detected by a POD-labeled anti-rabbit IgG antibody. The IL12Rβ1 binding was used as a counter screen. IL-1RACP protein was tagged with a linker, huFc and His like the IL12Rβ1 protein. Antibodies which bind to the tag were positive in both assays, whereas antigen specific antibodies just bound to IL-1RACP and not to IL12Rβ1.

12.5 μL 0.5 μg/mL IL-1RACP protein in PBS was transferred to a microtiter plate, incubated and washed 3× with Wash Buffer. 90 μL Block Buffer was added to each well, incubated and washed. 12.5 μl Standard Antibody (rabbit mAb against IL-1RAcP, anti IL12Rbeta1 antibody: IL-12Rbeta1 antibody; GeneTex; Cat. No. GTX103917) or sample diluted in ELISA buffer was added, incubated and washed. 12.5 μl 1:5000 POD-Antibody (Anti-rabbit IgG, peroxidase-linked species-specific Fab2 fragment (from donkey) (ECL); assay dilution: 1:5000) in Elisa Buffer was added, incubated and washed. 15 μl TMB was added and 15 μl HCl was added after sufficient development. Absorbance (Optical Density O.D.) was read at 450 nm/620 nm. Results are shown in FIG. 1.
ELISA Buffer: PBS, 0.5% BSA, 0.05% Tween® 20
Wash Buffer: PBS, 0.1% Tween® 20
Block Buffer: PBS, 2% BSA, 0.05% Tween® 20

### Example 4: Antibody Binding to Human IL-1RAcP

### Assay Principle

NUNC Maxisorp® 384well microtiter plates are coated with P013_03. After a blocking process, specific antibodies from B-cell supernatants bind to the antigen human (P013-03) or murine IL-1RACP (P013-04) and are then detected by a POD-labeled antibody. Samples are tested 1:2 diluted.

### Materials

Plates: 384 well NUNC Maxisorp® plates; Cat. No. 464718
Proteins: P013-03 (Conc. 1.5 mg/ml; Assay Conc. 0.5 μg/ml) human
   P013-04 (Conc. 1.3 mg/ml; Assay Conc. 0.5 μg/ml) murine
Standard Ab: P013-02 (Conc. 1 mg/ml; Start Assay Conc. 2 μg/ml)
Detection Ab: Anti-rabbit IgG, peroxidase-linked species-specific whole antibody (from donkey) (ECL); GE; Cat. No. NA9340; assay dilution: 1:5000
PBS: Buffers in a Box, Premixed PBS Buffer, 10×; Roche Applied Sciences; Cat. No. 11666789001
BSA: Bovine Serum Albumin Fraction V from bovine serum; Roche Applied Sciences; Cat. No. 10735086001
Tween® 20: Tween® 20; Carl Roth; Cat. No. 9127.2
TMB: TMB Solution; Life Technologies; Cat. No. SB02
HCl: 1M Titripur® Hydrochloric Acid; Merck; Cat. No. 1090571000
ELISA Buffer: PBS, 0.5% BSA, 0.05% Tween®
Wash Buffer: PBS, 0.1% Tween®
Block Buffer: PBS, 2% BSA, 0.05% Tween®
Samples: 1:2 dilution in Elisa Buffer

### Procedure

1. Add 12.5 μL P013-03 (0.5 μg/ml) in PBS to a 384 well NUNC Maxisorp® plate and incubate for 1 h at RT.
2. Wash 3× with 90 μl Wash Buffer.
3. Add 90 μL Blocking buffer to each well and incubate for 1 h at RT.
4. Wash 3× with Wash Buffer.
5. Add 12.5 μL Standard Antibody in 1:2 dilutions or sample 1:2 diluted in Elisa Buffer and incubate for 1 h at RT.
6. Wash 3× with Wash Buffer.
7. Add 12.5 μL 1:5000 POD-Antibody in Elisa Buffer and incubate for 1 h at RT.
8. Wash 6× with Wash Buffer.
9. Add 15 μL TMB.
10. Add 15 μL HCl after sufficient development.
11. Read absorbance at 450 nm/620 nm.

### Example 5: Antibody Binding to Murine IL-1RAcP

### Assay Principle

NUNC Maxisorp® 384well microtiter plates are coated with P013_04. After a blocking process, specific antibodies from B-cell supernatants bind to the antigen and are then detected by a POD-labeled antibody. Samples are tested 1:2 diluted.

### Materials

Plates: 384 well NUNC Maxisorp® plates; Cat. No. 464718
Proteins: P013-04 (Conc. 1.3 mg/ml; Assay Conc. 0.5 μg/ml)
Standard Ab: P013-02 (Conc. 1 mg/ml; Start Assay Conc. 2 μg/ml)
Detection Ab: Anti-rabbit IgG, peroxidase-linked species-specific whole antibody (from donkey) (ECL); GE; Cat. No. NA9340; assay dilution: 1:5000
PBS: Buffers in a Box, Premixed PBS Buffer, 10×; Roche Applied Sciences; Cat. No. 11666789001
BSA: Bovine Serum Albumin Fraction V from bovine serum; Roche Applied Sciences; Cat. No. 10735086001
Tween 20: Tween® 20; Carl Roth; Cat. No. 9127.2
TMB: TMB Solution; Life Technologies; Cat. No. SB02
HCl: 1M Titripur® Hydrochloric Acid; Merck; Cat. No. 1090571000
ELISA Buffer: PBS, 0.5% BSA, 0.05% Tween®
Wash Buffer: PBS, 0.1% Tween®
Block Buffer: PBS, 2% BSA, 0.05% Tween®
Samples: 1:2 dilution in Elisa Buffer

### Procedure:

1. Add 12.5 μL P013-04 (0.5 μg/ml) in PBS to a 384well NUNC Maxisorp® plate and incubate for 1 h at RT.
2. Wash 3× with 90 μl Wash Buffer.
3. Add 90 μL Blocking buffer to each well and incubate for 1 h at RT.
4. Wash 3× with Wash Buffer.
5. Add 12.5 μL Standard Antibody in 1:2 dilutions or sample 1:2 diluted in Elisa Buffer and incubate for 1 h at RT.
6. Wash 3× with Wash Buffer.
7. Add 12.5 μL 1:5000 POD-Antibody in Elisa Buffer and incubate for 1 h at RT.
8. Wash 6× with Wash Buffer.
9. Add 15 μL TMB.
10. Add 15 μL HCl after sufficient development.
11. Read absorbance at 450 nm/620 nm.

### Example 6: EC50 Determination in ELISA

The binding of an antibody according to the invention to human IL-1RAcP was analyzed in ELISA: EC50 values were calculated according to the state of the art.

### Example 7: NF-κB Neutralizing Activity of Antibodies Against IL-1RAcP in a Luciferase-Based Genetic Reporter Assay

### Assay Principle

293T/17-FR cells, which express a NF-kB-RE firefly luciferase reporter, are seeded into Poly-D-lysine-Cell culture plates. After stimulation of P013 the 293T/17-FR lysate is tested for activated NF-kB using the Steady-Glo Luciferase Assay Kit. Supernatants with functional antibodies bind to P013 and inhibit the NF-kB activation, which is shown in low signal. Samples are tested 1:2 diluted in P013 solution.

### Materials

Plates: Cell plate: 384well PDL Costar Cell Culture plate; Cat. No. 3844
Assay plate: 384well Lumitrac® white-plate; Corning; Cat. No. 3572
Cells: 293T/17-FR; assay conc. 250.000 cells/ml
Proteins: P013_05 (Conc. 0.03 mg/ml; Assay Conc. 115 pg/ml; Working Conc. 230 pg/ml) IL-1alpha, IL-33 and IL-36
Standard Ab: P013_06 (Conc. 0.2 mg/ml; Start Working Conc. 6 μg/ml)
Kit: Steady-Glo Luciferase Assay System; Promega; Cat. No. E2510
Cell-Medium: DMEM Medium; PAN Biotech; Cat. No. P04-04510
FCS: Fetal Bovine Serum, HyClone; Thermo; Cat. No. St30070.03
293T/17-FR Medium: DMEM Medium, 10% FCS, (+20 μg/ml Hygromycin-B, just for cultivation) Conditioned B-cell Medium (MAB Discovery)
Samples: 1:2 dilution with P013_05 in DMEM-Medium+10% FCS

### Procedure

1. Split confluent 293T/17-FR cells every Monday (seed out: 5×106 cells/T175 flask) and Friday (seed out: 3×106 cells/T175 flask) using trypsin/EDTA (incubate just for 30 sec at RT).
2. Seed cells (0.25×106 cells/ml) in 25 μl DMEM+10% FCS to a 384-well PDL-plate (Corning cat #3844) and incubate over night at 37° C. and 5% CO2.
3. Aspirate media and add 12.5 μl Sample or P013_06 in 1:3 dilution in Conditioned Medium or just Conditioned Medium and incubate for 30 min at 37° C. and 5% CO2 (program: 3 Aspiration and Sample transfer)
4. Add 12.5 μl P013_05 in DMEM+10% FCS and incubate for 5 hours at 37° C. and 5% CO2 (program: 4_Add P013_05).
5. Equilibrate cultured cells to RT for 10 min.
6. Add 25 μl Steady-Glo® Reagent and mix several times with pipette (program: 6_Steady Glo®)
7. Wait 5 minutes before transfer 45 μl supernatant to a 384-well Lumitrac® white plate (Corning Cat #3572) (program: 7_Transfer 45 ul)
8. Measure luminescence in Tecan Reader (Tecan Group Mannedorf, CH): Integration Time: 0.5 sec

### Example 8: IL-1α Neutralization Assay

### Materials

Cells: HEK-293T cells stably expressing firefly luciferase NF-kB reporter and Renilla Luciferase (for normalization control). IL1RAcP and the IL1R1 are endogenously expressed.
Media: DMEM (ATCC Cat #30-2002)+10% heat inactivated FBS
Reagents IL-1α-R&D #200-LA; 10 μg/ml PBS+0.1% BSA
   Anti-IL1RAcP Positive Control Antibody—R&D #AF676; 200 ug/ml
   MAB Discovery Antibodies—Plate 1 antibodies at 750 ug/ml.
      Plate 2 antibodies at 250 ug/ml
   Luciferase Assay System—Promega #E1500

### Procedure

1. Plate cells at 50,000/well into 96 well plate in 100 ul DMEM+10% FBS. Incubate overnight 37° C., 5% CO₂
2. Prepare 4 fold dilution of antibodies at 2× final concentration in DMEM+10% FBS
3. Aspirate media off cells and add antibodies at 2× final concentration in 60 ul DMEM+10% FBS. Incubate cells 30 m at 37° C. 5% CO₂
4. Add IL-1α to 175 pg/ml final concentration in 60 ul complete media. (175 pg/ml is the EC50.) Incubate 4 h at 37° C. 5% CO₂
5. Wash cells with 150 ul PBS
6. Lyse cells in 50 ul 1× cell culture lysis reagent (from Luciferase Assay System) for 15 m on shaker at ambient temperature.
7. Pipet up and down and transfer 20 ul lysate to lumitrac-200 plate. Add 100 ul luciferase assay reagent and read luminescence using Wallac Victor2 with liquid injector or other suitable luminometer.

### Example 9: IL-1β Neutralization Assay

### Materials

Cells: HEK-293T cells stably expressing firefly luciferase NF-kB reporter and Renilla Luciferase (for normalization control). IL1RAcP and the IL1R1 are endogenously expressed.
Media: DMEM (ATCC Cat #30-2002)+10% heat inactivated FBS
Reagents: IL-1β-R&D #201-LB; 25 ug/ml PBS+0.1% BSA
   Anti-IL1RAcP Positive Control Antibody—R&D #AF676; 200 ug/ml
   Anti-IL1RAcP Rabbit pAb Positive Control Antibody—ONCO Lot AP14/200 ug/ml PBS
   Normal Rabbit IgG—JL #011-000-003; 200 ug/ml PBS
   MAB Discovery Antibodies—Plate 1 antibodies at 750 ug/ml.
      Plate 2 antibodies at 250 ug/ml
   Luciferase Assay System-Promega #E1500

### Procedure

1. Plate cells at 50,000/well into 96 well plate in 100 ul DMEM+10% FBS. Incubate overnight 37° 5% CO₂
2. Prepare 4 fold dilution of antibodies at 2× final concentration in DMEM+10% FBS
3. Aspirate media off cells and add antibodies at 2× final concentration in 60 ul DMEM+10% FBS. Incubate cells 30 m at 37° 5% CO₂
4. Add IL-1β to 175 pg/ml final concentration in 60 ul complete media. 175 pg/ml is the EC₅₀. Incubate 4 h at 37° 5% CO₂
5. Wash cells with 150 ul PBS
6. Lyse cells in 50 ul 1× cell culture lysis reagent (from Luciferase Assay System) for 15 m on shaker at ambient temperature.
7. Pipet up and down and transfer 20 ul lysate to lumitrac-200 plate. Add 100 ul luciferase assay reagent and read luminescence using Wallac Victor2 with liquid injector or other suitable luminometer.

### Example 10: IL-33 Neutralization Assay

### Materials

Cells: HEK-293T cells transiently transfected with firefly luciferase NF-kB reporter, renilla luciferase (for normalization control), and IL-33R driven by the CMV promoter. IL1RAcP is endogenously expressed.
Media: DMEM (ATCC Cat #30-2002)+10% heat inactivated FBS
Reagents: IL-33—R&D #3625-IL; 10 ug/ml PBS+0.1% BSA
   Anti-IL1RAcP Positive Control Antibody—R&D #AF676; 200 ug/ml
   MAB Discovery Antibodies—Plate 1 antibodies at 750 ug/ml.
      Plate 2 antibodies at 250 ug/ml
   Luciferase Assay System-Promega #E1500

### Procedure

1. Transfect cells with luciferase reporters and IL-33R at 25,000 cells/well approximately 24 h before assay.
2. Prepare 4 fold dilution of antibodies at 2× final concentration in DMEM+10% FBS
3. Aspirate media off cells and add antibodies at 2× final concentration in 60 ul DMEM+10% FBS. Incubate cells 30 m at 37° 5% CO₂
4. Add IL-33 to 250 pg/ml final concentration in 60 ul complete media. Incubate 4 h at 37° 5% CO₂
5. Wash cells with 150 ul PBS
6. Lyse cells in 50 ul 1× cell culture lysis reagent (from Luciferase Assay System) for 15 m on shaker at ambient temperature.
7. Pipet up and down and transfer 20 ul lysate to lumitrac-200 plate. Add 100 ul luciferase assay reagent and read luminescence using Wallac Victor2 with liquid injector or other suitable luminometer.

### Example 11: IL-36β (IL1F8) Neutralization Assay

### Materials

Cells: HEK-293T cells stably transfected with firefly luciferase NF-kB reporter, renilla luciferase (for normalization control), and IL-36R driven by the CMV promoter. IL1RAcP is endogenously expressed.
Media: DMEM (ATCC Cat #30-2002)+10% heat inactivated FBS
Reagents: IL-36β-R&D #6834-IL; 100 ug/ml PBS+0.1% BSA
   Anti-IL1RAcP Positive Control Antibody—R&D #AF676; 200 ug/ml
   MAB Discovery Antibodies—Plate 1 antibodies at 750 ug/ml.
      Plate 2 antibodies at 250 ug/ml
   Luciferase Assay System—Promega #E1500

### Procedure

1. Plate cells at 50,000/well into 96 well plate in 100 ul DMEM+10% FBS. Incubate overnight 37° 5% CO₂
2. Prepare 4-fold dilution of antibodies at 2× final concentration in DMEM+10% FBS
3. Aspirate media off cells and add antibodies at 2× final concentration in 60 ul DMEM+10% FBS. Incubate cells 30 m at 37° 5% CO₂
4. Add IL-36β to 15 ng/ml final concentration in 60 ul complete media. (15 ng/ml is the EC₅₀.) Incubate 4 h at 37° 5% CO₂
5. Wash cells with 150 ul PBS
6. Lyse cells in 50 ul 1× cell culture lysis reagent (from Luciferase Assay System) for 15 m on shaker at ambient temperature.
7. Pipet up and down and transfer 20 ul lysate to lumitrac-200 plate. Add 100 ul luciferase assay reagent and read luminescence using Wallac Victor2 with liquid injector or other suitable luminometer.

### FIGURE LEGENDS

FIG. 1: Antibody binding to human and murine IL-1RAcP

Results of experiments described in examples 3-6.

FIG. 2: Sequences (amino acids in one letter code)

D, A, S, K, L, A, S means DASKLAS. The same holds true for all other sequences of FIG. 2.

CDRH1: SEQ ID NO: 155-231

CDRH2: SEQ ID NO: 232-308

CDRH3: SEQ ID NO: 309-385

CDRL1: SEQ ID NO: 386-462

CDRL2: SEQ ID NO: 463-539

CDRL3: SEQ ID NO: 540-616

FIG. 3: Inhibition of ligand induced signaling

Summary table of results of signaling inhibition for the most promising 18 antibodies is shown. Experimental procedures are detailed in examples 8-11.

FIG. 4: Inhibition of ligand induced signaling by selected antibodies

Exemplary graphs of results from experiments described in examples 8-11. Shown is the percentage of nFkB stimulation due to signaling of different ligands, as cited in the title of each figure. Different colors correspond to different antibodies. Only a selection of tested antibodies is shown.

FIG. 5: NF-κB neutralizing activity of selected antibodies against IL-1RAcP

Results of experiments described in Example 7. Shown is the NF-κB neutralizing activity of antibodies against IL-1RAcP in a luciferase-based genetic reporter assay.

FIG. 6: Inhibition of ligand induced signaling of antibodies with preferred sequences

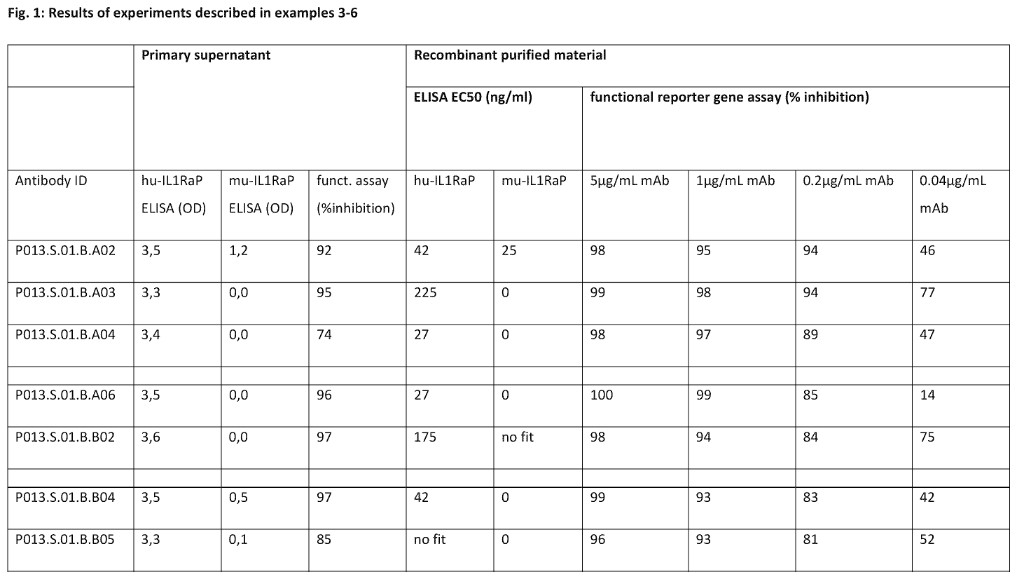

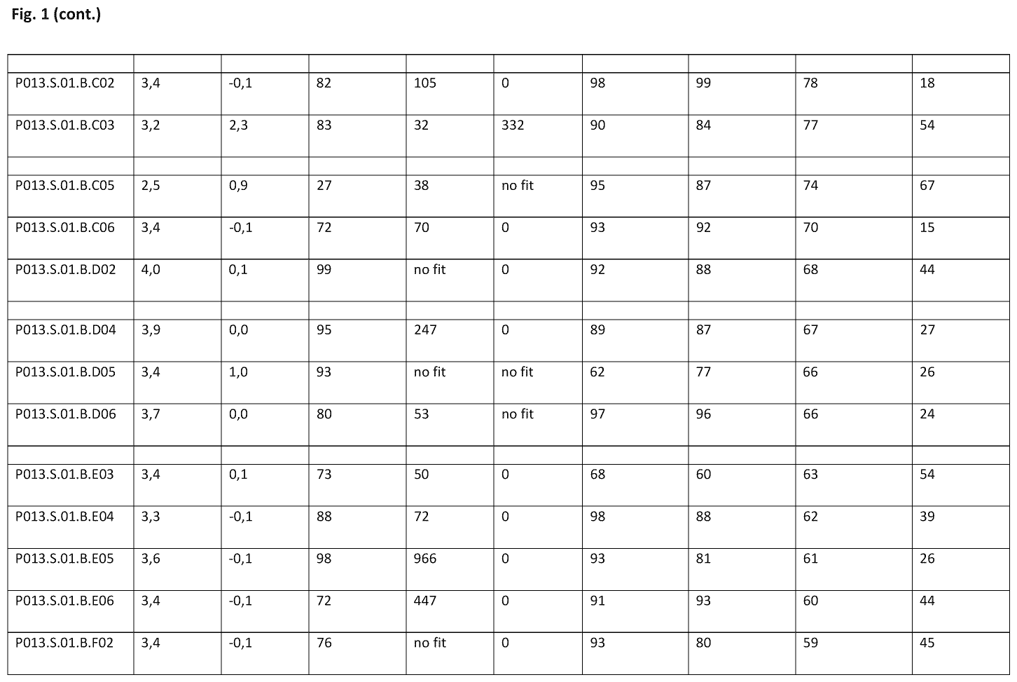

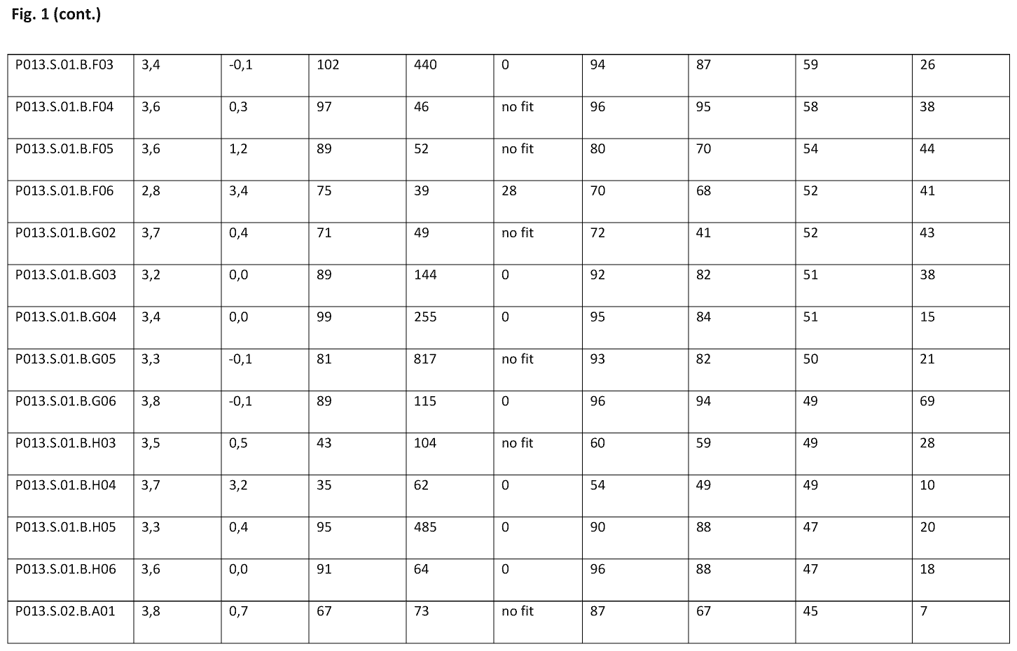

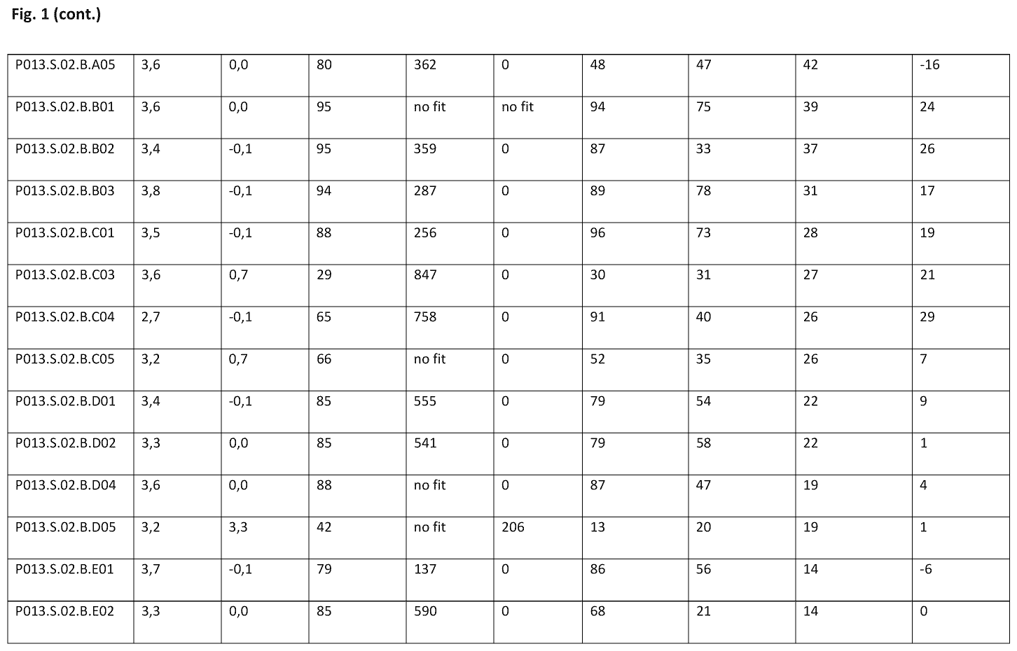

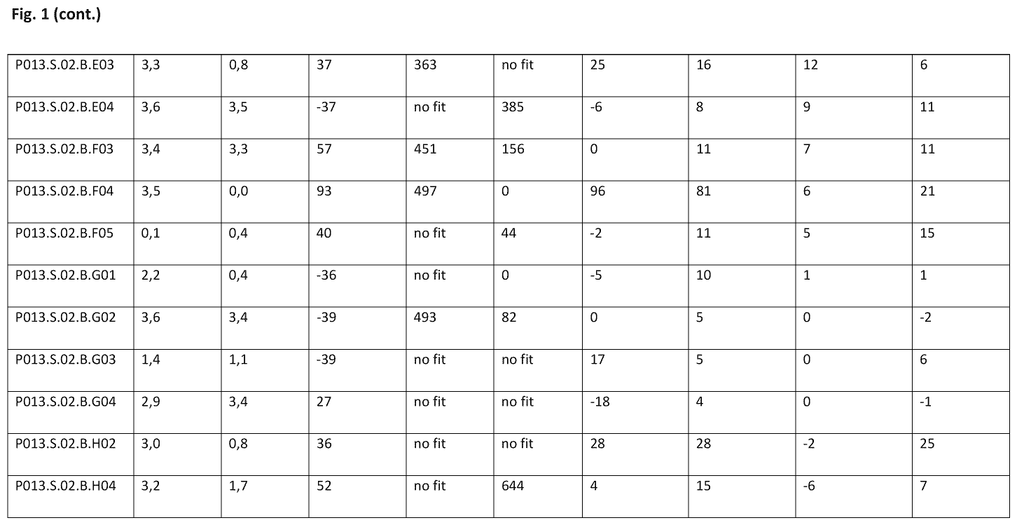

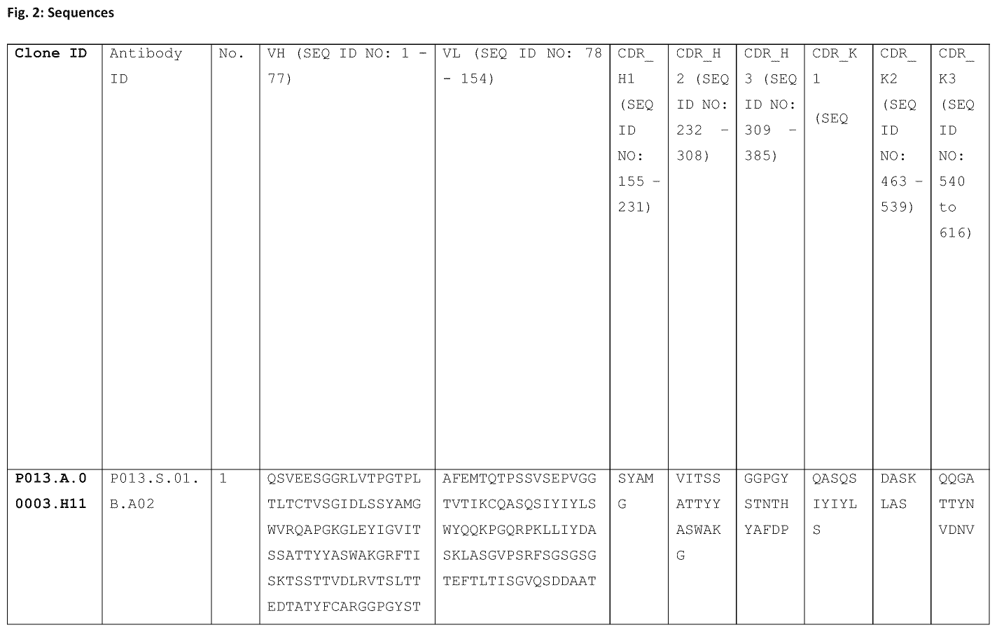

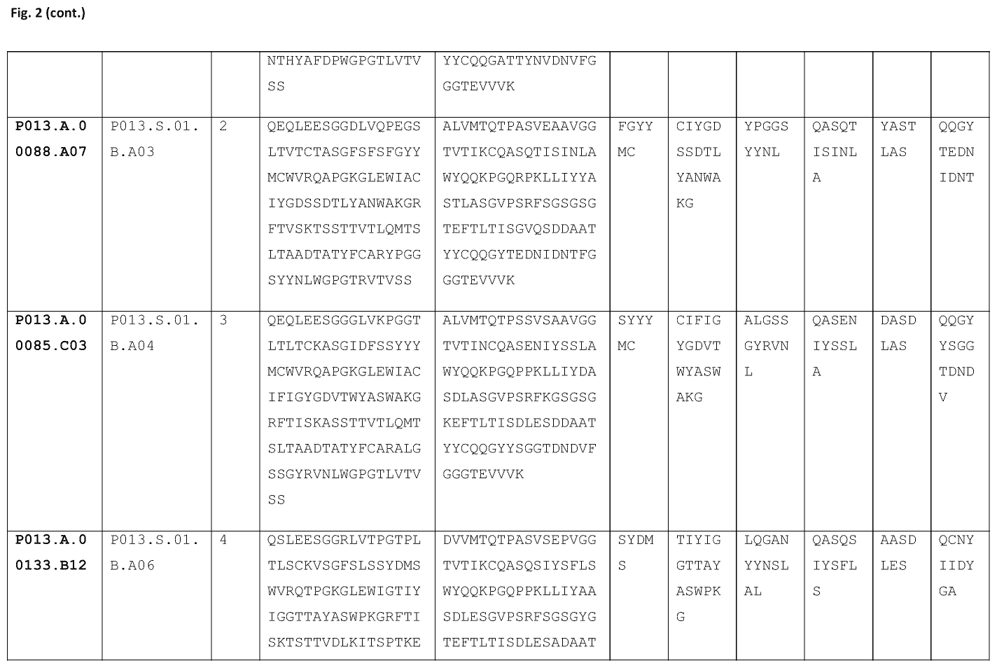

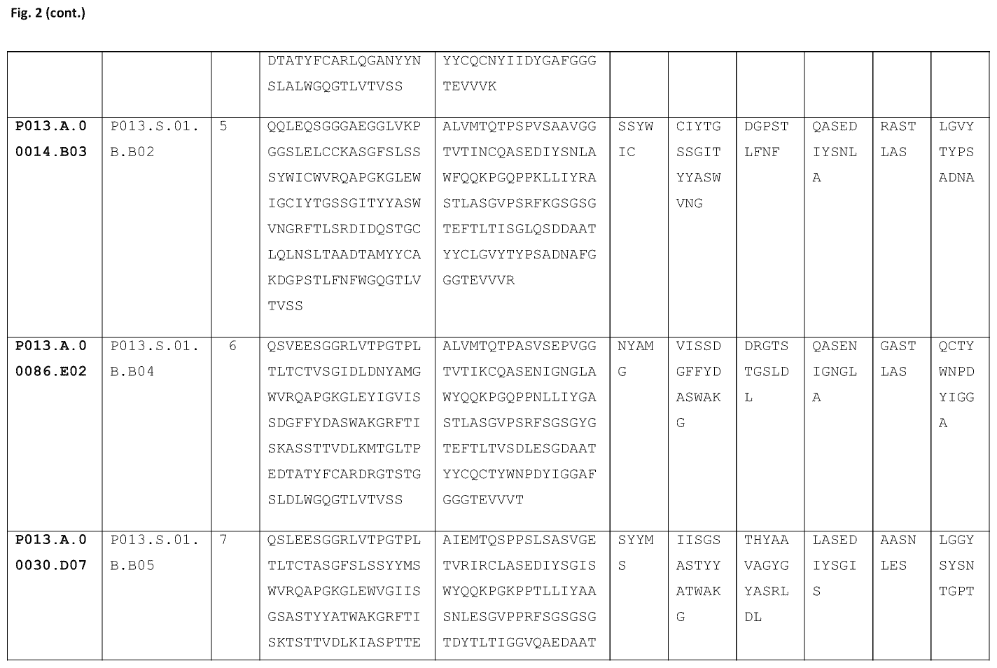

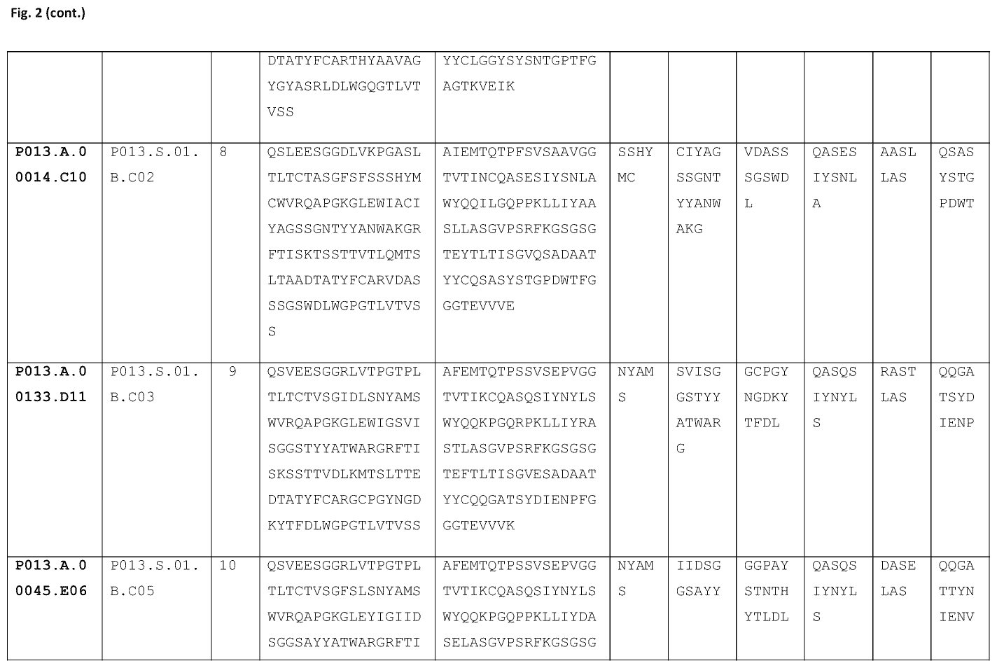

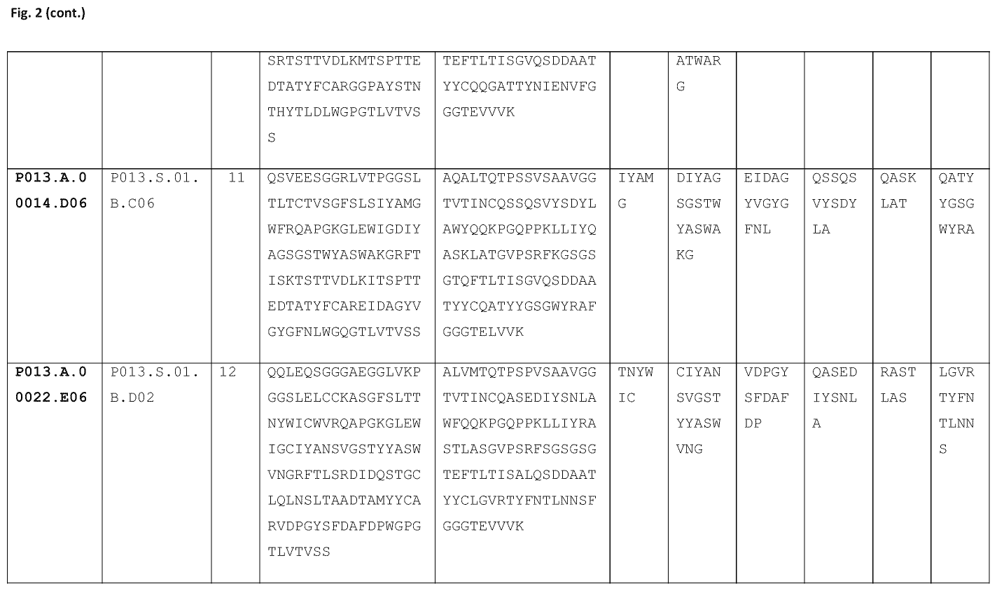

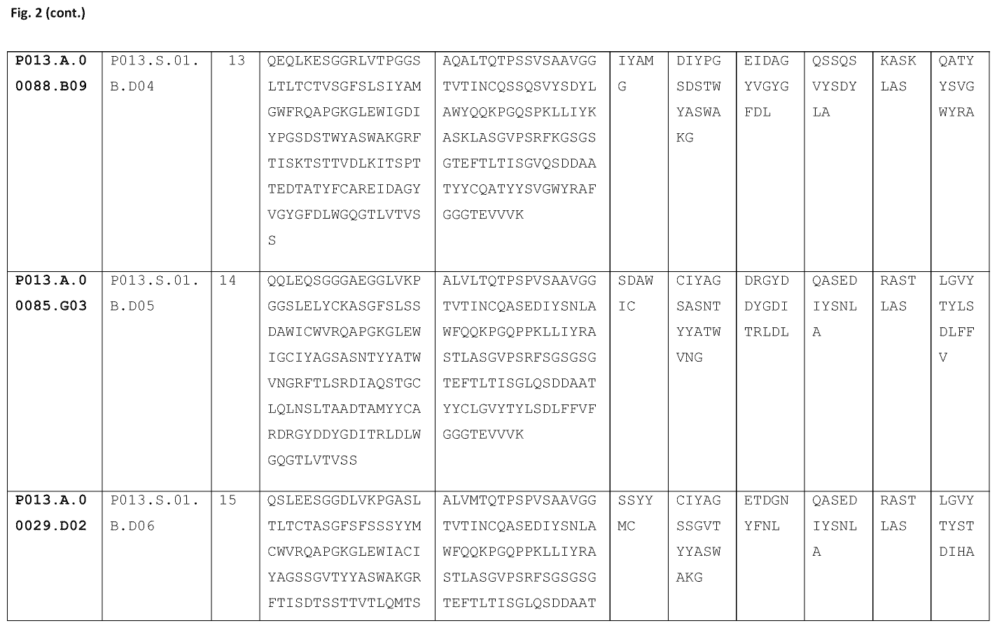

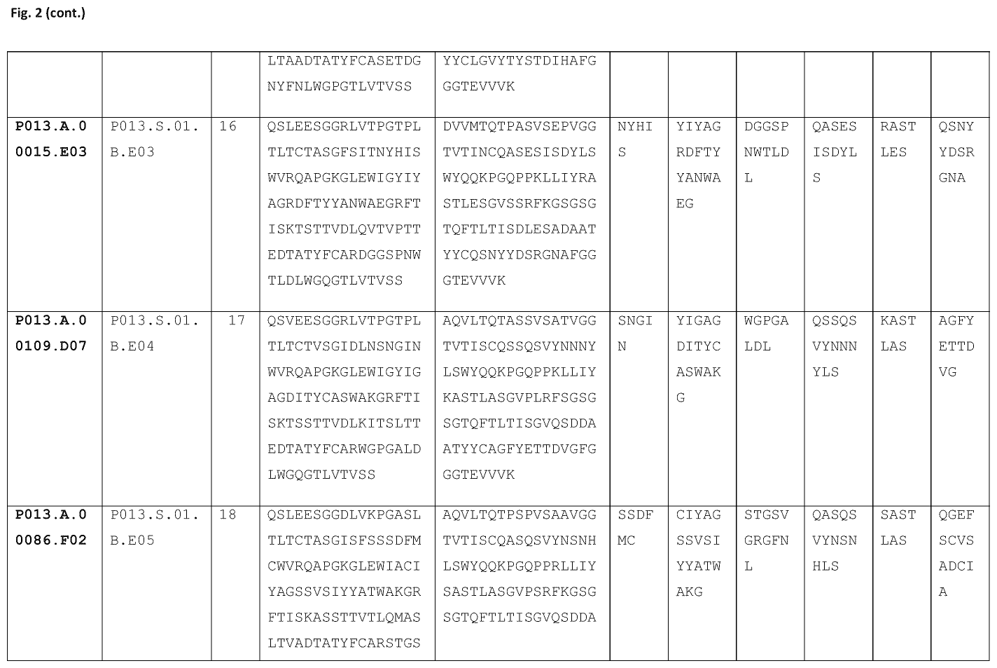

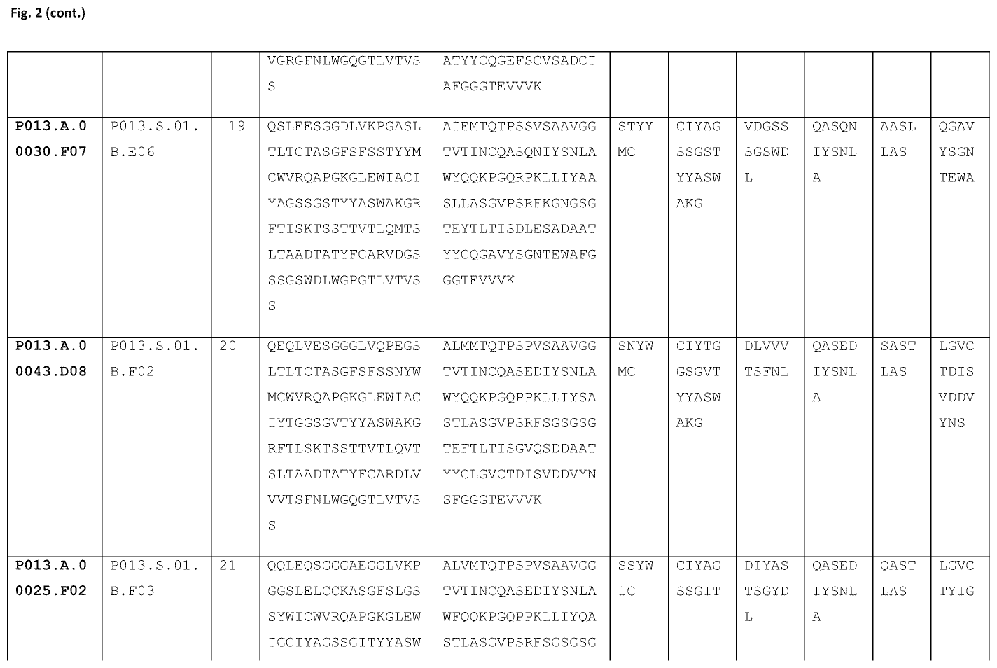

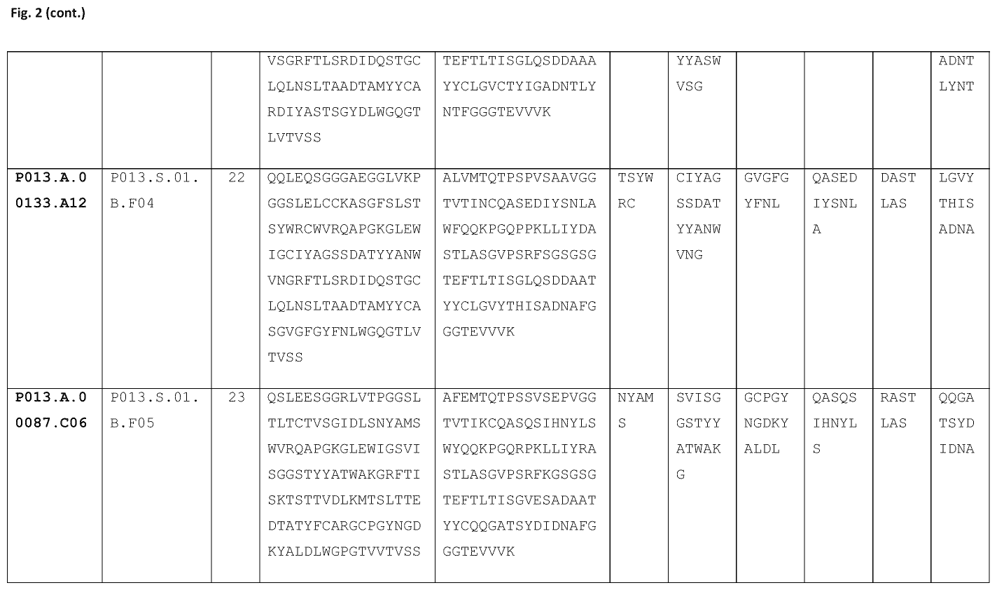

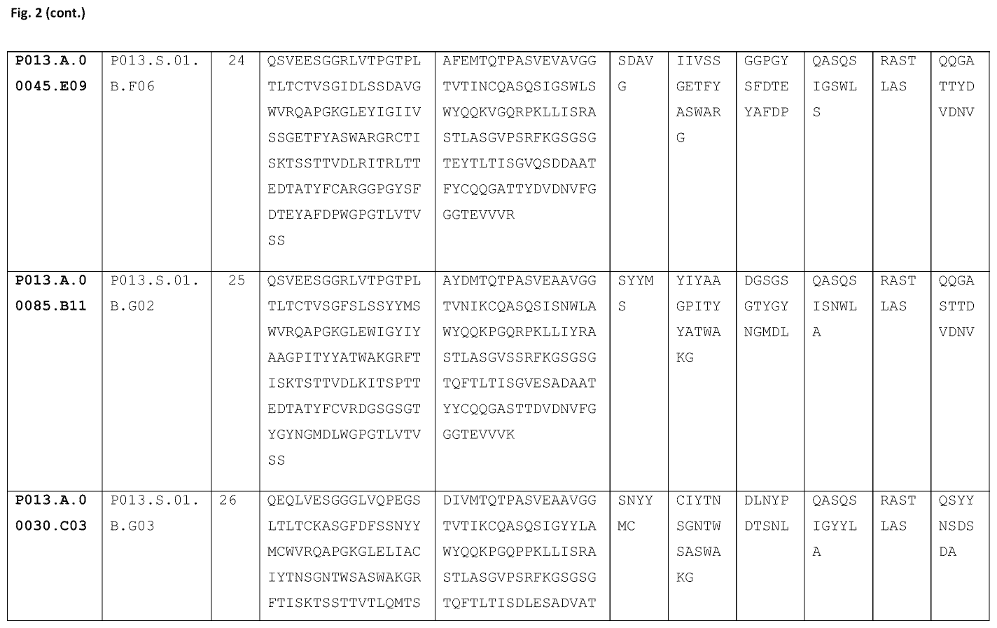

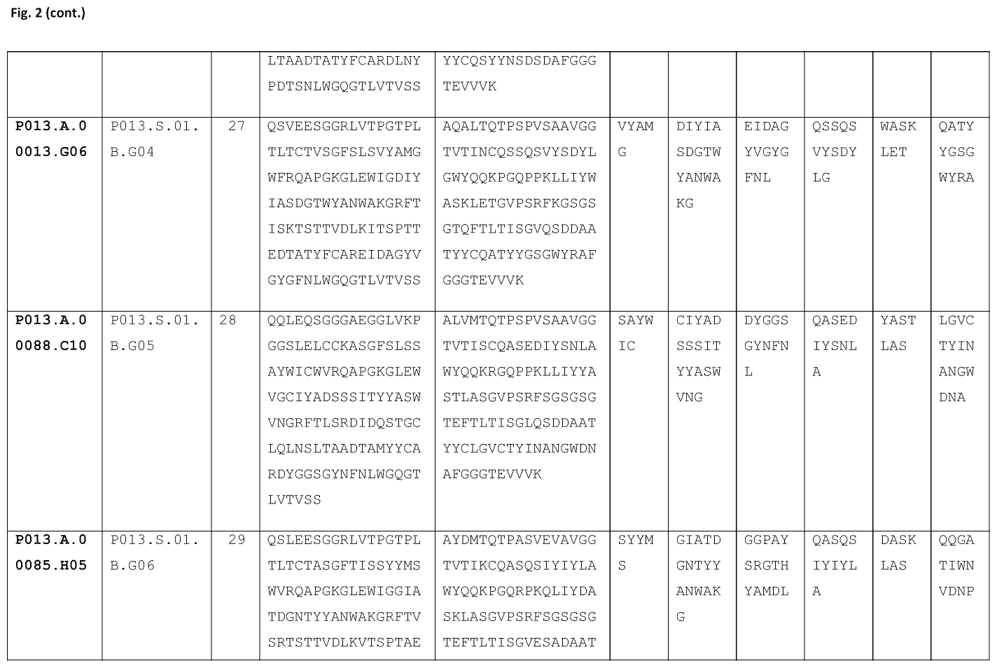

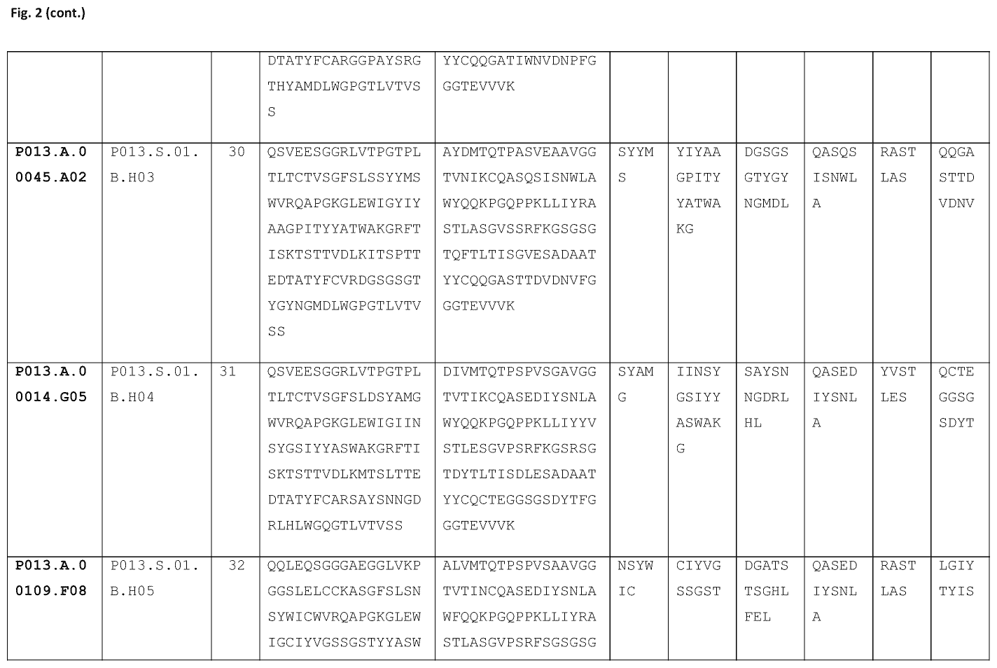

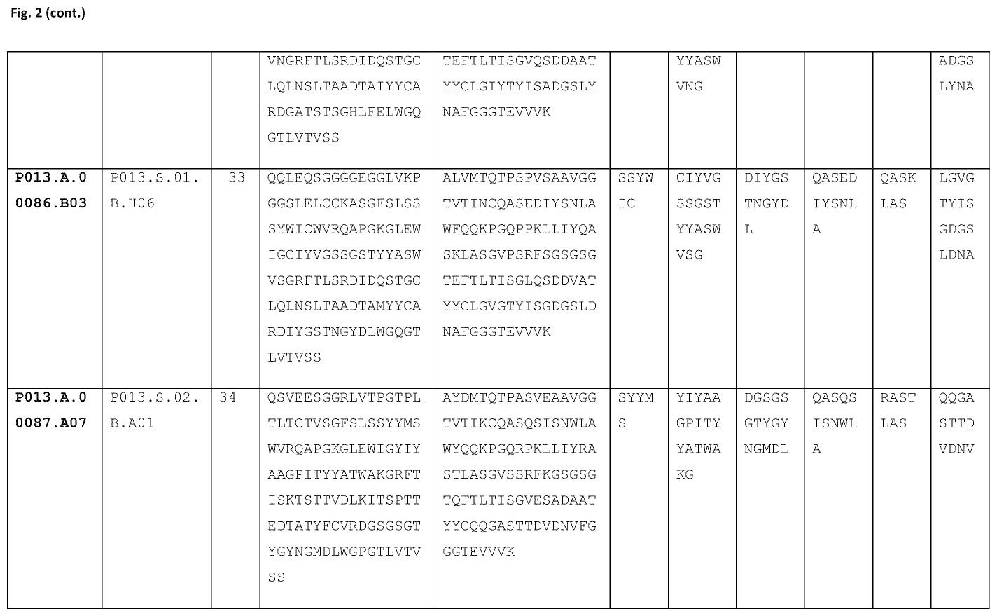

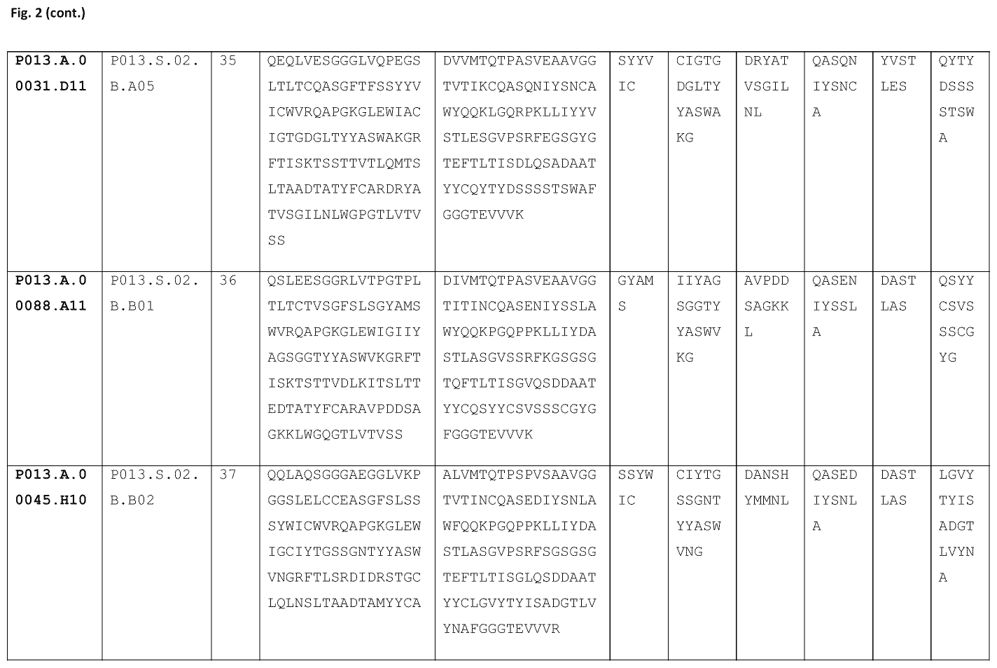

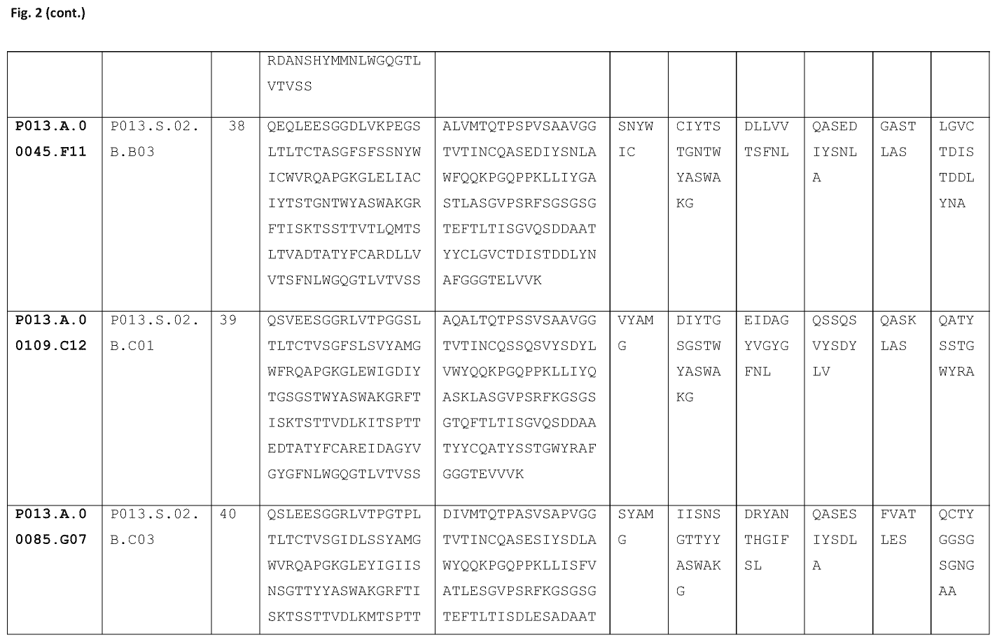

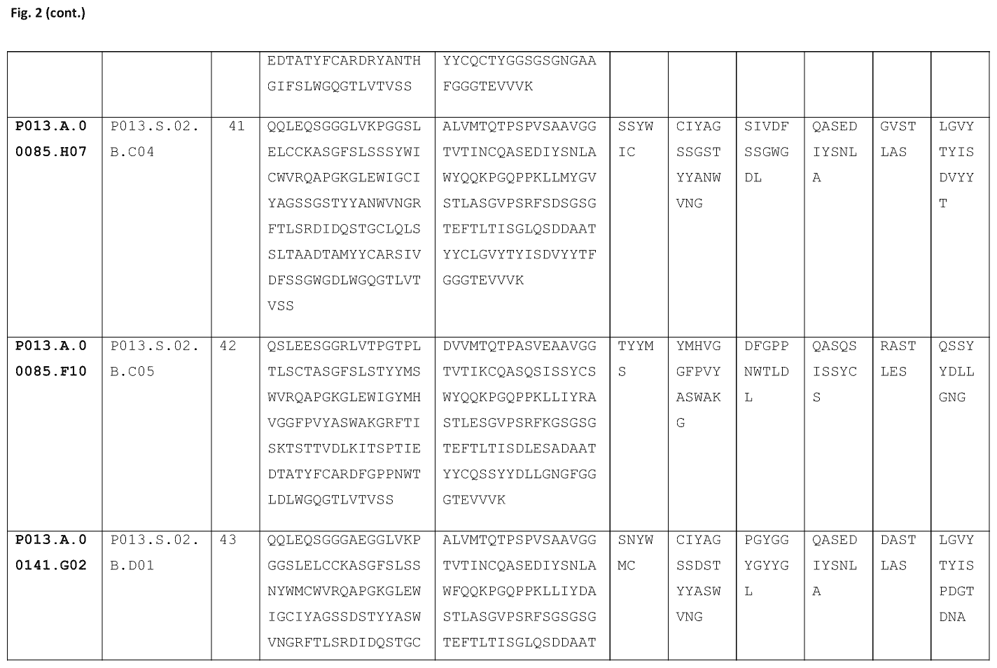

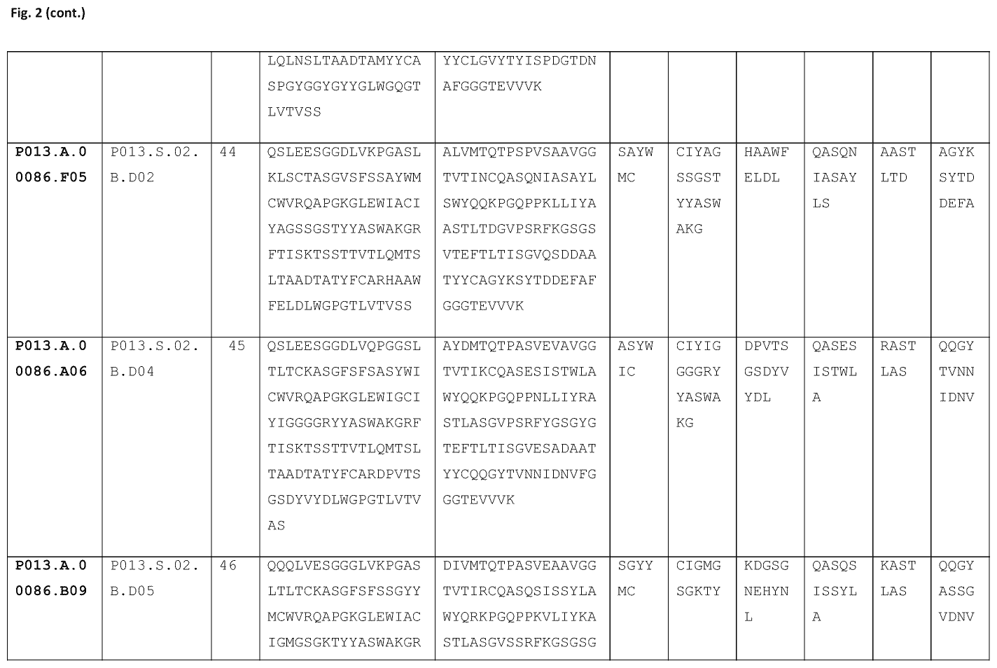

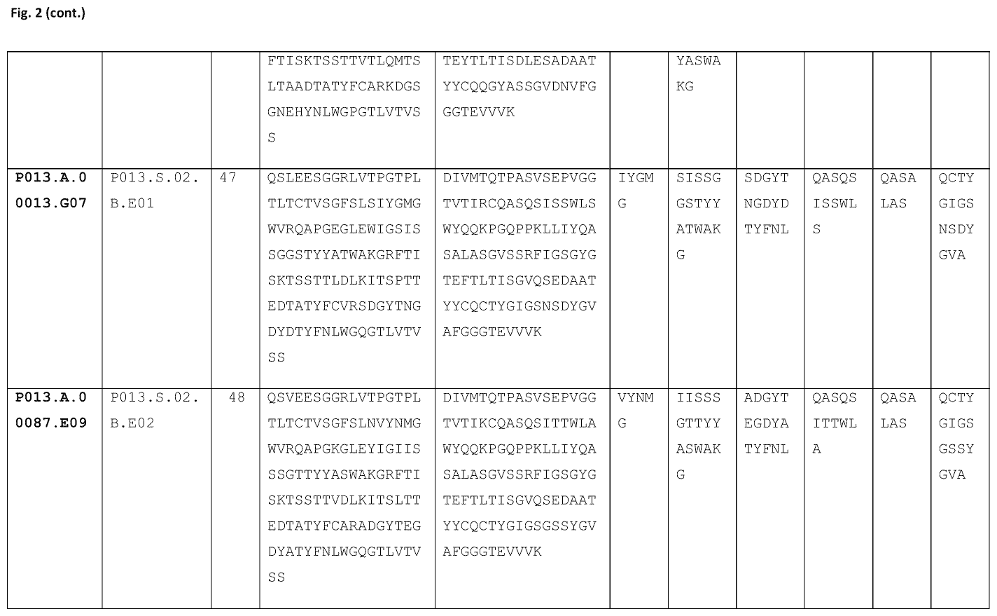

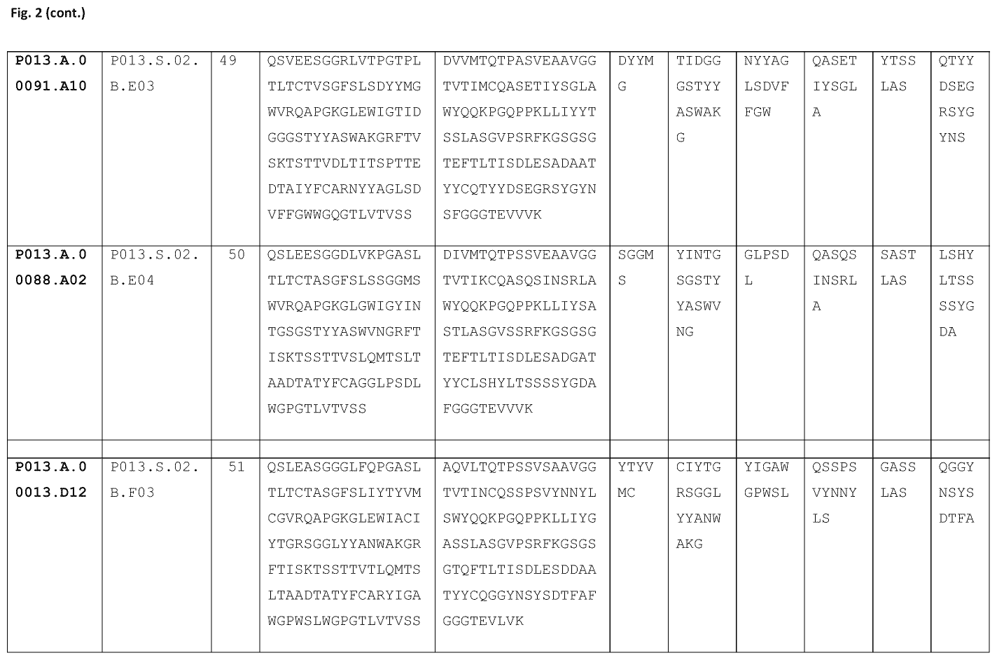

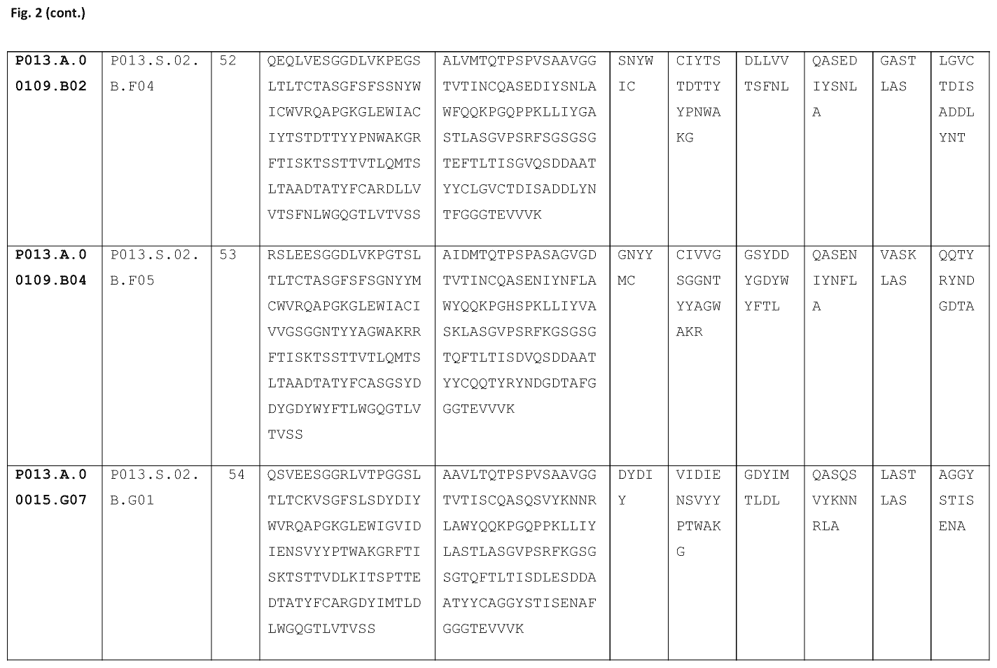

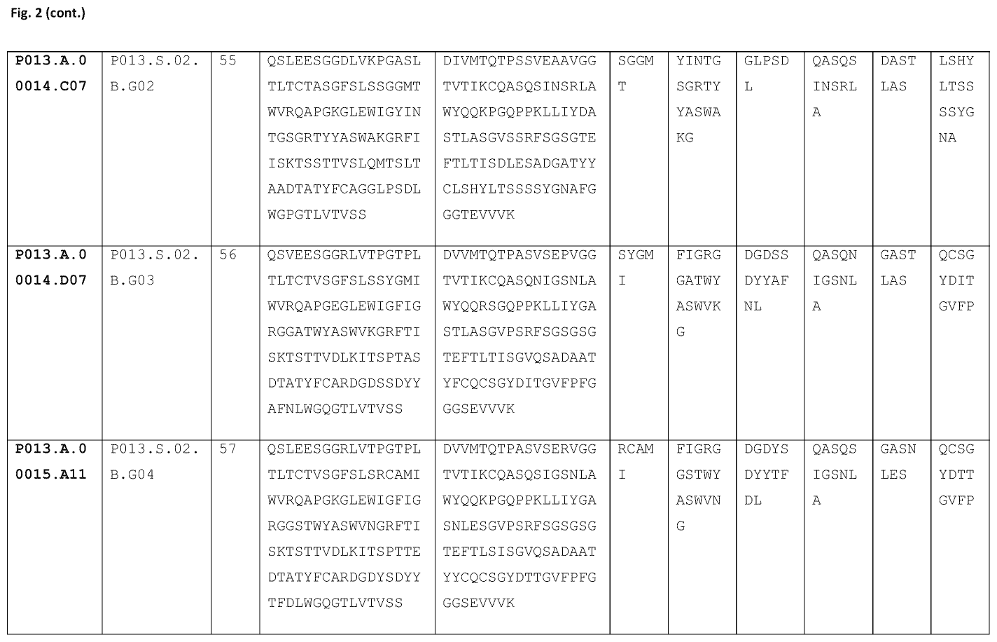

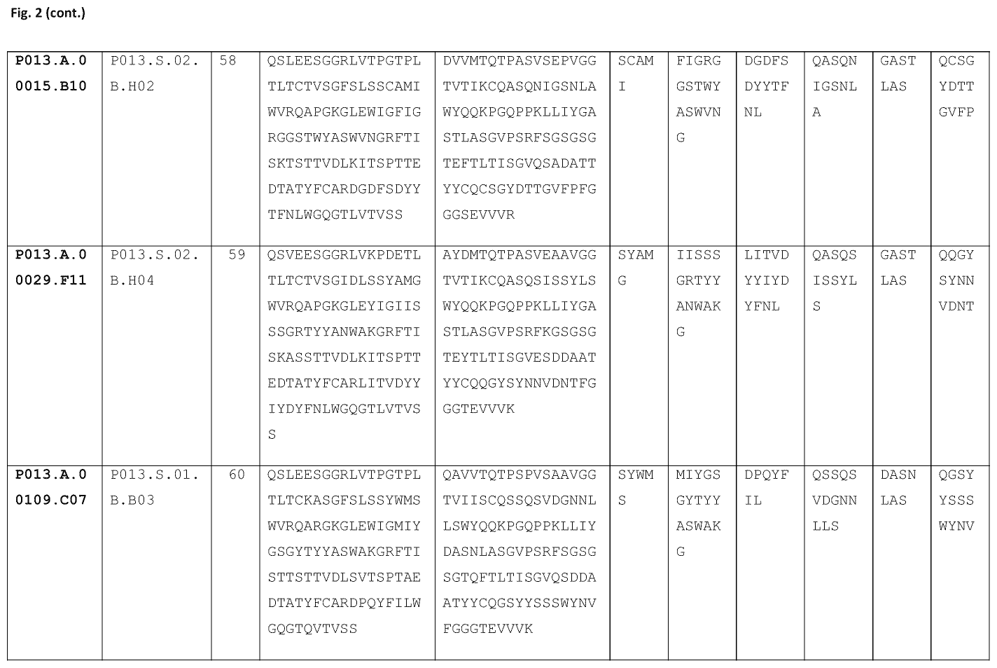

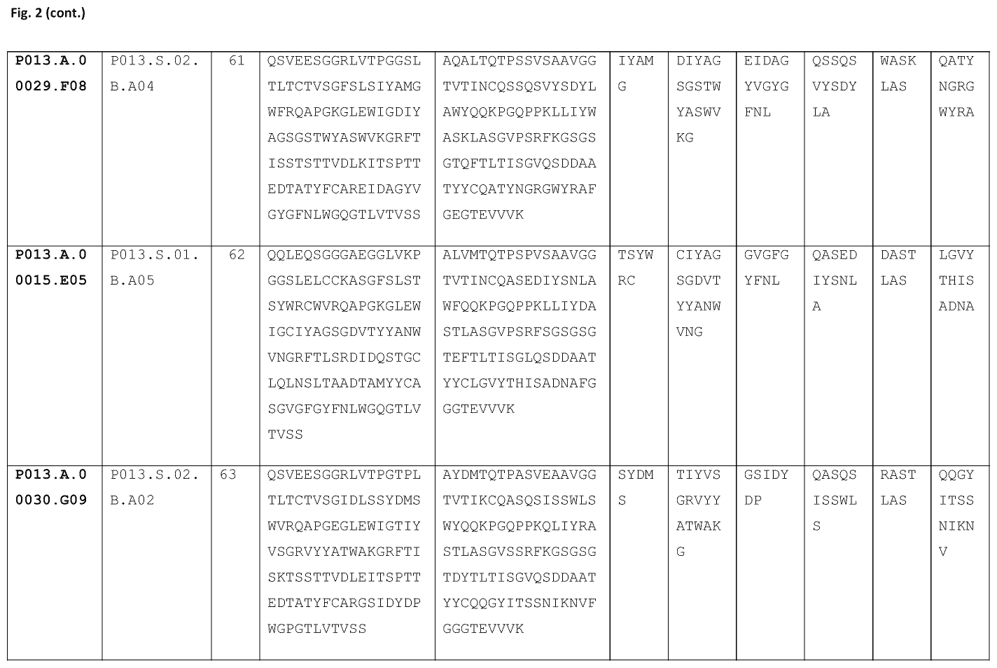

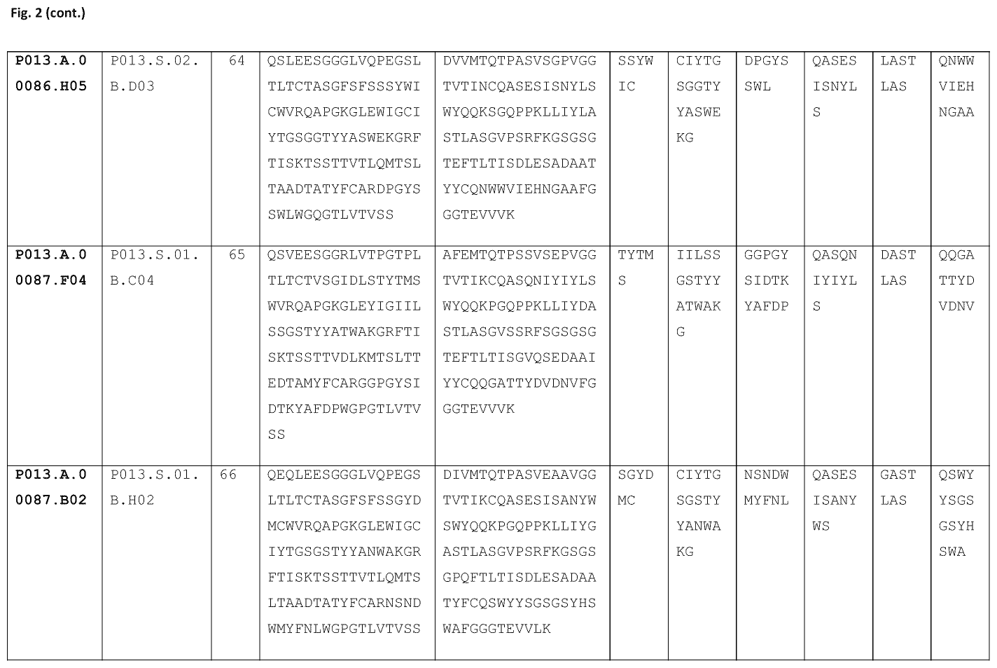

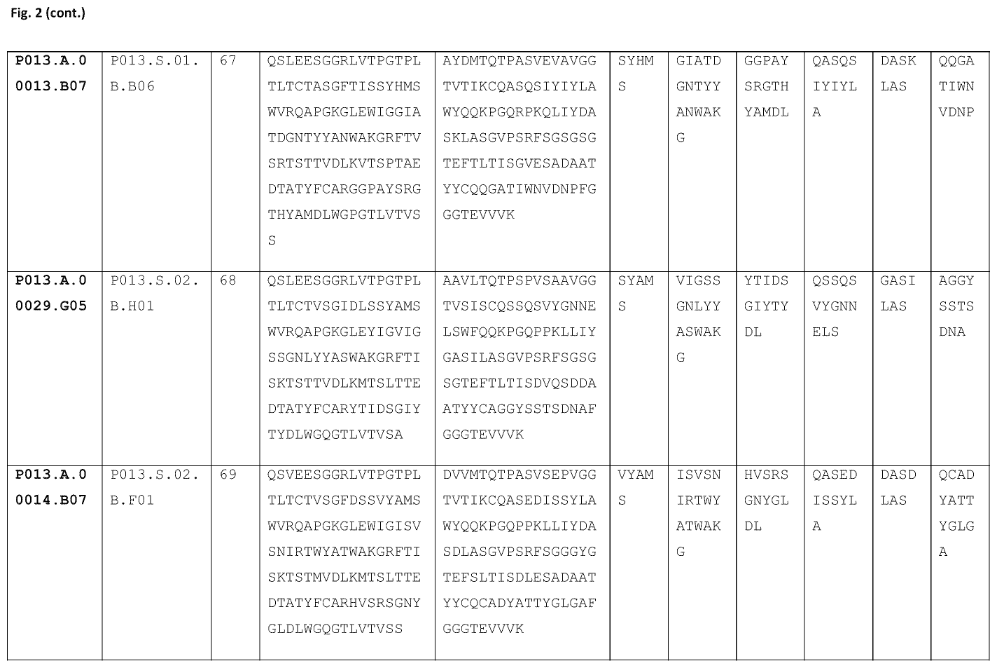

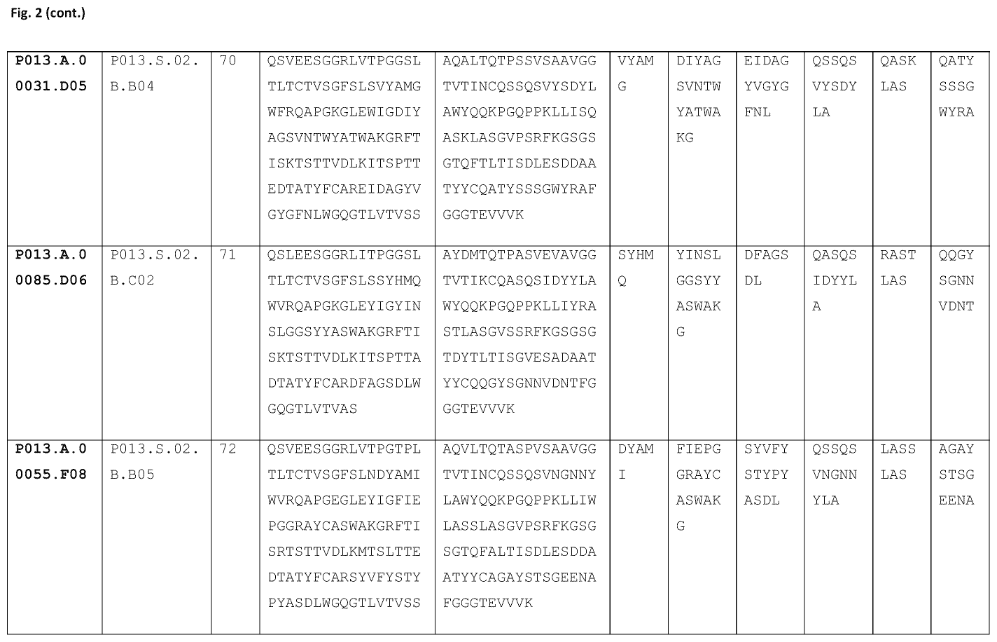

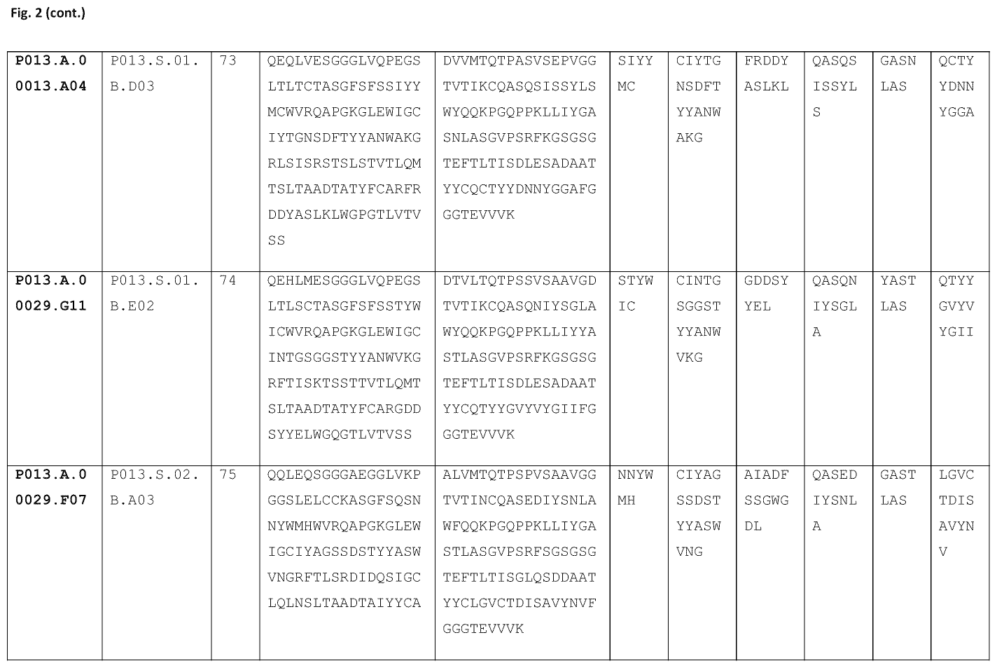

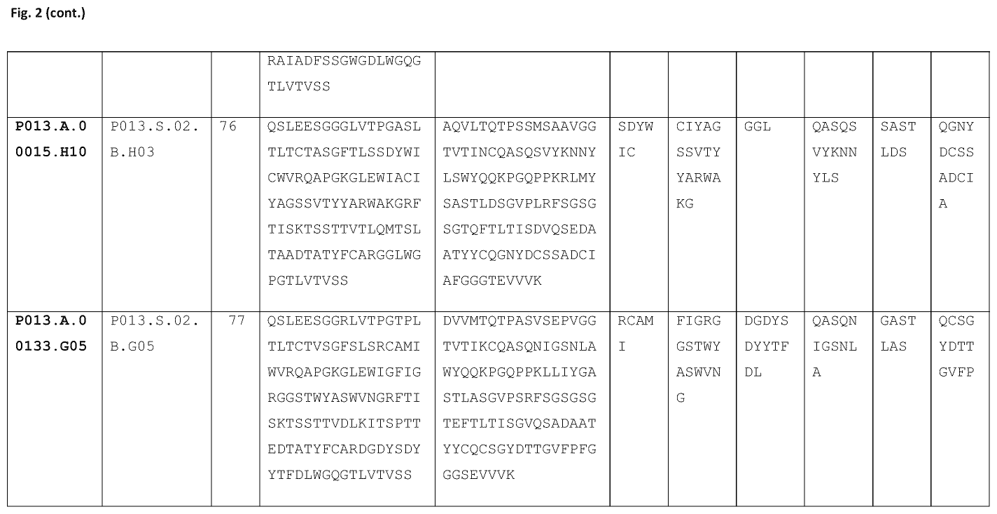

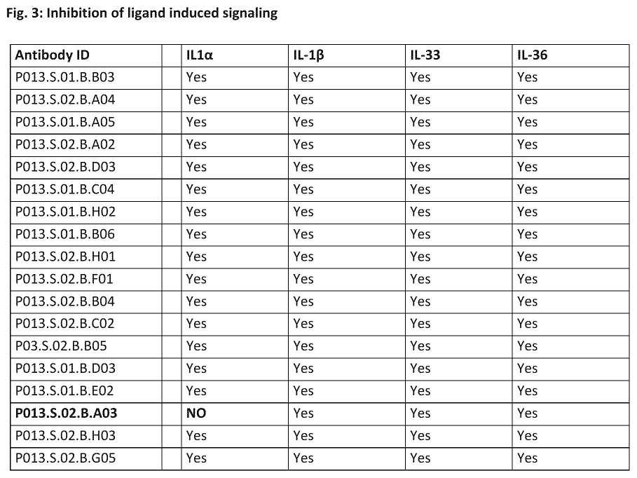

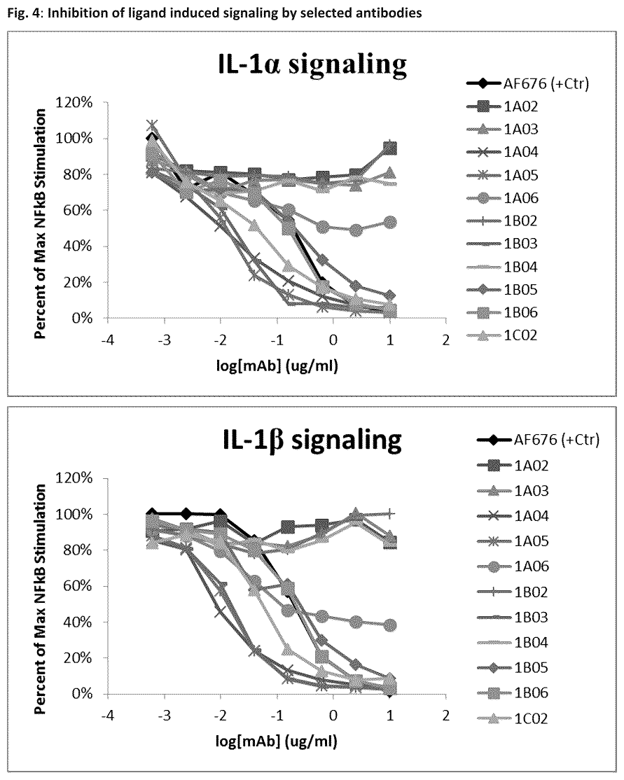

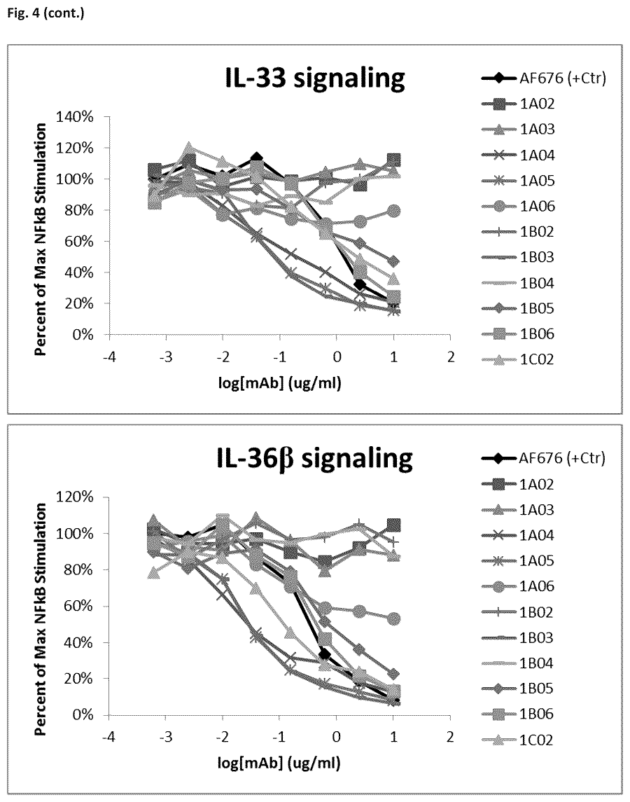

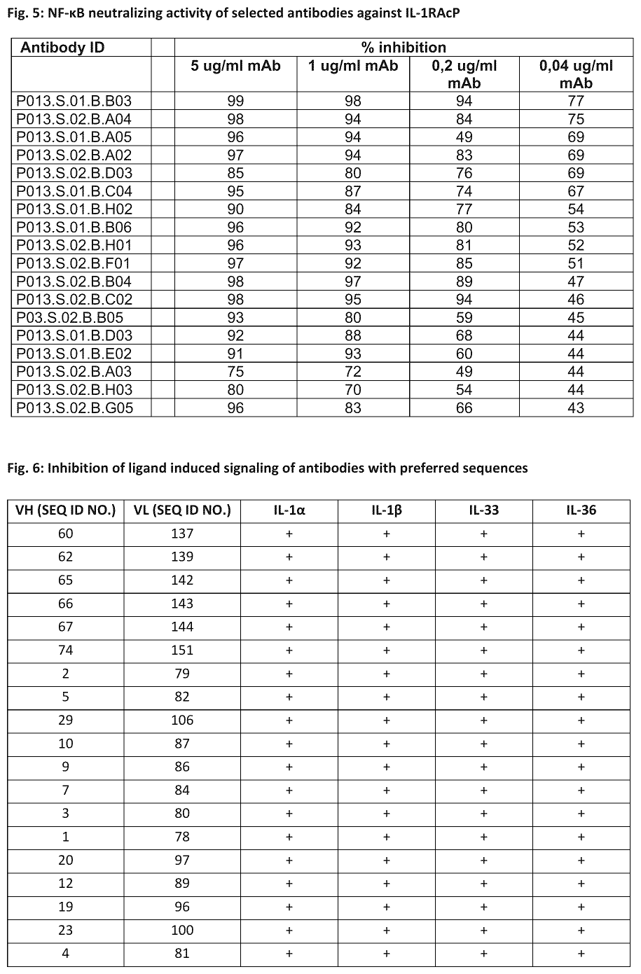

Shown are the results of signaling inhibition experiments for 19 preferred antibodies. Experimental procedures are detailed in examples 8-11.

1-36. (canceled)

37. A method of treating an IL-1 mediated disease in a patient, comprising administering to a patient a pharmaceutically effective amount of the monoclonal antibody, or antigen binding fragment thereof, that specifically binds to human IL-1RAcP.

38. The method of claim 37, wherein the monoclonal antibody is capable of inhibiting IL-1RAcP induced NFkB activity.

39. The method of claim 37, wherein the monoclonal antibody is capable of inhibiting IL-1 alpha, IL-1 beta, IL-33, and/or IL-36 stimulated NFκB activity.

40. The method of claim 37, wherein the monoclonal antibody is capable of binding to murine IL-1RAcP.

41. The method of claim 37, wherein the monoclonal antibody is capable of inhibiting murine IL-1RAcP induced murine NFκB activity.

42. The method of claim 37, wherein the monoclonal antibody is capable of inhibiting NFκB activity stimulated by a complex selected from the group consisting of IL-1β/IL-1R1/IL-1RAcP, IL-1α/IL-1R1/IL-1RAcP, IL-33/ST2/IL-1RAcP, and/or IL-36/IL-36R/IL-1RAcP.

43. The method of claim 37, wherein ADCC activity of the monoclonal antibody is reduced by at least 20% relative to an antibody comprising a wild-type human IgG1 Fc region.

44. The method of claim 37, wherein the monoclonal antibody comprises reduced affinity to the human FcγRIIIA, FcγRIIA, and/or FcγRI relative to an antibody comprising a wild-type human IgG1 Fc region.

45. The method of claim 37, wherein the monoclonal antibody comprises amino acid substitutions at L234A and L235A of the human IgG1 Fc region, or S228P and L235E of the human IgG4 Fc region.

46. The method of claim 37, wherein the monoclonal antibody comprises a heavy chain variable (VH) region that is at least 90% identical to a VH region selected from the group consisting of VH regions of SEQ ID NOs: 1 to 77.

47. The method of claim 37, wherein the monoclonal antibody comprises a light chain variable (VL) region that is at least 90% identical to a VL region selected from the group consisting of VL regions of SEQ ID NOs: 78 to 154.

48. The method of claim 37, wherein the monoclonal antibody comprises a VH region that is at least 90% identical to a VH region of SEQ ID NO: 1+n, and a VL region that is at least 90% identical to a VL region of SEQ ID NO: 78+n, wherein n is a number selected from the group consisting of 0 to 76.

49. The method of claim 37, wherein the monoclonal antibody comprises a VH region amino acid sequence selected from the group consisting of SEQ ID NOs: 1 to 77.

50. The method of claim 37, wherein the monoclonal antibody comprises a VL region amino acid sequence selected from the group consisting of SEQ ID NOs: 78 to 154.

51. The method of claim 37, wherein the monoclonal antibody comprises, a VH region of SEQ ID NO: 1+n, and a VL region of SEQ ID NO: 78+n, wherein n is a number selected from the group consisting of 0 to 76.

52. The method of claim 37, wherein the monoclonal antibody comprises a CDR1H region of SEQ ID NO: 155+n, a CDR2H region of SEQ ID NO: 232+n, and a CDR3H region of SEQ ID NO: 309+n, wherein n is a number selected from the group consisting of 0 to 76.

53. The method of claim 37, wherein the monoclonal antibody comprises a CDR1L region of SEQ ID NO: 386+n, a CDR2L region of SEQ ID NO: 463+n, and a CDR3L region of SEQ ID NO: 540+n, wherein n is a number selected from the group consisting of 0 to 76.

54. The method of claim 37, wherein the monoclonal antibody comprises:
a) a heavy chain variable region (VH) comprising CDR1H, CDR2H and CDR3H, wherein
the CDR1H region comprises an amino acid sequence selected from the group of SEQ ID NO: 155-231;
the CDR2H region comprises an amino acid sequence selected from the group of SEQ ID NO: 232-308; and
the CDR3H region comprises an amino acid sequence selected from the group of SEQ ID NO: 309-385; and
b) a light chain variable region (VL) comprising CDR1L, CDR2L and CDR3L, wherein
the CDR1L region comprises an amino acid sequence selected from the group of SEQ ID NO: 386-462;
the CDRL2 region comprises an amino acid sequence selected from the group of SEQ ID NO: 463-539; and
the CDR3L region comprises an amino acid sequence selected from the group of SEQ ID NO: 540-616.

55. The method of claim 37, wherein the monoclonal antibody is capable of binding to the same epitope as an antibody selected from the group of antibodies consisting of P013.S.01.B.B03, P013.S.01.B.A05, P013.S.01.B.C04, P013.S.01.B.H01, P013.S.01.B.D03, P013.S.01.B.E02, P013.S.02.B.A04, P013.S.02.B.A05, P013.S.02.B.A02, P013.S.02.B.D03, P013.S.02.B.H01, P013.S.02.B.F01, P013.S.02.B.B04, P013.S.02.B.C02, P013.S.02.B.B05, P013.S.02.B.A03, P013.S.02.B.H03, and P013.S.02.B.G05.

56. The method of claim 37, wherein the monoclonal antibody comprises a VH region and a VL region comprising the respective CDR1, CDR2 and CDR3 regions of an antibody selected from the group consisting of antibodies P013.S.01.B.B03, P013.S.01.B.A05, P013.S.01.B.C04, P013.S.01.B.H01, P013.S.01.B.D03, P013.S.01.B.E02, P013.S.02.B.A04, P013.S.02.B.A05, P013.S.02.B.A02, P013.S.02.B.D03, P013.S.02.B.H01, P013.S.02.B.F01, P013.S.02.B.B04, P013.S.02.B.C02, P013.S.02.B.B05, P013.S.02.B.A03, P013.S.02.B.H03, and P013.S.02.B.G05.

57. The method of claim 37, wherein the monoclonal antibody is a rabbit/human chimeric antibody or a humanized antibody.

58. The method of claim 37, wherein the monoclonal antibody comprises a VH region and a VL region comprising the respective CDR1, CDR2 and CDR3 regions of an antibody selected from the group consisting of antibodies listed in table 3.


SEQUENCE LISTING (616 sequences)

SEQ ID NO: 1 - protein, 121 aa, Oryctolagus cuniculus
QSVEESGGRLVTPGTPLTLTCTVSGIDLSSYAMGWVRQAPGKGLEYIGVITSSATTYYASWAKGRFTISKTSSTTVDLRVTSLTTEDTATYFCARGGPGYSTNTHYAFDPWGPGTLVTVSS

SEQ ID NO: 2 - protein, 118 aa, Oryctolagus cuniculus
QEQLEESGGDLVQPEGSLTVTCTASGFSFSFGYYMCWVRQAPGKGLEWIACIYGDSSDTLYANWAKGRFTVSKTSSTTVTLQMTSLTAADTATYFCARYPGGSYYNLWGPGTRVTVSS

SEQ ID NO: 3 - protein, 121 aa, Oryctolagus cuniculus
QEQLEESGGGLVKPGGTLTLTCKASGIDFSSYYYMCWVRQAPGKGLEWIACIFIGYGDVTWYASWAKGRFTISKASSTTVTLQMTSLTAADTATYFCARALGSSGYRVNLWGPGTLVTVSS

SEQ ID NO: 4 - protein, 117 aa, Oryctolagus cuniculus
QSLEESGGRLVTPGTPLTLSCKVSGFSLSSYDMSWVRQTPGKGLEWIGTIYIGGTTAYASWPKGRFTISKTSTTVDLKITSPTKEDTATYFCARLQGANYYNSLALWGQGTLVTVSS

SEQ ID NO: 5 - protein, 123 aa, Oryctolagus cuniculus
QQLEQSGGGAEGGLVKPGGSLELCCKASGFSLSSSYWICWVRQAPGKGLEWIGCIYTGSSGITYYASWVNGRFTLSRDIDQSTGCLQLNSLTAADTAMYYCAKDGPSTLFNFWGQGTLVTVSS

SEQ ID NO: 6 - protein, 117 aa, Oryctolagus cuniculus
QSVEESGGRLVTPGTPLTLTCTVSGIDLDNYAMGWVRQAPGKGLEYIGVISSDGFFYDASWAKGRFTISKASSTTVDLKMTGLTPEDTATYFCARDRGTSTGSLDLWGQGTLVTVSS

SEQ ID NO: 7 - protein, 122 aa, Oryctolagus cuniculus
QSLEESGGRLVTPGTPLTLTCTASGFSLSSYYMSWVRQAPGKGLEWVGIISGSASTYYATWAKGRFTISKTSTTVDLKIASPTTEDTATYFCARTHYAAVAGYGYASRLDLWGQGTLVTVSS

SEQ ID NO: 8 - protein, 120 aa, Oryctolagus cuniculus
QSLEESGGDLVKPGASLTLTCTASGFSFSSSHYMCWVRQAPGKGLEWIACIYAGSSGNTYYANWAKGRFTISKTSSTTVTLQMTSLTAADTATYFCARVDASSSGSWDLWGPGTLVTVSS

SEQ ID NO: 9 - protein, 119 aa, Oryctolagus cuniculus
QSVEESGGRLVTPGTPLTLTCTVSGIDLSNYAMSWVRQAPGKGLEWIGSVISGGSTYYATWARGRFTISKSSTTVDLKMTSLTTEDTATYFCARGCPGYNGDKYTFDLWGPGTLVTVSS

SEQ ID NO: 10 - protein, 120 aa, Oryctolagus cuniculus
QSVEESGGRLVTPGTPLTLTCTVSGFSLSNYAMSWVRQAPGKGLEYIGIIDSGGSAYYATWARGRFTISRTSTTVDLKMTSPTTEDTATYFCARGGPAYSTNTHYTLDLWGPGTLVTVSS

SEQ ID NO: 11 - protein, 119 aa, Oryctolagus cuniculus
QSVEESGGRLVTPGGSLTLTCTVSGFSLSIYAMGWFRQAPGKGLEWIGDIYAGSGSTWYASWAKGRFTISKTSTTVDLKITSPTTEDTATYFCAREIDAGYVGYGFNLWGQGTLVTVSS

SEQ ID NO: 12 - protein, 126 aa, Oryctolagus cuniculus
QQLEQSGGGAEGGLVKPGGSLELCCKASGFSLTTNYWICWVRQAPGKGLEWIGCIYANSVGSTYYASWVNGRFTLSRDIDQSTGCLQLNSLTAADTAMYYCARVDPGYSFDAFDPWGPGTLVTVSS

SEQ ID NO: 13 - protein, 120 aa, Oryctolagus cuniculus
QEQLKESGGRLVTPGGSLTLTCTVSGFSLSIYAMGWFRQAPGKGLEWIGDIYPGSDSTWYASWAKGRFTISKTSTTVDLKITSPTTEDTATYFCAREIDAGYVGYGFDLWGQGTLVTVSS

SEQ ID NO: 14 - protein, 129 aa, Oryctolagus cuniculus
QQLEQSGGGAEGGLVKPGGSLELYCKASGFSLSSDAWICWVRQAPGKGLEWIGCIYAGSASNTYYATWVNGRFTLSRDIAQSTGCLQLNSLTAADTAMYYCARDRGYDDYGDITRLDLWGQGTLVTVSS

SEQ ID NO: 15 - protein, 118 aa, Oryctolagus cuniculus
QSLEESGGDLVKPGASLTLTCTASGFSFSSSYYMCWVRQAPGKGLEWIACIYAGSSGVTYYASWAKGRFTISDTSSTTVTLQMTSLTAADTATYFCASETDGNYFNLWGPGTLVTVSS

SEQ ID NO: 16 - protein, 117 aa, Oryctolagus cuniculus
QSLEESGGRLVTPGTPLTLTCTASGFSITNYHISWVRQAPGKGLEWIGYIYAGRDFTYYANWAEGRFTISKTSTTVDLQVTVPTTEDTATYFCARDGGSPNWTLDLWGQGTLVTVSS

SEQ ID NO: 17 - protein, 114 aa, Oryctolagus cuniculus
QSVEESGGRLVTPGTPLTLTCTVSGIDLNSNGINWVRQAPGKGLEWIGYIGAGDITYCASWAKGRFTISKTSSTTVDLKITSLTTEDTATYFCARWGPGALDLWGQGTLVTVSS

SEQ ID NO: 18 - protein, 120 aa, Oryctolagus cuniculus
QSLEESGGDLVKPGASLTLTCTASGISFSSSDFMCWVRQAPGKGLEWIACIYAGSSVSIYYATWAKGRFTISKASSTTVTLQMASLTVADTATYFCARSTGSVGRGFNLWGQGTLVTVSS

SEQ ID NO: 19 - protein, 120 aa, Oryctolagus cuniculus
QSLEESGGDLVKPGASLTLTCTASGFSFSSTYYMCWVRQAPGKGLEWIACIYAGSSGSTYYASWAKGRFTISKTSSTTVTLQMTSLTAADTATYFCARVDGSSSGSWDLWGPGTLVTVSS

SEQ ID NO: 20 - protein, 120 aa, Oryctolagus cuniculus
QEQLVESGGGLVQPEGSLTLTCTASGFSFSSNYWMCWVRQAPGKGLEWIACIYTGGSGVTYYASWAKGRFTLSKTSSTTVTLQVTSLTAADTATYFCARDLVVVTSFNLWGQGTLVTVSS

SEQ ID NO: 21 - protein, 125 aa, Oryctolagus cuniculus
QQLEQSGGGAEGGLVKPGGSLELCCKASGFSLGSSYWICWVRQAPGKGLEWIGCIYAGSSGITYYASWVSGRFTLSRDIDQSTGCLQLNSLTAADTAMYYCARDIYASTSGYDLWGQGTLVTVSS

SEQ ID NO: 22 - protein, 123 aa, Oryctolagus cuniculus
QQLEQSGGGAEGGLVKPGGSLELCCKASGFSLSTSYWRCWVRQAPGKGLEWIGCIYAGSSDATYYANWVNGRFTLSRDIDQSTGCLQLNSLTAADTAMYYCASGVGFGYFNLWGQGTLVTVSS

SEQ ID NO: 23 - protein, 119 aa, Oryctolagus cuniculus
QSLEESGGRLVTPGGSLTLTCTVSGIDLSNYAMSWVRQAPGKGLEWIGSVISGGSTYYATWAKGRFTISKTSTTVDLKMTSLTTEDTATYFCARGCPGYNGDKYALDLWGPGTVVTVSS

SEQ ID NO: 24 - protein, 121 aa, Oryctolagus cuniculus
QSVEESGGRLVTPGTPLTLTCTVSGIDLSSDAVGWVRQAPGKGLEYIGIIVSSGETFYASWARGRCTISKTSSTTVDLRITRLTTEDTATYFCARGGPGYSFDTEYAFDPWGPGTLVTVSS

SEQ ID NO: 25 - protein, 121 aa, Oryctolagus cuniculus
QSVEESGGRLVTPGTPLTLTCTVSGFSLSSYYMSWVRQAPGKGLEWIGYIYAAGPITYYATWAKGRFTISKTSTTVDLKITSPTTEDTATYFCVRDGSGSGTYGYNGMDLWGPGTLVTVSS

SEQ ID NO: 26 - protein, 119 aa, Oryctolagus cuniculus
QEQLVESGGGLVQPEGSLTLTCKASGFDFSSNYYMCWVRQAPGKGLELIACIYTNSGNTWSASWAKGRFTISKTSSTTVTLQMTSLTAADTATYFCARDLNYPDTSNLWGQGTLVTVSS

SEQ ID NO: 27 - protein, 119 aa, Oryctolagus cuniculus
QSVEESGGRLVTPGTPLTLTCTVSGFSLSVYAMGWFRQAPGKGLEWIGDIYIASDGTWYANWAKGRFTISKTSTTVDLKITSPTTEDTATYFCAREIDAGYVGYGFNLWGQGTLVTVSS

SEQ ID NO: 28 - protein, 125 aa, Oryctolagus cuniculus
QQLEQSGGGAEGGLVKPGGSLELCCKASGFSLSSAYWICWVRQAPGKGLEWVGCIYADSSSITYYASWVNGRFTLSRDIDQSTGCLQLNSLTAADTAMYYCARDYGGSGYNFNLWGQGTLVTVSS

SEQ ID NO: 29 - protein, 120 aa, Oryctolagus cuniculus
QSLEESGGRLVTPGTPLTLTCTASGFTISSYYMSWVRQAPGKGLEWIGGIATDGNTYYANWAKGRFTVSRTSTTVDLKVTSPTAEDTATYFCARGGPAYSRGTHYAMDLWGPGTLVTVSS

SEQ ID NO: 30 - protein, 121 aa, Oryctolagus cuniculus; identical to SEQ ID NO: 25

SEQ ID NO: 31 - protein, 117 aa, Oryctolagus cuniculus
QSVEESGGRLVTPGTPLTLTCTVSGFSLDSYAMGWVRQAPGKGLEWIGIINSYGSIYYASWAKGRFTISKTSTTVDLKMTSLTTEDTATYFCARSAYSNNGDRLHLWGQGTLVTVSS

SEQ ID NO: 32 - protein, 127 aa, Oryctolagus cuniculus
QQLEQSGGGAEGGLVKPGGSLELCCKASGFSLSNSYWICWVRQAPGKGLEWIGCIYVGSSGSTYYASWVNGRFTLSRDIDQSTGCLQLNSLTAADTAIYYCARDGATSTSGHLFELWGQGTLVTVSS

SEQ ID NO: 33 - protein, 125 aa, Oryctolagus cuniculus
QQLEQSGGGGEGGLVKPGGSLELCCKASGFSLSSSYWICWVRQAPGKGLEWIGCIYVGSSGSTYYASWVSGRFTLSRDIDQSTGCLQLNSLTAADTAMYYCARDIYGSTNGYDLWGQGTLVTVSS

SEQ ID NO: 34 - protein, 121 aa, Oryctolagus cuniculus; identical to SEQ ID NO: 25

SEQ ID NO: 35 - protein, 121 aa, Oryctolagus cuniculus
QEQLVESGGGLVQPEGSLTLTCQASGFTFSSYYVICWVRQAPGKGLEWIACIGTGDGLTYYASWAKGRFTISKTSSTTVTLQMTSLTAADTATYFCARDRYATVSGILNLWGPGTLVTVSS

SEQ ID NO: 36 - protein, 117 aa, Oryctolagus cuniculus
QSLEESGGRLVTPGTPLTLTCTVSGFSLSGYAMSWVRQAPGKGLEWIGIIYAGSGGTYYASWVKGRFTISKTSTTVDLKITSLTTEDTATYFCARAVPDDSAGKKLWGQGTLVTVSS

SEQ ID NO: 37 - protein, 124 aa, Oryctolagus cuniculus
QQLAQSGGGAEGGLVKPGGSLELCCEASGFSLSSSYWICWVRQAPGKGLEWIGCIYTGSSGNTYYASWVNGRFTLSRDIDRSTGCLQLNSLTAADTAMYYCARDANSHYMMNLWGQGTLVTVSS

SEQ ID NO: 38 - protein, 119 aa, Oryctolagus cuniculus
QEQLEESGGDLVKPEGSLTLTCTASGFSFSSNYWICWVRQAPGKGLELIACIYTSTGNTWYASWAKGRFTISKTSSTTVTLQMTSLTVADTATYFCARDLLVVTSFNLWGQGTLVTVSS

SEQ ID NO: 39 - protein, 119 aa, Oryctolagus cuniculus
QSVEESGGRLVTPGGSLTLTCTVSGFSLSVYAMGWFRQAPGKGLEWIGDIYTGSGSTWYASWAKGRFTISKTSTTVDLKITSPTTEDTATYFCAREIDAGYVGYGFNLWGQGTLVTVSS

SEQ ID NO: 40 - protein, 118 aa, Oryctolagus cuniculus
QSLEESGGRLVTPGTPLTLTCTVSGIDLSSYAMGWVRQAPGKGLEYIGIISNSGTTYYASWAKGRFTISKTSSTTVDLKMTSPTTEDTATYFCARDRYANTHGIFSLWGQGTLVTVSS

SEQ ID NO: 41 - protein, 122 aa, Oryctolagus cuniculus
QQLEQSGGGLVKPGGSLELCCKASGFSLSSSYWICWVRQAPGKGLEWIGCIYAGSSGSTYYANWVNGRFTLSRDIDQSTGCLQLSSLTAADTAMYYCARSIVDFSSGWGDLWGQGTLVTVSS

SEQ ID NO: 42 - protein, 116 aa, Oryctolagus cuniculus
QSLEESGGRLVTPGTPLTLSCTASGFSLSTYYMSWVRQAPGKGLEWIGYMHVGGFPVYASWAKGRFTISKTSTTVDLKITSPTIEDTATYFCARDFGPPNWTLDLWGQGTLVTVSS

SEQ ID NO: 43 - protein, 125 aa, Oryctolagus cuniculus
QQLEQSGGGAEGGLVKPGGSLELCCKASGFSLSSNYWMCWVRQAPGKGLEWIGCIYAGSSDSTYYASWVNGRFTLSRDIDQSTGCLQLNSLTAADTAMYYCASPGYGGYGYYGLWGQGTLVTVSS

SEQ ID NO: 44 - protein, 118 aa, Oryctolagus cuniculus
QSLEESGGDLVKPGASLKLSCTASGVSFSSAYWMCWVRQAPGKGLEWIACIYAGSSGSTYYASWAKGRFTISKTSSTTVTLQMTSLTAADTATYFCARHAAWFELDLWGPGTLVTVSS

SEQ ID NO: 45 - protein, 121 aa, Oryctolagus cuniculus
QSLEESGGDLVQPGGSLTLTCKASGFSFSASYWICWVRQAPGKGLEWIGCIYIGGGGRYYASWAKGRFTISKTSSTTVTLQMTSLTAADTATYFCARDPVTSGSDYVYDLWGPGTLVTVAS

SEQ ID NO: 46 - protein, 120 aa, Oryctolagus cuniculus
QQQLVESGGGLVKPGASLTLTCKASGFSFSSGYYMCWVRQAPGKGLEWIACIGMGSGKTYYASWAKGRFTISKTSSTTVTLQMTSLTAADTATYFCARKDGSGNEHYNLWGPGTLVTVSS

SEQ ID NO: 47 - protein, 120 aa, Oryctolagus cuniculus
QSLEESGGRLVTPGTPLTLTCTVSGFSLSIYGMGWVRQAPGEGLEWIGSISSGGSTYYATWAKGRFTISKTSSTTLDLKITSPTTEDTATYFCVRSDGYTNGDYDTYFNLWGQGTLVTVS

SEQ ID NO: 48 - protein, 121 aa, Oryctolagus cuniculus
QSVEESGGRLVTPGTPLTLTCTVSGFSLNVYNMGWVRQAPGKGLEYIGIISSSGTTYYASWAKGRFTISKTSSTTVDLKITSLTTEDTATYFCARADGYTEGDYATYFNLWGQGTLVTVSS

SEQ ID NO: 49 - protein, 118 aa, Oryctolagus cuniculus
QSVEESGGRLVTPGTPLTLTCTVSGFSLSDYYMGWVRQAPGKGLEWIGTIDGGGSTYYASWAKGRFTVSKTSTTVDLTITSPTTEDTAIYFCARNYYAGLSDVFFGWWGQGTLVTVSS

SEQ ID NO: 50 - protein, 113 aa, Oryctolagus cuniculus
QSLEESGGDLVKPGASLTLTCTASGFSLSSGGMSWVRQAPGKGLGWIGYINTGSGSTYYASWVNGRFTISKTSSTTVSLQMTSLTAADTATYFCAGGLPSDLWGPGTLVTVSS

SEQ ID NO: 51 - protein, 119 aa, Oryctolagus cuniculus
QSLEASGGGLFQPGASLTLTCTASGFSLIYTYVMCGVRQAPGKGLEWIACIYTGRSGGLYYANWAKGRFTISKTSSTTVTLQMTSLTAADTATYFCARYIGAWGPWSLWGPGTLVTVSS

SEQ ID NO: 52 - protein, 119 aa, Oryctolagus cuniculus
QEQLVESGGDLVKPEGSLTLTCTASGFSFSSNYWICWVRQAPGKGLEWIACIYTSTDTTYYPNWAKGRFTISKTSSTTVTLQMTSLTAADTATYFCARDLLVVTSFNLWGQGTLVTVSS

SEQ ID NO: 53 - protein, 123 aa, Oryctolagus cuniculus
RSLEESGGDLVKPGTSLTLTCTASGFSFSGNYYMCWVRQAPGKGLEWIACIVVGSGGNTYYAGWAKRRFTISKTSSTTVTLQMTSLTAADTATYFCASGSYDDYGDYWYFTLWGQGTLVTVSS

SEQ ID NO: 54 - protein, 114 aa, Oryctolagus cuniculus
QSVEESGGRLVTPGGSLTLTCKVSGFSLSDYDIYWVRQAPGKGLEWIGVIDIENSVYYPTWAKGRFTISKTSTTVDLKITSPTTEDTATYFCARGDYIMTLDLWGQGTLVTVSS

SEQ ID NO: 55 - protein, 113 aa, Oryctolagus cuniculus
QSLEESGGDLVKPGASLTLTCTASGFSLSSGGMTWVRQAPGKGLEWIGYINTGSGRTYYASWAKGRFIISKTSSTTVSLQMTSLTAADTATYFCAGGLPSDLWGPGTLVTVSS

SEQ ID NO: 56 - protein, 117 aa, Oryctolagus cuniculus
QSVEESGGRLVTPGTPLTLTCTVSGFSLSSYGMIWVRQAPGEGLEWIGFIGRGGATWYASWVKGRFTISKTSTTVDLKITSPTASDTATYFCARDGDSSDYYAFNLWGQGTLVTVSS

SEQ ID NO: 57 - protein, 117 aa, Oryctolagus cuniculus
QSLEESGGRLVTPGTPLTLTCTVSGFSLSRCAMIWVRQAPGKGLEWIGFIGRGGSTWYASWVNGRFTISKTSTTVDLKITSPTTEDTATYFCARDGDYSDYYTFDLWGQGTLVTVSS

SEQ ID NO: 58 - protein, 117 aa, Oryctolagus cuniculus
QSLEESGGRLVTPGTPLTLTCTVSGFSLSSCAMIWVRQAPGKGLEWIGFIGRGGSTWYASWVNGRFTISKTSTTVDLKITSPTTEDTATYFCARDGDFSDYYTFNLWGQGTLVTVSS

SEQ ID NO: 59 - protein, 120 aa, Oryctolagus cuniculus
QSVEESGGRLVKPDETLTLTCTVSGIDLSSYAMGWVRQAPGKGLEYIGIISSSGRTYYANWAKGRFTISKASSTTVDLKITSPTTEDTATYFCARLITVDYYIYDYFNLWGQGTLVTVSS

SEQ ID NO: 60 - protein, 112 aa, Oryctolagus cuniculus
QSLEESGGRLVTPGTPLTLTCKASGFSLSSYWMSWVRQARGKGLEWIGMIYGSGYTYYASWAKGRFTISTTSTTVDLSVTSPTAEDTATYFCARDPQYFILWGQGTQVTVSS

SEQ ID NO: 61 - protein, 119 aa, Oryctolagus cuniculus
QSVEESGGRLVTPGGSLTLTCTVSGFSLSIYAMGWFRQAPGKGLEWIGDIYAGSGSTWYASWVKGRFTISSTSTTVDLKITSPTTEDTATYFCAREIDAGYVGYGFNLWGQGTLVTVSS

SEQ ID NO: 62 - protein, 123 aa, Oryctolagus cuniculus
QQLEQSGGGAEGGLVKPGGSLELCCKASGFSLSTSYWRCWVRQAPGKGLEWIGCIYAGSGDVTYYANWVNGRFTLSRDIDQSTGCLQLNSLTAADTAMYYCASGVGFGYFNLWGQGTLVTVSS

SEQ ID NO: 63 - protein, 113 aa, Oryctolagus cuniculus
QSVEESGGRLVTPGTPLTLTCTVSGIDLSSYDMSWVRQAPGEGLEWIGTIYVSGRVYYATWAKGRFTISKTSSTTVDLEITSPTTEDTATYFCARGSIDYDPWGPGTLVTVSS

SEQ ID NO: 64 - protein, 116 aa, Oryctolagus cuniculus
QSLEESGGGLVQPEGSLTLTCTASGFSFSSSYWICWVRQAPGKGLEWIGCIYTGSGGTYYASWEKGRFTISKTSSTTVTLQMTSLTAADTATYFCARDPGYSSWLWGQGTLVTVSS

SEQ ID NO: 65 - protein, 121 aa, Oryctolagus cuniculus
QSVEESGGRLVTPGTPLTLTCTVSGIDLSTYTMSWVRQAPGKGLEYIGIILSSGSTYYATWAKGRFTISKTSSTTVDLKMTSLTTEDTAMYFCARGGPGYSIDTKYAFDPWGPGTLVTVSS

SEQ ID NO: 66 - protein, 119 aa, Oryctolagus cuniculus
QEQLEESGGGLVQPEGSLTLTCTASGFSFSSGYDMCWVRQAPGKGLEWIGCIYTGSGSTYYANWAKGRFTISKTSSTTVTLQMTSLTAADTATYFCARNSNDWMYFNLWGPGTLVTVSS

SEQ ID NO: 67 - protein, 120 aa, Oryctolagus cuniculus
QSLEESGGRLVTPGTPLTLTCTASGFTISSYHMSWVRQAPGKGLEWIGGIATDGNTYYANWAKGRFTVSRTSTTVDLKVTSPTAEDTATYFCARGGPAYSRGTHYAMDLWGPGTLVTVSS

SEQ ID NO: 68 - protein, 117 aa, Oryctolagus cuniculus
QSLEESGGRLVTPGTPLTLTCTVSGIDLSSYAMSWVRQAPGKGLEYIGVIGSSGNLYYASWAKGRFTISKTSTTVDLKMTSLTTEDTATYFCARYTIDSGIYTYDLWGQGTLVTVSA

SEQ ID NO: 69 - protein, 117 aa, Oryctolagus cuniculus
QSVEESGGRLVTPGTPLTLTCTVSGFDSSVYAMSWVRQAPGKGLEWIGISVSNIRTWYATWAKGRFTISKTSTMVDLKMTSLTTEDTATYFCARHVSRSGNYGLDLWGQGTLVTVSS

SEQ ID NO: 70 - protein, 119 aa, Oryctolagus cuniculus
QSVEESGGRLVTPGGSLTLTCTVSGFSLSVYAMGWFRQAPGKGLEWIGDIYAGSVNTWYATWAKGRFTISKTSTTVDLKITSPTTEDTATYFCAREIDAGYVGYGFNLWGQGTLVTVSS

SEQ ID NO: 71 - protein, 112 aa, Oryctolagus cuniculus
QSLEESGGRLITPGGSLTLTCTVSGFSLSSYHMQWVRQAPGKGLEYIGYINSLGGSYYASWAKGRFTISKTSTTVDLKITSPTTADTATYFCARDFAGSDLWGQGTLVTVAS

SEQ ID NO: 72 - protein, 119 aa, Oryctolagus cuniculus
QSVEESGGRLVTPGTPLTLTCTVSGFSLNDYAMIWVRQAPGEGLEYIGFIEPGGRAYCASWAKGRFTISRTSTTVDLKMTSLTTEDTATYFCARSYVFYSTYPYASDLWGQGTLVTVSS

SEQ ID NO: 73 - protein, 121 aa, Oryctolagus cuniculus
QEQLVESGGGLVQPEGSLTLTCTASGFSFSSIYYMCWVRQAPGKGLEWIGCIYTGNSDFTYYANWAKGRLSISRSTSLSTVTLQMTSLTAADTATYFCARFRDDYASLKLWGPGTLVTVSS

SEQ ID NO: 74 - protein, 118 aa, Oryctolagus cuniculus
QEHLMESGGGLVQPEGSLTLSCTASGFSFSSTYWICWVRQAPGKGLEWIGCINTGSGGSTYYANWVKGRFTISKTSSTTVTLQMTSLTAADTATYFCARGDDSYYELWGQGTLVTVSS

SEQ ID NO: 75 - protein, 126 aa, Oryctolagus cuniculus
QQLEQSGGGAEGGLVKPGGSLELCCKASGFSQSNNYWMHWVRQAPGKGLEWIGCIYAGSSDSTYYASWVNGRFTLSRDIDQSIGCLQLNSLTAADTAIYYCARAIADFSSGWGDLWGQGTLVTVSS

SEQ ID NO: 76 - protein, 111 aa, Oryctolagus cuniculus
QSLEESGGGLVTPGASLTLTCTASGFTLSSDYWICWVRQAPGKGLEWIACIYAGSSVTYYARWAKGRFTISKTSSTTVTLQMTSLTAADTATYFCARGGLWGPGTLVTVSS

SEQ ID NO: 77 - protein, 118 aa, Oryctolagus cuniculus
QSLEESGGRLVTPGTPLTLTCTVSGFSLSRCAMIWVRQAPGKGLEWIGFIGRGGSTWYASWVNGRFTISKTSSTTVDLKITSPTTEDTATYFCARDGDYSDYYTFDLWGQGTLVTVSS

SEQ ID NO: 78 - protein, 110 aa, Oryctolagus cuniculus
AFEMTQTPSSVSEPVGGTVTIKCQASQSIYIYLSWYQQKPGQRPKLLIYDASKLASGVPSRFSGSGSGTEFTLTISGVQSDDAATYYCQQGATTYNVDNVFGGGTEVVVK

SEQ ID NO: 79 - protein, 110 aa, Oryctolagus cuniculus
ALVMTQTPASVEAAVGGTVTIKCQASQTISINLAWYQQKPGQRPKLLIYYASTLASGVPSRFSGSGSGTEFTLTISGVQSDDAATYYCQQGYTEDNIDNTFGGGTEVVVK

SEQ ID NO: 80 - protein, 111 aa, Oryctolagus cuniculus
ALVMTQTPSSVSAAVGGTVTINCQASENIYSSLAWYQQKPGQPPKLLIYDASDLASGVPSRFKGSGSGKEFTLTISDLESDDAATYYCQQGYYSGGTDNDVFGGGTEVVVK

SEQ ID NO: 81 - protein, 108 aa, Oryctolagus cuniculus
DVVMTQTPASVSEPVGGTVTIKCQASQSIYSFLSWYQQKPGQPPKLLIYAASDLESGVPSRFSGSGYGTEFTLTISDLESADAATYYCQCNYIIDYGAFGGGTEVVVK

SEQ ID NO: 82 - protein, 110 aa, Oryctolagus cuniculus
ALVMTQTPSPVSAAVGGTVTINCQASEDIYSNLAWFQQKPGQPPKLLIYRASTLASGVPSRFKGSGSGTEFTLTISGLQSDDAATYYCLGVYTYPSADNAFGGGTEVVVR

SEQ ID NO: 83 - protein, 111 aa, Oryctolagus cuniculus
ALVMTQTPASVSEPVGGTVTIKCQASENIGNGLAWYQQKPGQPPNLLIYGASTLASGVPSRFSGSGYGTEFTLTVSDLESGDAATYYCQCTYWNPDYIGGAFGGGTEVVVT

SEQ ID NO: 84 - protein, 110 aa, Oryctolagus cuniculus
AIEMTQSPPSLSASVGETVRIRCLASEDIYSGISWYQQKPGKPPTLLIYAASNLESGVPPRFSGSGSGTDYTLTIGGVQAEDAATYYCLGGYSYSNTGPTFGAGTKVEIK

SEQ ID NO: 85 - protein, 110 aa, Oryctolagus cuniculus
AIEMTQTPFSVSAAVGGTVTINCQASESIYSNLAWYQQILGQPPKLLIYAASLLASGVPSRFKGSGSGTEYTLTISGVQSADAATYYCQSASYSTGPDWTFGGGTEVVVE

SEQ ID NO: 86 - protein, 110 aa, Oryctolagus cuniculus
AFEMTQTPSSVSEPVGGTVTIKCQASQSIYNYLSWYQQKPGQRPKLLIYRASTLASGVPSRFKGSGSGTEFTLTISGVESADAATYYCQQGATSYDIENPFGGGTEVVVK

SEQ ID NO: 87 - protein, 110 aa, Oryctolagus cuniculus
AFEMTQTPSSVSEPVGGTVTIKCQASQSIYNYLSWYQQKPGQPPKLLIYDASELASGVPSRFKGSGSGTEFTLTISGVQSDDAATYYCQQGATTYNIENVFGGGTEVVVK

SEQ ID NO: 88 - protein, 111 aa, Oryctolagus cuniculus
AQALTQTPSSVSAAVGGTVTINCQSSQSVYSDYLAWYQQKPGQPPKLLIYQASKLATGVPSRFKGSGSGTQFTLTISGVQSDDAATYYCQATYYGSGWYRAFGGGTELVVK

SEQ ID NO: 89 - protein, 111 aa, Oryctolagus cuniculus
ALVMTQTPSPVSAAVGGTVTINCQASEDIYSNLAWFQQKPGQPPKLLIYRASTLASGVPSRFSGSGSGTEFTLTISALQSDDAATYYCLGVRTYFNTLNNSFGGGTEVVVK

SEQ ID NO: 90 - protein, 111 aa, Oryctolagus cuniculus
AQALTQTPSSVSAAVGGTVTINCQSSQSVYSDYLAWYQQKPGQSPKLLIYKASKLASGVPSRFKGSGSGTEFTLTISGVQSDDAATYYCQATYYSVGWYRAFGGGTEVVVK

SEQ ID NO: 91 - protein, 111 aa, Oryctolagus cuniculus
ALVLTQTPSPVSAAVGGTVTINCQASEDIYSNLAWFQQKPGQPPKLLIYRASTLASGVPSRFSGSGSGTEFTLTISGLQSDDAATYYCLGVYTYLSDLFFVFGGGTEVVVK

SEQ ID NO: 92 - protein, 110 aa, Oryctolagus cuniculus
ALVMTQTPSPVSAAVGGTVTINCQASEDIYSNLAWFQQKPGQPPKLLIYRASTLASGVPSRFSGSGSGTEFTLTISGLQSDDAATYYCLGVYTYSTDIHAFGGGTEVVVK

SEQ ID NO: 93 - protein, 109 aa, Oryctolagus cuniculus
DVVMTQTPASVSEPVGGTVTINCQASESISDYLSWYQQKPGQPPKLLIYRASTLESGVSSRFKGSGSGTQFTLTISDLESADAATYYCQSNYYDSRGNAFGGGTEVVVK

SEQ ID NO: 94 - protein, 110 aa, Oryctolagus cuniculus
AQVLTQTASSVSATVGGTVTISCQSSQSVYNNNYLSWYQQKPGQPPKLLIYKASTLASGVPLRFSGSGSGTQFTLTISGVQSDDAATYYCAGFYETTDVGFGGGTEVVVK

SEQ ID NO: 95 - protein, 113 aa, Oryctolagus cuniculus
AQVLTQTPSPVSAAVGGTVTISCQASQSVYNSNHLSWYQQKPGQPPRLLIYSASTLASGVPSRFKGSGSGTQFTLTISGVQSDDAATYYCQGEFSCVSADCIAFGGGTEVVVK

SEQ ID NO: 96 - protein, 110 aa, Oryctolagus cuniculus
AIEMTQTPSSVSAAVGGTVTINCQASQNIYSNLAWYQQKPGQRPKLLIYAASLLASGVPSRFKGNGSGTEYTLTISDLESADAATYYCQGAVYSGNTEWAFGGGTEVVVK

SEQ ID NO: 97 - protein, 113 aa, Oryctolagus cuniculus
ALMMTQTPSPVSAAVGGTVTINCQASEDIYSNLAWYQQKPGQPPKLLIYSASTLASGVPSRFSGSGSGTEFTLTISGVQSDDAATYYCLGVCTDISVDDVYNSFGGGTEVVVK

SEQ ID NO: 98 - protein, 114 aa, Oryctolagus cuniculus
ALVMTQTPSPVSAAVGGTVTINCQASEDIYSNLAWFQQKPGQPPKLLIYQASTLASGVPSRFSGSGSGTEFTLTISGLQSDDAAAYYCLGVCTYIGADNTLYNTFGGGTEVVVK

SEQ ID NO: 99 - protein, 110 aa, Oryctolagus cuniculus
ALVMTQTPSPVSAAVGGTVTINCQASEDIYSNLAWFQQKPGQPPKLLIYDASTLASGVPSRFSGSGSGTEFTLTISGLQSDDAATYYCLGVYTHISADNAFGGGTEVVVK

SEQ ID NO: 100 - protein, 110 aa, Oryctolagus cuniculus
AFEMTQTPSSVSEPVGGTVTIKCQASQSIHNYLSWYQQKPGQRPKLLIYRASTLASGVPSRFKGSGSGTEFTLTISGVESADAATYYCQQGATSYDIDNAFGGGTEVVVK

SEQ ID NO: 101 - protein, 110 aa, Oryctolagus cuniculus
AFEMTQTPASVEVAVGGTVTINCQASQSIGSWLSWYQQKVGQRPKLLISRASTLASGVPSRFKGSGSGTEYTLTISGVQSDDAATFYCQQGATTYDVDNVFGGGTEVVVR

SEQ ID NO: 102 - protein, 110 aa, Oryctolagus cuniculus
AYDMTQTPASVEAAVGGTVNIKCQASQSISNWLAWYQQKPGQRPKLLIYRASTLASGVSSRFKGSGSGTQFTLTISGVESADAATYYCQQGASTTDVDNVFGGGTEVVVK

SEQ ID NO: 103 - protein, 108 aa, Oryctolagus cuniculus
DIVMTQTPASVEAAVGGTVTIKCQASQSIGYYLAWYQQKPGQPPKLLISRASTLASGVPSRFKGSGSGTQFTLTISDLESADVATYYCQSYYNSDSDAFGGGTEVVVK

SEQ ID NO: 104 - protein, 111 aa, Oryctolagus cuniculus
AQALTQTPSPVSAAVGGTVTINCQSSQSVYSDYLGWYQQKPGQPPKLLIYWASKLETGVPSRFKGSGSGTQFTLTISGVQSDDAATYYCQATYYGSGWYRAFGGGTEVVVK

SEQ ID NO: 105 - protein, 113 aa, Oryctolagus cuniculus
ALVMTQTPSPVSAAVGGTVTISCQASEDIYSNLAWYQQKRGQPPKLLIYYASTLASGVPSRFSGSGSGTEFTLTISGLQSDDAATYYCLGVCTYINANGWDNAFGGGTEVVVK

SEQ ID NO: 106 - protein, 110 aa, Oryctolagus cuniculus
AYDMTQTPASVEVAVGGTVTIKCQASQSIYIYLAWYQQKPGQRPKQLIYDASKLASGVPSRFSGSGSGTEFTLTISGVESADAATYYCQQGATIWNVDNPFGGGTEVVVK

SEQ ID NO: 107 - protein, 110 aa, Oryctolagus cuniculus
AYDMTQTPASVEAAVGGTVNIKCQASQSISNWLAWYQQKPGQPPKLLIYRASTLASGVSSRFKGSGSGTQFTLTISGVESADAATYYCQQGASTTDVDNVFGGGTEVVVK

SEQ ID NO: 108 - protein, 110 aa, Oryctolagus cuniculus
DIVMTQTPSPVSGAVGGTVTIKCQASEDIYSNLAWYQQKPGQPPKLLIYYVSTLESGVPSRFKGSRSGTDYTLTISDLESADAATYYCQCTEGGSGSDYTFGGGTEVVVK

SEQ ID NO: 109 - protein, 114 aa, Oryctolagus cuniculus
ALVMTQTPSPVSAAVGGTVTINCQASEDIYSNLAWFQQKPGQPPKLLIYRASTLASGVPSRFSGSGSGTEFTLTISGVQSDDAATYYCLGIYTYISADGSLYNAFGGGTEVVVK

SEQ ID NO: 110 - protein, 114 aa, Oryctolagus cuniculus
ALVMTQTPSPVSAAVGGTVTINCQASEDIYSNLAWFQQKPGQPPKLLIYQASKLASGVPSRFSGSGSGTEFTLTISGLQSDDVATYYCLGVGTYISGDGSLDNAFGGGTEVVVK

SEQ ID NO: 111 - protein, 110 aa, Oryctolagus cuniculus
AYDMTQTPASVEAAVGGTVTIKCQASQSISNWLAWYQQKPGQRPKLLIYRASTLASGVSSRFKGSGSGTQFTLTISGVESADAATYYCQQGASTTDVDNVFGGGTEVVVK

SEQ ID NO: 112 - protein, 111 aa, Oryctolagus cuniculus
DVVMTQTPASVEAAVGGTVTIKCQASQNIYSNCAWYQQKLGQRPKLLIYYVSTLESGVPSRFEGSGYGTEFTLTISDLQSADAATYYCQYTYDSSSSTSWAFGGGTEVVVK

SEQ ID NO: 113 - protein, 112 aa, Oryctolagus cuniculus
DIVMTQTPASVEAAVGGTITINCQASENIYSSLAWYQQKPGQPPKLLIYDASTLASGVSSRFKGSGSGTQFTLTISGVQSDDAATYYCQSYYCSVSSSCGYGFGGGTEVVVK

SEQ ID NO: 114 - protein, 115 aa, Oryctolagus cuniculus
ALVMTQTPSPVSAAVGGTVTINCQASEDIYSNLAWFQQKPGQPPKLLIYDASTLASGVPSRFSGSGSGTEFTLTISGLQSDDAATYYCLGVYTYISADGTLVYNAFGGGTEVVVR

SEQ ID NO: 115 - protein, 113 aa, Oryctolagus cuniculus
ALVMTQTPSPVSAAVGGTVTINCQASEDIYSNLAWFQQKPGQPPKLLIYGASTLASGVPSRFSGSGSGTEFTLTISGVQSDDAATYYCLGVCTDISTDDLYNAFGGGTELVVK

SEQ ID NO: 116 - protein, 111 aa, Oryctolagus cuniculus
AQALTQTPSSVSAAVGGTVTINCQSSQSVYSDYLVWYQQKPGQPPKLLIYQASKLASGVPSRFKGSGSGTQFTLTISGVQSDDAATYYCQATYSSTGWYRAFGGGTEVVVK

SEQ ID NO: 117 - protein, 112 aa, Oryctolagus cuniculus
DIVMTQTPASVSAPVGGTVTINCQASESIYSDLAWYQQKPGQPPKLLISFVATLESGVPSRFKGSGSGTEFTLTISDLESADAATYYCQCTYGGSGSGNGAAFGGGTEVVVK

SEQ ID NO: 118 - protein, 111 aa, Oryctolagus cuniculus
ALVMTQTPSPVSAAVGGTVTINCQASEDIYSNLAWYQQKPGQPPKLLMYGVSTLASGVPSRFSDSGSGTEFTLTISGLQSDDAATYYCLGVYTYISDVYYTFGGGTEVVVK

SEQ ID NO: 119 - protein, 109 aa, Oryctolagus cuniculus
DVVMTQTPASVEAAVGGTVTIKCQASQSISSYCSWYQQKPGQPPKLLIYRASTLESGVPSRFKGSGSGTEFTLTISDLESADAATYYCQSSYYDLLGNGFGGGTEVVVK

SEQ ID NO: 120 - protein, 113 aa, Oryctolagus cuniculus
ALVMTQTPSPVSAAVGGTVTINCQASEDIYSNLAWFQQKPGQPPKLLIYDASTLASGVPSRFSGSGSGTEFTLTISGLQSDDAATYYCLGVYTYISPDGTDNAFGGGTEVVVK

SEQ ID NO: 121 - protein, 111 aa, Oryctolagus cuniculus
ALVMTQTPSPVSAAVGGTVTINCQASQNIASAYLSWYQQKPGQPPKLLIYAASTLTDGVPSRFKGSGSVTEFTLTISGVQSDDAATYYCAGYKSYTDDEFAFGGGTEVVVK

SEQ ID NO: 122 - protein, 110 aa, Oryctolagus cuniculus
AYDMTQTPASVEVAVGGTVTIKCQASESISTWLAWYQQKPGQPPNLLIYRASTLASGVPSRFYGSGYGTEFTLTISGVESADAATYYCQQGYTVNNIDNVFGGGTEVVVK

SEQ ID NO: 123 - protein, 110 aa, Oryctolagus cuniculus
DIVMTQTPASVEAAVGGTVTIRCQASQSISSYLAWYQRKPGQPPKVLIYKASTLASGVSSRFKGSGSGTEYTLTISDLESADAATYYCQQGYASSGVDNVFGGGTEVVVK

SEQ ID NO: 124 - protein, 113 aa, Oryctolagus cuniculus
DIVMTQTPASVSEPVGGTVTIRCQASQSISSWLSWYQQKPGQPPKLLIYQASALASGVSSRFIGSGYGTEFTLTISGVQSEDAATYYCQCTYGIGSNSDYGVAFGGGTEVVVK

SEQ ID NO: 125 - protein, 113 aa, Oryctolagus cuniculus
DIVMTQTPASVSEPVGGTVTIKCQASQSITTWLAWYQQKPGQPPKLLIYQASALASGVSSRFIGSGYGTEFTLTISGVQSEDAATYYCQCTYGIGSGSSYGVAFGGGTEVVVK

SEQ ID NO: 126 - protein, 113 aa, Oryctolagus cuniculus
DVVMTQTPASVEAAVGGTVTIMCQASETIYSGLAWYQQKPGQPPKLLIYYTSSLASGVPSRFKGSGSGTEFTLTISDLESADAATYYCQTYYDSEGRSYGYNSFGGGTEVVVK

SEQ ID NO: 127 - protein, 112 aa, Oryctolagus cuniculus
DIVMTQTPSSVEAAVGGTVTIKCQASQSINSRLAWYQQKPGQPPKLLIYSASTLASGVSSRFKGSGSGTEFTLTISDLESADGATYYCLSHYLTSSSSYGDAFGGGTEVVVK

SEQ ID NO: 128 - protein, 111 aa, Oryctolagus cuniculus
AQVLTQTPSSVSAAVGGTVTINCQSSPSVYNNYLSWYQQKPGQPPKLLIYGASSLASGVPSRFKGSGSGTQFTLTISDLESDDAATYYCQGGYNSYSDTFAFGGGTEVLVK

SEQ ID NO: 129 - protein, 113 aa, Oryctolagus cuniculus
ALVMTQTPSPVSAAVGGTVTINCQASEDIYSNLAWFQQKPGQPPKLLIYGASTLASGVPSRFSGSGSGTEFTLTISGVQSDDAATYYCLGVCTDISADDLYNTFGGGTEVVVK

SEQ ID NO: 130 - protein, 110 aa, Oryctolagus cuniculus
AIDMTQTPSPASAGVGDTVTINCQASENIYNFLAWYQQKPGHSPKLLIYVASKLASGVPSRFKGSGSGTQFTLTISDVQSDDAATYYCQQTYRYNDGDTAFGGGTEVVVK

SEQ ID NO: 131 - protein, 111 aa, Oryctolagus cuniculus
AAVLTQTPSPVSAAVGGTVTISCQASQSVYKNNRLAWYQQKPGQPPKLLIYLASTLASGVPSRFKGSGSGTQFTLTISDLESDDAATYYCAGGYSTISENAFGGGTEVVVK

SEQ ID NO: 132 - protein, 110 aa, Oryctolagus cuniculus
DIVMTQTPSSVEAAVGGTVTIKCQASQSINSRLAWYQQKPGQPPKLLIYDASTLASGVSSRFSGSGTEFTLTISDLESADGATYYCLSHYLTSSSSYGNAFGGGTEVVVK

SEQ ID NO: 133 - protein, 110 aa, Oryctolagus cuniculus
DVVMTQTPASVSEPVGGTVTIKCQASQNIGSNLAWYQQRSGQPPKLLIYGASTLASGVPSRFSGSGSGTEFTLTISGVQSADAATYFCQCSGYDITGVFPFGGGSEVVVK

SEQ ID NO: 134 - protein, 110 aa, Oryctolagus cuniculus
DVVMTQTPASVSERVGGTVTIKCQASQSIGSNLAWYQQKPGQPPKLLIYGASNLESGVPSRFSGSGSGTEFTLSISGVQSADAATYYCQCSGYDTTGVFPFGGGSEVVVK

SEQ ID NO: 135 - protein, 110 aa, Oryctolagus cuniculus
DVVMTQTPASVSEPVGGTVTIKCQASQNIGSNLAWYQQKPGQPPKLLIYGASTLASGVPSRFSGSGSGTEFTLTISGVQSADATTYYCQCSGYDTTGVFPFGGGSEVVVR

SEQ ID NO: 136 - protein, 110 aa, Oryctolagus cuniculus
AYDMTQTPASVEAAVGGTVTIKCQASQSISSYLSWYQQKPGQPPKLLIYGASTLASGVPSRFKGSGSGTEYTLTISGVESDDAATYYCQQGYSYNNVDNTFGGGTEVVVK

SEQ ID NO: 137 - protein, 112 aa, Oryctolagus cuniculus
QAVVTQTPSPVSAAVGGTVIISCQSSQSVDGNNLLSWYQQKPGQPPKLLIYDASNLASGVPSRFSGSGSGTQFTLTISGVQSDDAATYYCQGSYYSSSWYNVFGGGTEVVVK

SEQ ID NO: 138 - protein, 111 aa, Oryctolagus cuniculus
AQALTQTPSSVSAAVGGTVTINCQSSQSVYSDYLAWYQQKPGQPPKLLIYWASKLASGVPSRFKGSGSGTQFTLTISGVQSDDAATYYCQATYNGRGWYRAFGEGTEVVVK

SEQ ID NO: 139 - protein, 110 aa, Oryctolagus cuniculus; identical to SEQ ID NO: 99

SEQ ID NO: 140 - protein, 111 aa, Oryctolagus cuniculus
AYDMTQTPASVEAAVGGTVTIKCQASQSISSWLSWYQQKPGQPPKQLIYRASTLASGVSSRFKGSGSGTDYTLTISGVQSDDAATYYCQQGYITSSNIKNVFGGGTEVVVK

SEQ ID NO: 141 - protein, 110 aa, Oryctolagus cuniculus
DVVMTQTPASVSGPVGGTVTINCQASESISNYLSWYQQKSGQPPKLLIYLASTLASGVPSRFKGSGSGTEFTLTISDLESADAATYYCQNWWVIEHNGAAFGGGTEVVVK

SEQ ID NO: 142 - protein, 110 aa, Oryctolagus cuniculus
AFEMTQTPSSVSEPVGGTVTIKCQASQNIYIYLSWYQQKPGQPPKLLIYDASTLASGVSSRFSGSGSGTEFTLTISGVQSEDAAIYYCQQGATTYDVDNVFGGGTEVVVK

SEQ ID NO: 143 - protein, 114 aa, Oryctolagus cuniculus
DIVMTQTPASVEAAVGGTVTIKCQASESISANYWSWYQQKPGQPPKLLIYGASTLASGVPSRFKGSGSGPQFTLTISDLESADAATYFCQSWYYSGSGSYHSWAFGGGTEVVLK

SEQ ID NO: 144 - protein, 110 aa, Oryctolagus cuniculus; identical to SEQ ID NO: 106

SEQ ID NO: 145 - protein, 111 aa, Oryctolagus cuniculus
AAVLTQTPSPVSAAVGGTVSISCQSSQSVYGNNELSWFQQKPGQPPKLLIYGASILASGVPSRFSGSGSGTEFTLTISDVQSDDAATYYCAGGYSSTSDNAFGGGTEVVVK

SEQ ID NO: 146 - protein, 111 aa, Oryctolagus cuniculus
DVVMTQTPASVSEPVGGTVTIKCQASEDISSYLAWYQQKPGQPPKLLIYDASDLASGVPSRFSGGGYGTEFSLTISDLESADAATYYCQCADYATTYGLGAFGGGTEVVVK

SEQ ID NO: 147 - protein, 111 aa, Oryctolagus cuniculus
AQALTQTPSSVSAAVGGTVTINCQSSQSVYSDYLAWYQQKPGQPPKLLISQASKLASGVPSRFKGSGSGTQFTLTISDLESDDAATYYCQATYSSSGWYRAFGGGTEVVVK

SEQ ID NO: 148 - protein, 110 aa, Oryctolagus cuniculus
AYDMTQTPASVEVAVGGTVTIKCQASQSIDYYLAWYQQKPGQPPKLLIYRASTLASGVSSRFKGSGSGTDYTLTISGVESADAATYYCQQGYSGNNVDNTFGGGTEVVVK

SEQ ID NO: 149 - protein, 112 aa, Oryctolagus cuniculus
AQVLTQTASPVSAAVGGTVTINCQSSQSVNGNNYLAWYQQKPGQPPKLLIWLASSLASGVPSRFKGSGSGTQFALTISDLESDDAATYYCAGAYSTSGEENAFGGGTEVVVK

SEQ ID NO: 150 - protein, 110 aa, Oryctolagus cuniculus
DVVMTQTPASVSEPVGGTVTIKCQASQSISSYLSWYQQKPGQPPKLLIYGASNLASGVPSRFKGSGSGTEFTLTISDLESADAATYYCQCTYYDNNYGGAFGGGTEVVVK

SEQ ID NO: 151 - protein, 110 aa, Oryctolagus cuniculus
DTVLTQTPSSVSAAVGDTVTIKCQASQNIYSGLAWYQQKPGQPPKLLIYYASTLASGVPSRFKGSGSGTEFTLTISDLESADAATYYCQTYYGVYVYGIIFGGGTEVVVK

SEQ ID NO: 152 - protein, 111 aa, Oryctolagus cuniculus
ALVMTQTPSPVSAAVGGTVTINCQASEDIYSNLAWFQQKPGQPPKLLIYGASTLASGVPSRFSGSGSGTEFTLTISGLQSDDAATYYCLGVCTDISAVYNVFGGGTEVVVK

SEQ ID NO: 153 - protein, 113 aa, Oryctolagus cuniculus
AQVLTQTPSSMSAAVGGTVTINCQASQSVYKNNYLSWYQQKPGQPPKRLMYSASTLDSGVPLRFSGSGSGTQFTLTISDVQSEDAATYYCQGNYDCSSADCIAFGGGTEVVVK

SEQ ID NO: 154 - protein, 110 aa, Oryctolagus cuniculus
DVVMTQTPASVSEPVGGTVTIKCQASQNIGSNLAWYQQKPGQPPKLLIYGASTLASGVPSRFSGSGSGTEFTLTISGVQSADAATYYCQCSGYDTTGVFPFGGGSEVVVK

SEQ ID NO: 155 - protein, 5 aa, Oryctolagus cuniculus
SYAMG

SEQ ID NO: 156 - protein, 6 aa, Oryctolagus cuniculus
FGYYMC

SEQ ID NO: 157 - protein, 6 aa, Oryctolagus cuniculus
SYYYMC

SEQ ID NO: 158 - protein, 5 aa, Oryctolagus cuniculus
SYDMS

SEQ ID NO: 159 - protein, 6 aa, Oryctolagus cuniculus
SSYWIC

SEQ ID NO: 160 - protein, 5 aa, Oryctolagus cuniculus
NYAMG

SEQ ID NO: 161 - protein, 5 aa, Oryctolagus cuniculus
SYYMS

SEQ ID NO: 162 - protein, 6 aa, Oryctolagus cuniculus
SSHYMC

SEQ ID NO: 163 - protein, 5 aa, Oryctolagus cuniculus
NYAMS

SEQ ID NO: 164 - protein, 5 aa, Oryctolagus cuniculus
NYAMS

SEQ ID NO: 165 - protein, 5 aa, Oryctolagus cuniculus
IYAMG

SEQ ID NO: 166 - protein, 6 aa, Oryctolagus cuniculus
TNYWIC

SEQ ID NO: 167 - protein, 5 aa, Oryctolagus cuniculus
IYAMG

SEQ ID NO: 168 - protein, 6 aa, Oryctolagus cuniculus
SDAWIC

SEQ ID NO: 169 - protein, 6 aa, Oryctolagus cuniculus
SSYYMC

SEQ ID NO: 170 - protein, 5 aa, Oryctolagus cuniculus
NYHIS

SEQ ID NO: 171 - protein, 5 aa, Oryctolagus cuniculus
SNGIN

SEQ ID NO: 172 - protein, 6 aa, Oryctolagus cuniculus
SSDFMC

SEQ ID NO: 173 - protein, 6 aa, Oryctolagus cuniculus
STYYMC

SEQ ID NO: 174 - protein, 6 aa, Oryctolagus cuniculus
SNYWMC

SEQ ID NO: 175 - protein, 6 aa, Oryctolagus cuniculus
SSYWIC

SEQ ID NO: 176 - protein, 6 aa, Oryctolagus cuniculus
TSYWRC

SEQ ID NO: 177 - protein, 5 aa, Oryctolagus cuniculus
NYAMS

SEQ ID NO: 178 - protein, 5 aa, Oryctolagus cuniculus
SDAVG

SEQ ID NO: 179 - protein, 5 aa, Oryctolagus cuniculus
SYYMS

SEQ ID NO: 180 - protein, 6 aa, Oryctolagus cuniculus
SNYYMC

SEQ ID NO: 181 - protein, 5 aa, Oryctolagus cuniculus
VYAMG

SEQ ID NO: 182 - protein, 6 aa, Oryctolagus cuniculus
SAYWIC

SEQ ID NO: 183 - protein, 5 aa, Oryctolagus cuniculus
SYYMS

SEQ ID NO: 184 - protein, 5 aa, Oryctolagus cuniculus
SYYMS

SEQ ID NO: 185 - protein, 5 aa, Oryctolagus cuniculus
SYAMG

SEQ ID NO: 186 - protein, 6 aa, Oryctolagus cuniculus
NSYWIC

SEQ ID NO: 187 - protein, 6 aa, Oryctolagus cuniculus
SSYWIC

SEQ ID NO: 188 - protein, 5 aa, Oryctolagus cuniculus
SYYMS

SEQ ID NO: 189 - protein, 6 aa, Oryctolagus cuniculus
SYYVIC

SEQ ID NO: 190 - protein, 5 aa, Oryctolagus cuniculus
GYAMS

SEQ ID NO: 191 - protein, 6 aa, Oryctolagus cuniculus
SSYWIC

SEQ ID NO: 192 - protein, 6 aa, Oryctolagus cuniculus
SNYWIC

SEQ ID NO: 193 - protein, 5 aa, Oryctolagus cuniculus
VYAMG

SEQ ID NO: 194 - protein, 5 aa, Oryctolagus cuniculus
SYAMG

SEQ ID NO: 195 - protein, 6 aa, Oryctolagus cuniculus
SSYWIC

SEQ ID NO: 196 - protein, 5 aa, Oryctolagus cuniculus
TYYMS

SEQ ID NO: 197 - protein, 6 aa, Oryctolagus cuniculus
SNYWMC

SEQ ID NO: 198 - protein, 6 aa, Oryctolagus cuniculus
SAYWMC

SEQ ID NO: 199 - protein, 6 aa, Oryctolagus cuniculus
ASYWIC

SEQ ID NO: 200 - protein, 6 aa, Oryctolagus cuniculus
SGYYMC

SEQ ID NO: 201 - protein, 5 aa, Oryctolagus cuniculus
IYGMG

SEQ ID NO: 202 - protein, 5 aa, Oryctolagus cuniculus
VYNMG

SEQ ID NO: 203 - protein, 5 aa, Oryctolagus cuniculus
DYYMG

SEQ ID NO: 204 - protein, 5 aa, Oryctolagus cuniculus
SGGMS

SEQ ID NO: 205 - protein, 6 aa, Oryctolagus cuniculus
YTYVMC

SEQ ID NO: 206 - protein, 6 aa, Oryctolagus cuniculus
SNYWIC

SEQ ID NO: 207 - protein, 6 aa, Oryctolagus cuniculus
GNYYMC

SEQ ID NO: 208 - protein, 5 aa, Oryctolagus cuniculus
DYDIY

SEQ ID NO: 209 - protein, 5 aa, Oryctolagus cuniculus
SGGMT

SEQ ID NO: 210 - protein, 5 aa, Oryctolagus cuniculus
SYGMI

SEQ ID NO: 211 - protein, 5 aa, Oryctolagus cuniculus
RCAMI

SEQ ID NO: 212 - protein, 5 aa, Oryctolagus cuniculus
SCAMI

SEQ ID NO: 213 - protein, 5 aa, Oryctolagus cuniculus
SYAMG

SEQ ID NO: 214 - protein, 5 aa, Oryctolagus cuniculus
SYWMS

SEQ ID NO: 215 - protein, 5 aa, Oryctolagus cuniculus
IYAMG

SEQ ID NO: 216 - protein, 6 aa, Oryctolagus cuniculus
TSYWRC

SEQ ID NO: 217 - protein, 5 aa, Oryctolagus cuniculus
SYDMS

SEQ ID NO: 218 - protein, 6 aa, Oryctolagus cuniculus
SSYWIC

SEQ ID NO: 219 - protein, 5 aa, Oryctolagus cuniculus
TYTMS

SEQ ID NO: 220 - protein, 6 aa, Oryctolagus cuniculus
SGYDMC

SEQ ID NO: 221 - protein, 5 aa, Oryctolagus cuniculus
SYHMS

SEQ ID NO: 222 - protein, 5 aa, Oryctolagus cuniculus
SYAMS

SEQ ID NO: 223 - protein, 5 aa, Oryctolagus cuniculus
VYAMS

SEQ ID NO: 224 - protein, 5 aa, Oryctolagus cuniculus
VYAMG

SEQ ID NO: 225 - protein, 5 aa, Oryctolagus cuniculus
SYHMQ

SEQ ID NO: 226 - protein, 5 aa, Oryctolagus cuniculus
DYAMI

SEQ ID NO: 227 - protein, 6 aa, Oryctolagus cuniculus
SIYYMC

SEQ ID NO: 228 - protein, 6 aa, Oryctolagus cuniculus
STYWIC

SEQ ID NO: 229 - protein, 6 aa, Oryctolagus cuniculus
NNYWMH

SEQ ID NO: 230 - protein, 6 aa, Oryctolagus cuniculus
SDYWIC

SEQ ID NO: 231 - protein, 5 aa, Oryctolagus cuniculus
RCAMI

SEQ ID NO: 232 - protein, 16 aa, Oryctolagus cuniculus
VITSSATTYYASWAKG

SEQ ID NO: 233 - protein, 17 aa, Oryctolagus cuniculus
CIYGDSSDTLYANWAKG

SEQ ID NO: 234 - protein, 18 aa, Oryctolagus cuniculus
CIFIGYGDVTWYASWAKG

SEQ ID NO: 235 - protein, 16 aa, Oryctolagus cuniculus
TIYIGGTTAYASWPKG

SEQ ID NO: 236 - protein, 18 aa, Oryctolagus cuniculus
CIYTGSSGITYYASWVNG

SEQ ID NO: 237 - protein, 16 aa, Oryctolagus cuniculus
VISSDGFFYDASWAKG

SEQ ID NO: 238 - protein, 16 aa, Oryctolagus cuniculus
IISGSASTYYATWAKG

SEQ ID NO: 239 - protein, 18 aa, Oryctolagus cuniculus
CIYAGSSGNTYYANWAKG

SEQ ID NO: 240 - protein, 16 aa, Oryctolagus cuniculus
SVISGGSTYYATWARG

SEQ ID NO: 241 - protein, 16 aa, Oryctolagus cuniculus
IIDSGGSAYYATWARG

SEQ ID NO: 242 - protein, 17 aa, Oryctolagus cuniculus
DIYAGSGSTWYASWAKG

SEQ ID NO: 243 - protein, 18 aa, Oryctolagus cuniculus
CIYANSVGSTYYASWVNG

SEQ ID NO: 244 - protein, 17 aa, Oryctolagus cuniculus
DIYPGSDSTWYASWAKG

SEQ ID NO: 245 - protein, 18 aa, Oryctolagus cuniculus
CIYAGSASNTYYATWVNG

SEQ ID NO: 246 - protein, 18 aa, Oryctolagus cuniculus
CIYAGSSGVTYYASWAKG

SEQ ID NO: 247 - protein, 17 aa, Oryctolagus cuniculus
YIYAGRDFTYYANWAEG

SEQ ID NO: 248 - protein, 16 aa, Oryctolagus cuniculus
YIGAGDITYCASWAKG

SEQ ID NO: 249 - protein, 18 aa, Oryctolagus cuniculus
CIYAGSSVSIYYATWAKG

SEQ ID NO: 250 - protein, 18 aa, Oryctolagus cuniculus
CIYAGSSGSTYYASWAKG

SEQ ID NO: 251 - protein, 18 aa, Oryctolagus cuniculus
CIYTGGSGVTYYASWAKG

SEQ ID NO: 252 - protein, 18 aa, Oryctolagus cuniculus
CIYAGSSGITYYASWVSG

SEQ ID NO: 253 - protein, 18 aa, Oryctolagus cuniculus
CIYAGSSDATYYANWVNG

SEQ ID NO: 254 - protein, 16 aa, Oryctolagus cuniculus
SVISGGSTYYATWAKG

SEQ ID NO: 255 - protein, 16 aa, Oryctolagus cuniculus
IIVSSGETFYASWARG

SEQ ID NO: 256 - protein, 17 aa, Oryctolagus cuniculus
YIYAAGPITYYATWAKG

SEQ ID NO: 257 - protein, 17 aa, Oryctolagus cuniculus
CIYTNSGNTWSASWAKG

SEQ ID NO: 258 - protein, 17 aa, Oryctolagus cuniculus
DIYIASDGTWYANWAKG

SEQ ID NO: 259 - protein, 18 aa, Oryctolagus cuniculus
CIYADSSSITYYASWVNG

SEQ ID NO: 260 - protein, 16 aa, Oryctolagus cuniculus
GIATDGNTYYANWAKG

SEQ ID NO: 261 - protein, 17 aa, Oryctolagus cuniculus
YIYAAGPITYYATWAKG

SEQ ID NO: 262 - protein, 16 aa, Oryctolagus cuniculus
IINSYGSIYYASWAKG

SEQ ID NO: 263 - protein, 18 aa, Oryctolagus cuniculus
CIYVGSSGSTYYASWVNG

SEQ ID NO: 264 - protein, 18 aa, Oryctolagus cuniculus
CIYVGSSGSTYYASWVSG

SEQ ID NO: 265 - protein, 17 aa, Oryctolagus cuniculus
YIYAAGPITYYATWAKG

SEQ ID NO: 266 - protein, 17 aa, Oryctolagus cuniculus
CIGTGDGLTYYASWAKG

SEQ ID NO: 267 - protein, 17 aa, Oryctolagus cuniculus
IIYAGSGGTYYASWVKG

SEQ ID NO: 268 - protein, 18 aa, Oryctolagus cuniculus
CIYTGSSGNTYYASWVNG

SEQ ID NO: 269 - protein, 17 aa, Oryctolagus cuniculus
CIYTSTGNTWYASWAKG

SEQ ID NO: 270 - protein, 17 aa, Oryctolagus cuniculus
DIYTGSGSTWYASWAKG

SEQ ID NO: 271 - protein, 16 aa, Oryctolagus cuniculus
IISNSGTTYYASWAKG

SEQ ID NO: 272 - protein, 18 aa, Oryctolagus cuniculus
CIYAGSSGSTYYANWVNG

SEQ ID NO: 273 - protein, 16 aa, Oryctolagus cuniculus
YMHVGGFPVYASWAKG

SEQ ID NO: 274 - protein, 18 aa, Oryctolagus cuniculus
CIYAGSSDSTYYASWVNG

SEQ ID NO: 275 - protein, 18 aa, Oryctolagus cuniculus
CIYAGSSGSTYYASWAKG

SEQ ID NO: 276 - protein, 17 aa, Oryctolagus cuniculus
CIYIGGGGRYYASWAKG

SEQ ID NO: 277 - protein, 17 aa, Oryctolagus cuniculus
CIGMGSGKTYYASWAKG

SEQ ID NO: 278 - protein, 16 aa, Oryctolagus cuniculus
SISSGGSTYYATWAKG

SEQ ID NO: 279 - protein, 16 aa, Oryctolagus cuniculus
IISSSGTTYYASWAKG

SEQ ID NO: 280 - protein, 16 aa, Oryctolagus cuniculus
TIDGGGSTYYASWAKG

SEQ ID NO: 281 - protein, 17 aa, Oryctolagus cuniculus
YINTGSGSTYYASWVNG

SEQ ID NO: 282 - protein, 18 aa, Oryctolagus cuniculus
CIYTGRSGGLYYANWAKG

SEQ ID NO: 283 - protein, 17 aa, Oryctolagus cuniculus
CIYTSTDTTYYPNWAKG

SEQ ID NO: 284 - protein, 18 aa, Oryctolagus cuniculus
CIVVGSGGNTYYAGWAKR

SEQ ID NO: 285 - protein, 16 aa, Oryctolagus cuniculus
VIDIENSVYYPTWAKG

SEQ ID NO: 286 - protein, 17 aa, Oryctolagus cuniculus
YINTGSGRTYYASWAKG

SEQ ID NO: 287 - protein, 16 aa, Oryctolagus cuniculus
FIGRGGATWYASWVKG

SEQ ID NO: 288 - protein, 16 aa, Oryctolagus cuniculus
FIGRGGSTWYASWVNG

SEQ ID NO: 289 - protein, 16 aa, Oryctolagus cuniculus
FIGRGGSTWYASWVNG

SEQ ID NO: 290 - protein, 16 aa, Oryctolagus cuniculus
IISSSGRTYYANWAKG

SEQ ID NO: 291 - protein, 16 aa, Oryctolagus cuniculus
MIYGSGYTYYASWAKG

SEQ ID NO: 292 - protein, 17 aa, Oryctolagus cuniculus
DIYAGSGSTWYASWVKG

SEQ ID NO: 293 - protein, 18 aa, Oryctolagus cuniculus
CIYAGSGDVTYYANWVNG

SEQ ID NO: 294 - protein, 16 aa, Oryctolagus cuniculus
TIYVSGRVYYATWAKG

SEQ ID NO: 295 - protein, 17 aa, Oryctolagus cuniculus
CIYTGSGGTYYASWEKG

SEQ ID NO: 296 - protein, 16 aa, Oryctolagus cuniculus
IILSSGSTYYATWAKG

SEQ ID NO: 297 - protein, 17 aa, Oryctolagus cuniculus
CIYTGSGSTYYANWAKG

SEQ ID NO: 298 - protein, 16 aa, Oryctolagus cuniculus
GIATDGNTYYANWAKG

SEQ ID NO: 299 - protein, 16 aa, Oryctolagus cuniculus
VIGSSGNLYYASWAKG

SEQ ID NO: 300 - protein, 16 aa, Oryctolagus cuniculus
ISVSNIRTWYATWAKG

SEQ ID NO: 301 - protein, 17 aa, Oryctolagus cuniculus
DIYAGSVNTWYATWAKG

SEQ ID NO: 302 - protein, 16 aa, Oryctolagus cuniculus
YINSLGGSYYASWAKG

SEQ ID NO: 303 - protein, 16 aa, Oryctolagus cuniculus
FIEPGGRAYCASWAKG

SEQ ID NO: 304 - protein, 18 aa, Oryctolagus cuniculus
CIYTGNSDFTYYANWAKG

SEQ ID NO: 305 - protein, 18 aa, Oryctolagus cuniculus
CINTGSGGSTYYANWVKG

SEQ ID NO: 306 - protein, 18 aa, Oryctolagus cuniculus
CIYAGSSDSTYYASWVNG

SEQ ID NO: 307 - protein, 17 aa, Oryctolagus cuniculus
CIYAGSSVTYYARWAKG

SEQ ID NO: 308 - protein, 16 aa, Oryctolagus cuniculus
FIGRGGSTWYASWVNG

SEQ ID NO: 309 - protein, 15 aa, Oryctolagus cuniculus
GGPGYSTNTHYAFDP

SEQ ID NO: 310 - protein, 9 aa, Oryctolagus cuniculus
YPGGSYYNL

SEQ ID NO: 311 - protein, 11 aa, Oryctolagus cuniculus
ALGSSGYRVNL

SEQ ID NO: 312 - protein, 12 aa, Oryctolagus cuniculus
LQGANYYNSLAL

SEQ ID NO: 313 - protein, 9 aa, Oryctolagus cuniculus
DGPSTLFNF

SEQ ID NO: 314 - protein, 11 aa, Oryctolagus cuniculus
DRGTSTGSLDL

SEQ ID NO: 315 - protein, 17 aa, Oryctolagus cuniculus
THYAAVAGYGYASRLDL

SEQ ID NO: 316 - protein, 11 aa, Oryctolagus cuniculus
VDASSSGSWDL

SEQ ID NO: 317 - protein, 14 aa, Oryctolagus cuniculus
GCPGYNGDKYTFDL

SEQ ID NO: 318 - protein, 15 aa, Oryctolagus cuniculus
GGPAYSTNTHYTLDL

SEQ ID NO: 319 - protein, 13 aa, Oryctolagus cuniculus
EIDAGYVGYGFNL

SEQ ID NO: 320 - protein, 12 aa, Oryctolagus cuniculus
VDPGYSFDAFDP

SEQ ID NO: 321 - protein, 13 aa, Oryctolagus cuniculus
EIDAGYVGYGFDL

SEQ ID NO: 322 - protein, 15 aa, Oryctolagus cuniculus
DRGYDDYGDITRLDL

SEQ ID NO: 323 - protein, 9 aa, Oryctolagus cuniculus
ETDGNYFNL

SEQ ID NO: 324 - protein, 11 aa, Oryctolagus cuniculus
DGGSPNWTLDL

SEQ ID NO: 325 - protein, 8 aa, Oryctolagus cuniculus
WGPGALDL

SEQ ID NO: 326 - protein, 11 aa, Oryctolagus cuniculus
STGSVGRGFNL

SEQ ID NO: 327 - protein, 11 aa, Oryctolagus cuniculus
VDGSSSGSWDL

SEQ ID NO: 328 - protein, 10 aa, Oryctolagus cuniculus
DLVVVTSFNL

SEQ ID NO: 329 - protein, 11 aa, Oryctolagus cuniculus
DIYASTSGYDL

SEQ ID NO: 330 - protein, 9 aa, Oryctolagus cuniculus
GVGFGYFNL

SEQ ID NO: 331 - protein, 14 aa, Oryctolagus cuniculus
GCPGYNGDKYALDL

SEQ ID NO: 332 - protein, 15 aa, Oryctolagus cuniculus
GGPGYSFDTEYAFDP

SEQ ID NO: 333 - protein, 15 aa, Oryctolagus cuniculus
DGSGSGTYGYNGMDL

SEQ ID NO: 334 - protein, 10 aa, Oryctolagus cuniculus
DLNYPDTSNL

SEQ ID NO: 335 - protein, 13 aa, Oryctolagus cuniculus
EIDAGYVGYGFNL

SEQ ID NO: 336 - protein, 11 aa, Oryctolagus cuniculus
DYGGSGYNFNL

SEQ ID NO: 337 - protein, 15 aa, Oryctolagus cuniculus
GGPAYSRGTHYAMDL

SEQ ID NO: 338 - protein, 15 aa, Oryctolagus cuniculus
DGSGSGTYGYNGMDL

SEQ ID NO: 339 - protein, 12 aa, Oryctolagus cuniculus
SAYSNNGDRLHL

SEQ ID NO: 340 - protein, 13 aa, Oryctolagus cuniculus
DGATSTSGHLFEL

SEQ ID NO: 341 - protein, 11 aa, Oryctolagus cuniculus
DIYGSTNGYDL

SEQ ID NO: 342 - protein, 15 aa, Oryctolagus cuniculus
DGSGSGTYGYNGMDL

SEQ ID NO: 343 - protein, 12 aa, Oryctolagus cuniculus
DRYATVSGILNL

SEQ ID NO: 344 - protein, 11 aa, Oryctolagus cuniculus
AVPDDSAGKKL

SEQ ID NO: 345 - protein, 10 aa, Oryctolagus cuniculus
DANSHYMMNL

SEQ ID NO: 346 - protein, 10 aa, Oryctolagus cuniculus
DLLVVTSFNL

SEQ ID NO: 347 - protein, 13 aa, Oryctolagus cuniculus
EIDAGYVGYGFNL

SEQ ID NO: 348 - protein, 12 aa, Oryctolagus cuniculus
DRYANTHGIFSL

SEQ ID NO: 349 - protein, 12 aa, Oryctolagus cuniculus
SIVDFSSGWGDL

SEQ ID NO: 350 - protein, 11 aa, Oryctolagus cuniculus
DFGPPNWTLDL

SEQ ID NO: 351 - protein, 11 aa, Oryctolagus cuniculus
PGYGGYGYYGL

SEQ ID NO: 352 - protein, 9 aa, Oryctolagus cuniculus
HAAWFELDL

SEQ ID NO: 353 - protein, 13 aa, Oryctolagus cuniculus
DPVTSGSDYVYDL

SEQ ID NO: 354 - protein, 11 aa, Oryctolagus cuniculus
KDGSGNEHYNL

SEQ ID NO: 355 - protein, 15 aa, Oryctolagus cuniculus
SDGYTNGDYDTYFNL

SEQ ID NO: 356 - protein, 15 aa, Oryctolagus cuniculus
ADGYTEGDYATYFNL

SEQ ID NO: 357 - protein, 13 aa, Oryctolagus cuniculus
NYYAGLSDVFFGW

SEQ ID NO: 358 - protein, 6 aa, Oryctolagus cuniculus
GLPSDL

SEQ ID NO: 359 - protein, 10 aa, Oryctolagus cuniculus
YIGAWGPWSL

SEQ ID NO: 360 - protein, 10 aa, Oryctolagus cuniculus
DLLVVTSFNL

SEQ ID NO: 361 - protein, 14 aa, Oryctolagus cuniculus
GSYDDYGDYWYFTL

SEQ ID NO: 362 - protein, 9 aa, Oryctolagus cuniculus
GDYIMTLDL

SEQ ID NO: 363 - protein, 6 aa, Oryctolagus cuniculus
GLPSDL

SEQ ID NO: 364 - protein, 12 aa, Oryctolagus cuniculus
DGDSSDYYAFNL

SEQ ID NO: 365 - protein, 12 aa, Oryctolagus cuniculus
DGDYSDYYTFDL

SEQ ID NO: 366 - protein, 12 aa, Oryctolagus cuniculus
DGDFSDYYTFNL

SEQ ID NO: 367 - protein, 14 aa, Oryctolagus cuniculus
LITVDYYIYDYFNL

SEQ ID NO: 368 - protein, 7 aa, Oryctolagus cuniculus
DPQYFIL

SEQ ID NO: 369 - protein, 13 aa, Oryctolagus cuniculus
EIDAGYVGYGFNL

SEQ ID NO: 370 - protein, 9 aa, Oryctolagus cuniculus
GVGFGYFNL

SEQ ID NO: 371 - protein, 7 aa, Oryctolagus cuniculus
GSIDYDP

SEQ ID NO: 372 - protein, 8 aa, Oryctolagus cuniculus
DPGYSSWL

SEQ ID NO: 373 - protein, 15 aa, Oryctolagus cuniculus
GGPGYSIDTKYAFDP

SEQ ID NO: 374 - protein, 10 aa, Oryctolagus cuniculus
NSNDWMYFNL

SEQ ID NO: 375 - protein, 15 aa, Oryctolagus cuniculus
GGPAYSRGTHYAMDL

SEQ ID NO: 376 - protein, 12 aa, Oryctolagus cuniculus
YTIDSGIYTYDL

SEQ ID NO: 377 - protein, 12 aa, Oryctolagus cuniculus
HVSRSGNYGLDL

SEQ ID NO: 378 - protein, 13 aa, Oryctolagus cuniculus
EIDAGYVGYGFNL

SEQ ID NO: 379 - protein, 7 aa, Oryctolagus cuniculus
DFAGSDL

SEQ ID NO: 380 - protein, 14 aa, Oryctolagus cuniculus
SYVFYSTYPYASDL

SEQ ID NO: 381 - protein, 10 aa, Oryctolagus cuniculus
FRDDYASLKL

SEQ ID NO: 382 - protein, 8 aa, Oryctolagus cuniculus
GDDSYYEL

SEQ ID NO: 383 - protein, 12 aa, Oryctolagus cuniculus
AIADFSSGWGDL

SEQ ID NO: 384 - skipped

SEQ ID NO: 385 - protein, 12 aa, Oryctolagus cuniculus
DGDYSDYYTFDL

SEQ ID NO: 386 - protein, 11 aa, Oryctolagus cuniculus
QASQSIYIYLS

SEQ ID NO: 387 - protein, 11 aa, Oryctolagus cuniculus
QASQTISINLA

SEQ ID NO: 388 - protein, 11 aa, Oryctolagus cuniculus
QASENIYSSLA

SEQ ID NO: 389 - protein, 11 aa, Oryctolagus cuniculus
QASQSIYSFLS

SEQ ID NO: 390 - protein, 11 aa, Oryctolagus cuniculus
QASEDIYSNLA

SEQ ID NO: 391 - protein, 11 aa, Oryctolagus cuniculus
QASENIGNGLA

SEQ ID NO: 392 - protein, 11 aa, Oryctolagus cuniculus
LASEDIYSGIS

SEQ ID NO: 393 - protein, 11 aa, Oryctolagus cuniculus
QASESIYSNLA

SEQ ID NO: 394 - protein, 11 aa, Oryctolagus cuniculus
QASQSIYNYLS

SEQ ID NO: 395 - protein, 11 aa, Oryctolagus cuniculus
QASQSIYNYLS

SEQ ID NO: 396 - protein, 12 aa, Oryctolagus cuniculus
QSSQSVYSDYLA

SEQ ID NO: 397 - protein, 11 aa, Oryctolagus cuniculus
QASEDIYSNLA

SEQ ID NO: 398 - protein, 12 aa, Oryctolagus cuniculus
QSSQSVYSDYLA

SEQ ID NO: 399 - protein, 11 aa, Oryctolagus cuniculus
QASEDIYSNLA

SEQ ID NO: 400 - protein, 11 aa, Oryctolagus cuniculus
QASEDIYSNLA

SEQ ID NO: 401 - protein, 11 aa, Oryctolagus cuniculus
QASESISDYLS

SEQ ID NO: 402 - protein, 13 aa, Oryctolagus cuniculus
QSSQSVYNNNYLS

SEQ ID NO: 403 - protein, 13 aa, Oryctolagus cuniculus
QASQSVYNSNHLS

SEQ ID NO: 404 - protein, 11 aa, Oryctolagus cuniculus
QASQNIYSNLA

SEQ ID NO: 405 - protein, 11 aa, Oryctolagus cuniculus
QASEDIYSNLA

SEQ ID NO: 406 - protein, 11 aa, Oryctolagus cuniculus
QASEDIYSNLA

SEQ ID NO: 407 - protein, 11 aa, Oryctolagus cuniculus
QASEDIYSNLA

SEQ ID NO: 408 - protein, 11 aa, Oryctolagus cuniculus
QASQSIHNYLS

SEQ ID NO: 409 - protein, 11 aa, Oryctolagus cuniculus
QASQSIGSWLS

SEQ ID NO: 410 - protein, 11 aa, Oryctolagus cuniculus
QASQSISNWLA

SEQ ID NO: 411 - protein, 11 aa, Oryctolagus cuniculus
QASQSIGYYLA

SEQ ID NO: 412 - protein, 12 aa, Oryctolagus cuniculus
QSSQSVYSDYLG

SEQ ID NO: 413 - protein, 11 aa, Oryctolagus cuniculus
QASEDIYSNLA

SEQ ID NO: 414 - protein, 11 aa, Oryctolagus cuniculus
QASQSIYIYLA

SEQ ID NO: 415 - protein, 11 aa, Oryctolagus cuniculus
QASQSISNWLA

SEQ ID NO: 416 - protein, 11 aa, Oryctolagus cuniculus
QASEDIYSNLA

SEQ ID NO: 417 - protein, 11 aa, Oryctolagus cuniculus
QASEDIYSNLA

SEQ ID NO: 418 - protein, 11 aa, Oryctolagus cuniculus
QASEDIYSNLA

SEQ ID NO: 419 - protein, 11 aa, Oryctolagus cuniculus
QASQSISNWLA

SEQ ID NO: 420 - protein, 11 aa, Oryctolagus cuniculus
QASQNIYSNCA

SEQ ID NO: 421 - protein, 11 aa, Oryctolagus cuniculus
QASENIYSSLA

SEQ ID NO: 422 - protein, 11 aa, Oryctolagus cuniculus
QASEDIYSNLA

SEQ ID NO: 423 - protein, 11 aa, Oryctolagus cuniculus
QASEDIYSNLA

SEQ ID NO: 424 - protein, 12 aa, Oryctolagus cuniculus
QSSQSVYSDYLV

SEQ ID NO: 425 - protein, 11 aa, Oryctolagus cuniculus
QASESIYSDLA

SEQ ID NO: 426 - protein, 11 aa, Oryctolagus cuniculus
QASEDIYSNLA

SEQ ID NO: 427 - protein, 11 aa, Oryctolagus cuniculus
QASQSISSYCS

SEQ ID NO: 428 - protein, 11 aa, Oryctolagus cuniculus
QASEDIYSNLA

SEQ ID NO: 429 - protein, 12 aa, Oryctolagus cuniculus
QASQNIASAYLS

SEQ ID NO: 430 - protein, 11 aa, Oryctolagus cuniculus
QASESISTWLA

SEQ ID NO: 431 - protein, 11 aa, Oryctolagus cuniculus
QASQSISSYLA

SEQ ID NO: 432 - protein, 11 aa, Oryctolagus cuniculus
QASQSISSWLS

SEQ ID NO: 433 - protein, 11 aa, Oryctolagus cuniculus
QASQSITTWLA

SEQ ID NO: 434 - protein, 11 aa, Oryctolagus cuniculus
QASETIYSGLA

SEQ ID NO: 435 - protein, 11 aa, Oryctolagus cuniculus
QASQSINSRLA

SEQ ID NO: 436 - protein, 12 aa, Oryctolagus cuniculus
QSSPSVYNNYLS

SEQ ID NO: 437 - protein, 11 aa, Oryctolagus cuniculus
QASEDIYSNLA

SEQ ID NO: 438 - protein, 11 aa, Oryctolagus cuniculus
QASENIYNFLA

SEQ ID NO: 439 - protein, 13 aa, Oryctolagus cuniculus
QASQSVYKNNRLA

SEQ ID NO: 440 - protein, 11 aa, Oryctolagus cuniculus
QASQSINSRLA

SEQ ID NO: 441 - protein, 11 aa, Oryctolagus cuniculus
QASQNIGSNLA

SEQ ID NO: 442 - protein, 11 aa, Oryctolagus cuniculus
QASQSIGSNLA

SEQ ID NO: 443 - protein, 11 aa, Oryctolagus cuniculus
QASQNIGSNLA

SEQ ID NO: 444 - protein, 11 aa, Oryctolagus cuniculus
QASQSISSYLS

SEQ ID NO: 445 - protein, 13 aa, Oryctolagus cuniculus
QSSQSVDGNNLLS

SEQ ID NO: 446 - protein, 12 aa, Oryctolagus cuniculus
QSSQSVYSDYLA

SEQ ID NO: 447 - protein, 11 aa, Oryctolagus cuniculus
QASEDIYSNLA

SEQ ID NO: 448 - protein, 11 aa, Oryctolagus cuniculus
QASQSISSWLS

SEQ ID NO: 449 - protein, 11 aa, Oryctolagus cuniculus
QASESISNYLS

SEQ ID NO: 450 - protein, 11 aa, Oryctolagus cuniculus
QASQNIYIYLS

SEQ ID NO: 451 - protein, 12 aa, Oryctolagus cuniculus
QASESISANYWS

SEQ ID NO: 452 - protein, 11 aa, Oryctolagus cuniculus
QASQSIYIYLA

SEQ ID NO: 453 - protein, 13 aa, Oryctolagus cuniculus
QSSQSVYGNNELS

SEQ ID NO: 454 - protein, 11 aa, Oryctolagus cuniculus
QASEDISSYLA

SEQ ID NO: 455 - protein, 12 aa, Oryctolagus cuniculus
QSSQSVYSDYLA

SEQ ID NO: 456 - protein, 11 aa, Oryctolagus cuniculus
QASQSIDYYLA

SEQ ID NO: 457 - protein, 13 aa, Oryctolagus cuniculus
QSSQSVNGNNYLA

SEQ ID NO: 458 - protein, 11 aa, Oryctolagus cuniculus
QASQSISSYLS

SEQ ID NO: 459 - protein, 11 aa, Oryctolagus cuniculus
QASQNIYSGLA

SEQ ID NO: 460 - protein, 11 aa, Oryctolagus cuniculus
QASEDIYSNLA

SEQ ID NO: 461 - protein, 13 aa, Oryctolagus cuniculus
QASQSVYKNNYLS

SEQ ID NO: 462 - protein, 11 aa, Oryctolagus cuniculus
QASQNIGSNLA

SEQ ID NO: 463 - protein, 7 aa, Oryctolagus cuniculus
DASKLAS

SEQ ID NO: 464 - protein, 7 aa, Oryctolagus cuniculus
YASTLAS

SEQ ID NO: 465 - protein, 7 aa, Oryctolagus cuniculus
DASDLAS

SEQ ID NO: 466 - protein, 7 aa, Oryctolagus cuniculus
AASDLES

SEQ ID NO: 467 - protein, 7 aa, Oryctolagus cuniculus
RASTLAS

SEQ ID NO: 468 - protein, 7 aa, Oryctolagus cuniculus
GASTLAS

SEQ ID NO: 469 - protein, 7 aa, Oryctolagus cuniculus
AASNLES

SEQ ID NO: 470 - protein, 7 aa, Oryctolagus cuniculus
AASLLAS

SEQ ID NO: 471 - protein, 7 aa, Oryctolagus cuniculus
RASTLAS

SEQ ID NO: 472 - protein, 7 aa, Oryctolagus cuniculus
DASELAS

SEQ ID NO: 473 - protein, 7 aa, Oryctolagus cuniculus
QASKLAT

SEQ ID NO: 474 - protein, 7 aa, Oryctolagus cuniculus
RASTLAS

SEQ ID NO: 475 - protein, 7 aa, Oryctolagus cuniculus
KASKLAS

SEQ ID NO: 476 - protein, 7 aa, Oryctolagus cuniculus
RASTLAS

SEQ ID NO: 477 - protein, 7 aa, Oryctolagus cuniculus
RASTLAS

SEQ ID NO: 478 - protein, 7 aa, Oryctolagus cuniculus
RASTLES

SEQ ID NO: 479 - protein, 7 aa, Oryctolagus cuniculus
KASTLAS

SEQ ID NO: 480 - protein, 7 aa, Oryctolagus cuniculus
SASTLAS

SEQ ID NO: 481 - protein, 7 aa, Oryctolagus cuniculus
AASLLAS

SEQ ID NO: 482 - protein, 7 aa, Oryctolagus cuniculus
SASTLAS

SEQ ID NO: 483 - protein, 7 aa, Oryctolagus cuniculus
QASTLAS

SEQ ID NO: 484 - protein, 7 aa, Oryctolagus cuniculus
DASTLAS

SEQ ID NO: 485 - protein, 7 aa, Oryctolagus cuniculus
RASTLAS

SEQ ID NO: 486 - protein, 7 aa, Oryctolagus cuniculus
RASTLAS

SEQ ID NO: 487 - protein, 7 aa, Oryctolagus cuniculus
RASTLAS

SEQ ID NO: 488 - protein, 7 aa, Oryctolagus cuniculus
RASTLAS

SEQ ID NO: 489 - protein, 7 aa, Oryctolagus cuniculus
WASKLET

SEQ ID NO: 490 - protein, 7 aa, Oryctolagus cuniculus
YASTLAS

SEQ ID NO: 491 - protein, 7 aa, Oryctolagus cuniculus
DASKLAS

SEQ ID NO: 492 - protein, 7 aa, Oryctolagus cuniculus
RASTLAS

SEQ ID NO: 493 - protein, 7 aa, Oryctolagus cuniculus
YVSTLES

SEQ ID NO: 494 - protein, 7 aa, Oryctolagus cuniculus
RASTLAS

SEQ ID NO: 495 - protein, 7 aa, Oryctolagus cuniculus
QASKLAS

SEQ ID NO: 496 - protein, 7 aa, Oryctolagus cuniculus
RASTLAS

SEQ ID NO: 497 - protein, 7 aa, Oryctolagus cuniculus
YVSTLES

SEQ ID NO: 498 - protein, 7 aa, Oryctolagus cuniculus
DASTLAS

SEQ ID NO: 499 - protein, 7 aa, Oryctolagus cuniculus
DASTLAS

SEQ ID NO: 500 - protein, 7 aa, Oryctolagus cuniculus
GASTLAS

SEQ ID NO: 501 - protein, 7 aa, Oryctolagus cuniculus
QASKLAS

SEQ ID NO: 502 - protein, 7 aa, Oryctolagus cuniculus
FVATLES

SEQ ID NO: 503 - protein, 7 aa, Oryctolagus cuniculus
GVSTLAS

SEQ ID NO: 504 - protein, 7 aa, Oryctolagus cuniculus
RASTLES

SEQ ID NO: 505 - protein, 7 aa, Oryctolagus cuniculus
DASTLAS

SEQ ID NO: 506 - protein, 7 aa, Oryctolagus cuniculus
AASTLTD

SEQ ID NO: 507 - protein, 7 aa, Oryctolagus cuniculus
RASTLAS

SEQ ID NO: 508 - protein, 7 aa, Oryctolagus cuniculus
KASTLAS

SEQ ID NO: 509 - protein, 7 aa, Oryctolagus cuniculus
QASALAS

SEQ ID NO: 510 - protein, 7 aa, Oryctolagus cuniculus
QASALAS

SEQ ID NO: 511 - protein, 7 aa, Oryctolagus cuniculus
YTSSLAS

SEQ ID NO: 512 - protein, 7 aa, Oryctolagus cuniculus
SASTLAS

SEQ ID NO: 513 - protein, 7 aa, Oryctolagus cuniculus
GASSLAS

SEQ ID NO: 514 - protein, 7 aa, Oryctolagus cuniculus
GASTLAS

SEQ ID NO: 515 - protein, 7 aa, Oryctolagus cuniculus
VASKLAS

SEQ ID NO: 516 - protein, 7 aa, Oryctolagus cuniculus
LASTLAS

SEQ ID NO: 517 - protein, 7 aa, Oryctolagus cuniculus
DASTLAS

SEQ ID NO: 518 - protein, 7 aa, Oryctolagus cuniculus
GASTLAS

SEQ ID NO: 519 - protein, 7 aa, Oryctolagus cuniculus
GASNLES

SEQ ID NO: 520 - protein, 7 aa, Oryctolagus cuniculus
GASTLAS

SEQ ID NO: 521 - protein, 7 aa, Oryctolagus cuniculus
GASTLAS

SEQ ID NO: 522 - protein, 7 aa, Oryctolagus cuniculus
DASNLAS

SEQ ID NO: 523 - protein, 7 aa, Oryctolagus cuniculus
WASKLAS

SEQ ID NO: 524 - protein, 7 aa, Oryctolagus cuniculus
DASTLAS

SEQ ID NO: 525 - protein, 7 aa, Oryctolagus cuniculus
RASTLAS

SEQ ID NO: 526 - protein, 7 aa, Oryctolagus cuniculus
LASTLAS

SEQ ID NO: 527 - protein, 7 aa, Oryctolagus cuniculus
DASTLAS

SEQ ID NO: 528 - protein, 7 aa, Oryctolagus cuniculus
GASTLAS

SEQ ID NO: 529 - protein, 7 aa, Oryctolagus cuniculus
DASKLAS

SEQ ID NO: 530 - protein, 7 aa, Oryctolagus cuniculus
GASILAS

SEQ ID NO: 531 - protein, 7 aa, Oryctolagus cuniculus
DASDLAS

SEQ ID NO: 532 - protein, 7 aa, Oryctolagus cuniculus
QASKLAS

SEQ ID NO: 533 - protein, 7 aa, Oryctolagus cuniculus
RASTLAS

SEQ ID NO: 534 - protein, 7 aa, Oryctolagus cuniculus
LASSLAS

SEQ ID NO: 535 - protein, 7 aa, Oryctolagus cuniculus
GASNLAS

SEQ ID NO: 536 - protein, 7 aa, Oryctolagus cuniculus
YASTLAS

SEQ ID NO: 537 - protein, 7 aa, Oryctolagus cuniculus
GASTLAS

SEQ ID NO: 538 - protein, 7 aa, Oryctolagus cuniculus
SASTLDS

SEQ ID NO: 539 - protein, 7 aa, Oryctolagus cuniculus
GASTLAS

SEQ ID NO: 540 - protein, 12 aa, Oryctolagus cuniculus
QQGATTYNVDNV

SEQ ID NO: 541 - protein, 12 aa, Oryctolagus cuniculus
QQGYTEDNIDNT

SEQ ID NO: 542 - protein, 13 aa, Oryctolagus cuniculus
QQGYYSGGTDNDV

SEQ ID NO: 543 - protein, 10 aa, Oryctolagus cuniculus
QCNYIIDYGA

SEQ ID NO: 544 - protein, 12 aa, Oryctolagus cuniculus
LGVYTYPSADNA

SEQ ID NO: 545 - protein, 13 aa, Oryctolagus cuniculus
QCTYWNPDYIGGA

SEQ ID NO: 546 - protein, 12 aa, Oryctolagus cuniculus
LGGYSYSNTGPT

SEQ ID NO: 547 - protein, 12 aa, Oryctolagus cuniculus
QSASYSTGPDWT

SEQ ID NO: 548 - protein, 12 aa, Oryctolagus cuniculus
QQGATSYDIENP

SEQ ID NO: 549 - protein, 12 aa, Oryctolagus cuniculus
QQGATTYNIENV

SEQ ID NO: 550 - protein, 12 aa, Oryctolagus cuniculus
QATYYGSGWYRA

SEQ ID NO: 551 - protein, 13 aa, Oryctolagus cuniculus
LGVRTYFNTLNNS

SEQ ID NO: 552 - protein, 12 aa, Oryctolagus cuniculus
QATYYSVGWYRA

SEQ ID NO: 553 - protein, 13 aa, Oryctolagus cuniculus
LGVYTYLSDLFFV

SEQ ID NO: 554 - protein, 12 aa, Oryctolagus cuniculus
LGVYTYSTDIHA

SEQ ID NO: 555 - protein, 11 aa, Oryctolagus cuniculus
QSNYYDSRGNA

SEQ ID NO: 556 - protein, 10 aa, Oryctolagus cuniculus
AGFYETTDVG

SEQ ID NO: 557 - protein, 13 aa, Oryctolagus cuniculus
QGEFSCVSADCIA

SEQ ID NO: 558 - protein, 12 aa, Oryctolagus cuniculus
QGAVYSGNTEWA

SEQ ID NO: 559 - protein, 15 aa, Oryctolagus cuniculus
LGVCTDISVDDVYNS

SEQ ID NO: 560 - protein, 16 aa, Oryctolagus cuniculus
LGVCTYIGADNTLYNT

SEQ ID NO: 561 - protein, 12 aa, Oryctolagus cuniculus
LGVYTHISADNA

SEQ ID NO: 562 - protein, 12 aa, Oryctolagus cuniculus
QQGATSYDIDNA

SEQ ID NO: 563 - protein, 12 aa, Oryctolagus cuniculus
QQGATTYDVDNV

SEQ ID NO: 564 - protein, 12 aa, Oryctolagus cuniculus
QQGASTTDVDNV

SEQ ID NO: 565 - protein, 10 aa, Oryctolagus cuniculus
QSYYNSDSDA

SEQ ID NO: 566 - protein, 12 aa, Oryctolagus cuniculus
QATYYGSGWYRA

SEQ ID NO: 567 - protein, 15 aa, Oryctolagus cuniculus
LGVCTYINANGWDNA

SEQ ID NO: 568 - protein, 12 aa, Oryctolagus cuniculus
QQGATIWNVDNP

SEQ ID NO: 569 - protein, 12 aa, Oryctolagus cuniculus
QQGASTTDVDNV

SEQ ID NO: 570 - protein, 12 aa, Oryctolagus cuniculus
QCTEGGSGSDYT

SEQ ID NO: 571 - protein, 16 aa, Oryctolagus cuniculus
LGIYTYISADGSLYNA

SEQ ID NO: 572 - protein, 16 aa, Oryctolagus cuniculus
LGVGTYISGDGSLDNA

SEQ ID NO: 573 - protein, 12 aa, Oryctolagus cuniculus
QQGASTTDVDNV

SEQ ID NO: 574 - protein, 13 aa, Oryctolagus cuniculus
QYTYDSSSSTSWA

SEQ ID NO: 575 - protein, 14 aa, Oryctolagus cuniculus
QSYYCSVSSSCGYG

SEQ ID NO: 576 - protein, 17 aa, Oryctolagus cuniculus
LGVYTYISADGTLVYNA

SEQ ID NO: 577 - protein, 15 aa, Oryctolagus cuniculus
LGVCTDISTDDLYNA

SEQ ID NO: 578 - protein, 12 aa, Oryctolagus cuniculus
QATYSSTGWYRA

SEQ ID NO: 579 - protein, 14 aa, Oryctolagus cuniculus
QCTYGGSGSGNGAA

SEQ ID NO: 580 - protein, 13 aa, Oryctolagus cuniculus
LGVYTYISDVYYT

SEQ ID NO: 581 - protein, 11 aa, Oryctolagus cuniculus
QSSYYDLLGNG

SEQ ID NO: 582 - protein, 15 aa, Oryctolagus cuniculus
LGVYTYISPDGTDNA

SEQ ID NO: 583 - protein, 12 aa, Oryctolagus cuniculus
AGYKSYTDDEFA

SEQ ID NO: 584 - protein, 12 aa, Oryctolagus cuniculus
QQGYTVNNIDNV

SEQ ID NO: 585 - protein, 12 aa, Oryctolagus cuniculus
QQGYASSGVDNV

SEQ ID NO: 586 - protein, 15 aa, Oryctolagus cuniculus
QCTYGIGSNSDYGVA

SEQ ID NO: 587 - protein, 15 aa, Oryctolagus cuniculus
QCTYGIGSGSSYGVA

SEQ ID NO: 588 - protein, 15 aa, Oryctolagus cuniculus
QTYYDSEGRSYGYNS

SEQ ID NO: 589 - protein, 14 aa, Oryctolagus cuniculus
LSHYLTSSSSYGDA

SEQ ID NO: 590 - protein, 12 aa, Oryctolagus cuniculus
QGGYNSYSDTFA

SEQ ID NO: 591 - protein, 15 aa, Oryctolagus cuniculus
LGVCTDISADDLYNT

SEQ ID NO: 592 - protein, 12 aa, Oryctolagus cuniculus
QQTYRYNDGDTA

SEQ ID NO: 593 - protein, 11 aa, Oryctolagus cuniculus
AGGYSTISENA

SEQ ID NO: 594 - protein, 14 aa, Oryctolagus cuniculus
LSHYLTSSSSYGNA

SEQ ID NO: 595 - protein, 12 aa, Oryctolagus cuniculus
QCSGYDITGVFP

SEQ ID NO: 596 - protein, 12 aa, Oryctolagus cuniculus
QCSGYDTTGVFP

SEQ ID NO: 597 - protein, 12 aa, Oryctolagus cuniculus
QCSGYDTTGVFP

SEQ ID NO: 598 - protein, 12 aa, Oryctolagus cuniculus
QQGYSYNNVDNT

SEQ ID NO: 599 - protein, 12 aa, Oryctolagus cuniculus
QGSYYSSSWYNV

SEQ ID NO: 600 - protein, 12 aa, Oryctolagus cuniculus
QATYNGRGWYRA

SEQ ID NO: 601 - protein, 12 aa, Oryctolagus cuniculus
LGVYTHISADNA

SEQ ID NO: 602 - protein, 13 aa, Oryctolagus cuniculus
QQGYITSSNIKNV

SEQ ID NO: 603 - protein, 12 aa, Oryctolagus cuniculus
QNWWVIEHNGAA

SEQ ID NO: 604 - protein, 12 aa, Oryctolagus cuniculus
QQGATTYDVDNV

SEQ ID NO: 605 - protein, 15 aa, Oryctolagus cuniculus
QSWYYSGSGSYHSWA

SEQ ID NO: 606 - protein, 12 aa, Oryctolagus cuniculus
QQGATIWNVDNP

SEQ ID NO: 607 - protein, 11 aa, Oryctolagus cuniculus
AGGYSSTSDNA

SEQ ID NO: 608 - protein, 13 aa, Oryctolagus cuniculus
QCADYATTYGLGA

SEQ ID NO: 609 - protein, 12 aa, Oryctolagus cuniculus
QATYSSSGWYRA

SEQ ID NO: 610 - protein, 12 aa, Oryctolagus cuniculus
QQGYSGNNVDNT

SEQ ID NO: 611 - protein, 12 aa, Oryctolagus cuniculus
AGAYSTSGEENA

SEQ ID NO: 612 - protein, 12 aa, Oryctolagus cuniculus
QCTYYDNNYGGA

SEQ ID NO: 613 - protein, 12 aa, Oryctolagus cuniculus
QTYYGVYVYGII

SEQ ID NO: 614 - protein, 13 aa, Oryctolagus cuniculus
LGVCTDISAVYNV

SEQ ID NO: 615 - protein, 13 aa, Oryctolagus cuniculus
QGNYDCSSADCIA

SEQ ID NO: 616 - protein, 12 aa, Oryctolagus cuniculus
QCSGYDTTGVFP